(19) United States
(12) Patent Application Publication
(10) Pub. No.: US 2007/0172847 A1
(43) Pub. Date: Jul. 26, 2007

(54) MOLECULAR SIGNALING PATHWAYS TRIGGERED BY RITUXIMAB: PROGNOSTIC, DIAGNOSTIC, AND THERAPEUTIC USES

(75) Inventors: Benjamin Bonavida, Los Angeles, CA (US); Ali R. Jazirehi, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/560,308

(22) Filed: Nov. 15, 2006

Related U.S. Application Data
(60) Provisional application No. 60/737,301, filed on Nov. 15, 2005.

(51) Int. Cl. C12Q 1/68; G01N 33/574

(52) U.S. Cl. 435/6; 435/7.23

(57) ABSTRACT

The present invention provides markers associated with activated molecular signaling pathways (example: p38 MAKP, NF-κB, ERK1/2, YY-1 and AKT) inhibited by rituximab in cancer cells as well as pathways activated by rituximab (such as death receptors, RKIP, PTEN) all of which are associated with the regulation of chemo and immunoresistance. The present invention provides methods of prognosis and providing a prognosis for cancer such as lymphoma, leukemia, and autoimmune disease, as well as, methods of drug discovery. These markers are also therapeutic targets for treatment of cancer resistant to conventional and experimental cancer therapeutics. Inhibition or activation of expression and/or activity of targeted gene products sensitizes resistant tumor cells to subtoxic doses of cytotoxic treatment including chemotherapy, radiation therapy, or immunotherapy and gene therapy, and the cytotoxic molecules.

### STATEMENT AS TO RIGHTS TO INVENTIONS MADE UNDER FEDERALLY SPONSORED RESEARCH OR DEVELOPMENT

This invention was made with Government support under Department of Defense/US Army Grant DAMD 17-02-1-0023. The Government has certain rights in this invention.

### CROSS-REFERENCES TO RELATED APPLICATIONS

Not applicable.

### BACKGROUND OF THE INVENTION

Cancer is the second leading cause of death behind heart disease. Cancer incidence and death figures account for about 10% of the U.S. population in certain areas of the United States (National Cancer Institute's Surveillance, Epidemiology, and End Results (SEER) database and Bureau of the Census statistics; see, Harrison's Principles of Internal Medicine, Kasper et al., 16^{th} ed., 2005, Chapter 66). The five leading causes of cancer deaths among men are lung cancer, prostate cancer, colon and rectum cancer, pancreatic cancer and leukemias. The five leading causes of cancer deaths among women are lung cancer, breast cancer, colon cancer, ovarian cancer and pancreatic cancer. When detected at locally advanced or metastatic stages, no consistently curative treatment regimen exists. Treatment for metastatic cancer includes hormonal ablation, radiation therapy, chemotherapy, hormonal therapy and combination therapies. Unfortunately, a resistance often develops to further hormonal manipulation or to treatment with conventional chemotherapy. Therefore, there is a need for alternative therapies, such as immunotherapy or reversal of resistance to chemotherapy, radiation therapy, and hormonal therapy. For instance, immunotherapy is predicated on the notion that all drug-resistant tumors should succumb to cytotoxic lymphocyte-mediated killing. Such tumors may also develop cross-resistance to apoptosis mediated by cytotoxic lymphocytes, resulting ultimately in tumor progression and metastasis of the resistant cells (Thompson, C., Science, 267:1456-62 (1995)). The mechanism responsible for the apoptotic-resistant phenotype, if identified, may be useful as a prognostic and/or diagnostic indicator and may serve as a target for immunotherapeutic intervention in the reversal of resistance to other cytotoxic therapies.

The phosphatidylinositol 3-Kinase (PI3-K) is formed by heterodimeric lipid kinases that catalyse the phosphorylation of inositol-containing lipids, known as phosphatidylinositol (PtdIns), allowing the conversion of phosphatidylinositol-3,4-biphosphate (PtdIns-P2) to phosphatidylinositol-3,4,5-triphosphate (PtdIns-P3). The latter is absent or undetectable in resting cells and although PI3-K activity in normal cells is tightly regulated, it is deregulated in a wide spectrum of tumors (Toker, A. et al., Cancer Res., 66:3963-6 (2006); Noske, A. et al., Cancer letter, 11: [Epub ahead of print] (2006); Guo, R. et al., J Steroid Biochem Mol Biol., 99:9-18 (2006); Koul, D. et al., Mol Cancer Ther., 5:637-44 (2006); Liu, X. et al., Mol Cancer Ther., 5:494-501(2006)). Akt is a serine/threonine protein kinase that mediates various downstream effects of PI3-K. It plays a central role in signaling by the PI3-K pathway by regulating many biological processes such as proliferation, apoptosis and cell growth; moreover, it was suggested to be involved in PI3-K-mediated tumorigenesis (Liu, X. et al., Mol Cancer Ther., 5:494-501(2006); Castilla, C. et al., Endocrinology [Epub ahead of print] (2006)). The Akt pathway is of particular interest because it regulates several critical cellular functions, including cell cycle progression, migration, invasion, and survival as well as angiogenesis. In addition, the activated PI3K-Akt pathway provides major survival signals to lymphoma cells and many other cancer cells (Toker, A. et al., Cancer Res., 66:3963-6 (2006); Goswami, A. et al., Cancer Res., 66:2889-92 (2006)). Akt controls a variety of mechanisms that inhibit apoptosis and prolong cell survival, exerting a positive effect on NF-κB functions (Osaki, M. et al., Apoptosis, 9:667-76 (2004); Ozes, O. et al., Nature, 401:82-5 (1999)).

The lymphatic cancers known as non-Hodgkin's lymphoma (NHL) are steadily increasing in prevalence worldwide. Although NHLs initially respond to a variety of therapeutic modalities, they exhibit an unremitting relapsing nature and are essentially considered incurable. This pattern of inevitable failure of standard therapies is due to the emergence of drug-resistant variants which highlights the urgent need for the design of new treatment regimens. Monoclonal antibodies (mAbs) targeted against specific surface markers that are less systematically toxic and less myelosuppressive, have provided an alternative therapeutic approach.

About 80-85% of NHLs are of B-cell origin and ˜95% of these express surface CD20 (1,2). One of the candidate antigens that has been targeted for immunotherapy is CD20, a 297-amino acid (32-37 kDa) unglycosylated phosphoprotein that spans the membrane four times (Ernst, J. et al., Biochemistry, 44:15150-8 (2005)). CD20 is a cell surface phosphoprotein that is expressed specifically within the B-cell lineage from pre-B cells to mature B cells. It is neither shed from the cell surface nor modulated or internalized on antibody binding (Ernst, J. et al., Biochemistry, 44:15150-8 (2005)). Rituximab (chimeric anti-human CD20 antibody) mediates its anti-tumor activity by multiple mechanisms that include complement-dependent cytotoxicity (CDC), antibody-dependent cellular cytotoxicity (ADCC) and induction of apoptosis following CD20 cross-linking (Shan, D. et al., Blood, 91:1644-52 (1998); Jazirehi, A. et al., Oncogene, 24:2121-43 (2005)). We have recently reported that Rituximab sensitizes drug-resistant B Non-Hodgkin's lymphoma (NHL) cell lines to the apoptotic effects of various chemotherapeutic drugs via the selective downregulation of Bcl-_{XL} expression. Downregulation of Bcl-_{XL} expression was the result of inhibition of both the Raf/MEK/ERK1/2 and NF-κB survival pathways (Jazirehi, A. et al., Cancer Res., 64:7117-26 (2004); Jazirehi, A. et al., Cancer Res., 65:264-76 (2005)).

While rituximab has been successfully used in the treatment of patients with non-Hodgkins lymphoma (NHL) its modes of action, however, have not yet been fully elucidated. It has been reported that the induction of antibody-dependent cell-mediated cytotoxicity (ADCC), complement dependent cytoxicity (CDC), and seldom induction of apoptosis may explain the efficacy of rituximab in vivo. Supporting data for these mechanisms have been reported both in vitro and in vivo and can be found in reviews (Smith, M., Oncogene, 22(47):7359-68 (2003); Jazirehi, A. et al., Oncogene, 24(13):2121-43 (2005)). The role of rituximab signaling in NHL cells and its induced modification of intracellular survival signaling pathways that regulate the proliferation states, the expression of surface receptors and antiapoptotic pathways has not been considered initially as potential mechanisms of rituximab mediated effects. Further, the chemo sensitizing effect of rituximab, initially reported by us (Demidem, A. et al., Cancer Biother Radiopharm., 12(3):177-86 (1997); Alas, S. et al., Cancer Res, 61:5137-44 (2001); Jazirehi, et al., 2003), and its underlying molecular mechanisms were not examined. Alterations of cell signaling upon administration of crosslinker (dimeric) ribuximab has been reported following crosslinking of this antibody with a secondary antibody (Deans, J. et al., J Immunol. 151(9):4494-504 (1993); Deans, J. et al., J Biol Chem. 270(38):22632-8 (1995)). However, intracellular events triggered by monomeric rituximab was not examined in these studies. The molecular signaling observed following cross-linking are very distinct from the one reported in this invention using monomeric rituximab.

Some NHL patients do not respond to rituximab treatment alone and it is not clear why such patients are unresponsive. It has been proposed that some of those patients exhibit polymorphism in their Fc receptors expressed on their tumor cells, making such cells resistant to ADCC (Cartron, G. et al., Blood, 99(3):754-8 (2002); Johnson, P. et al., Semin Oncol., 30(1 Suppl 2):3-8 (2003)). Thus, prior to the present invention, the state of art with respect to signaling with monomeric rituximab was not known. In this invention, several intracellular signaling pathways shown to be modified by rituximab are further shown to be important in the regulation of the tumor cell response to rituximab treatment alone or in combination with chemotherapeutic drugs. This invention identifies pathways that are modified by rituximab in which several gene products regulate the response to apoptotic stimuli (e.g., chemotherapy, immunotherapy) following treatment with rituximab. This invention also identifies gene products whose level of expression may dictate tumor cells response to conventional treatment. The invention therefore also identifies gene products that are targets for therapeutic intervention. Failure of chemotherapy to eliminate tumor cells has prompted the development of alternative therapies.

### BRIEF SUMMARY OF THE INVENTION

The present invention provides markers associated with molecular signaling pathways such as functional or activated AKT, NFκB, ERK 1/2, and p38MAPK that are triggered by rituximab in CD-20 expressing cancer cells, including polypeptide members of the pathways such as functional or activated Bcl-2/Bcl-_{XL}, AKT, PTEN, Fas, YY1, NFκB, NIK, IKK, IKB, and transcription factors AP-1 and STAT3. These markers are therefore useful as diagnostic and prognostic markers as well as for therapeutic intervention targets (e.g., in drug assays and patient treatment). The signaling pathways modified by rituximab are implicated in the sensitization of tumor cells to death receptor mediated apoptotic pathways (FasL, Trail, TNFα) cells and sensitize the cells to cytotoxic immunotherapy (FASL, TNF, TRAIL) which are potent in immunotherapeutic approaches to cancer treatment.

The chimeric mouse and human anti-CD-20 monoclonal antibody rituximab (RITUXAN, IDEC-C2B8) has been approved by the FDA for the treatment of B-Non Hodgkin's lymphoma (NHL). It has significant anti-tumor activity and, alone or in combination with chemotherapy, and has been successfully used in the treatment of patients with follicular or low grade NHL (Czucman, M. et al., Semin. Oncol, 29:36-40 (2002)) and aggressive diffuse large B cell lymphoma (DLBCL) in elderly patients (Coiffer, B., Semin Oncol, 30:21-7 (2003)). Rituximab treatment depletes CD20 positive normal and cancerous B cells in patients. The postulated mechanisms of rituximab-mediated effects include antibody dependent cellular cytoxicity (ADCC), complement dependent cytotoxicity (CDC), and induction of apoptosis (Maloney, D. et al., Semin. Oncol., 29:2-9 (2002); Smith, M., Oncogene, 22(47):7359-68 (2003); Jazirehi, A. et al., Oncogene. 24(13):2121-43 (2005)). However, these postulated mechanisms do not explain the failure of approximately 50% of NHL patients to respond to rituximab treatment alone and do not explain the enhanced response achieved with treatment combination of rituximab and chemotherapeutic drugs in patients with drug-resistant tumors.

This invention describes novel mechanisms of rituximab-mediated activity which explain the underlying basis of failure to respond to rituximab treatment alone and also explains the molecular mechanism of rituximab-mediated sensitization to chemotherapeutic drug-induced apoptosis in drug resistant B-NHL. This invention describes the molecular signaling pathways triggered by rituximab that result in the specific modifications of cell survival signaling pathways utilized by the tumor cells and which will result in the inhibition of cell proliferation and growth, inhibition of gene products associated with resistance to apoptosis and significant sensitization to a variety of chemotherapeutic drugs. This invention also identifies a number of intracellular gene products that are modified selectively by rituximab and which are therefore molecular targets for the same indications as rituximab. In addition, the signaling pathways modified by rituximab in rituximab sensitive NHL cell lines identify gene products whose over expression or otherwise modification or mutation are involved in the resistance to rituximab mediated affects. In addition, this invention can be utilized to evaluate patient's tumors for a response or lack of response to rituximab based on the profile of the signaling pathways modulated by rituximab and thus has significant diagnostic/prognostic clinical significance. While the above studies were performed in B-NHL cell lines, the findings are applicable for other applications by rituximab, currently under intensive investigations, in the treatment of other B cell tumors and B cell mediated diseases such as autoimmunity, rheumatoid arthritis, lupus, transplantation, etc.

Generally, the methods find particular use in diagnosing or providing a prognosis for cancer including prostate cancer, renal cancer, lung cancer, ovarian cancer, breast cancer, colon cancer, leukemias, B-cell lymphomas (e.g., non-Hodgkin's lymphomas, including Burkitt's, small cell, and diffuse large cell lymphomas), hepatocarcinoma or multiple myeloma. For example, these markers are useful for profiling a cancer patient to determine their sensitivity or resistance to rituxamib therapy, and for in vivo imaging. In addition, the methods find use in drug assays for cancer therapeutics, including the aforementioned cancers.

Accordingly, in one aspect the invention provides a method of diagnosing a cancer or providing a prognosis for a cancer for a patient that has altered expression of molecular signaling pathways triggered by rituximab by determining whether or not expression or amounts of a protein that is part of a molecular signaling pathway triggered by rituximab is altered in a tissue sample of the cancer from a patient, thereby diagnosing or providing the prognosis for the cancer. In some embodiments, the tissue sample is contacted with an antibody that specifically binds to protein that is part of a molecular signaling pathway triggered by rituximab; and determining whether or not expression of the protein is altered in the sample, thereby diagnosing or providing the prognosis for the cancer. In some embodiments, the cancer is a CD20 expressing cancer, including lymphoma. The molecular signaling pathway can be functional or activated AKT or NFκB. The protein can be PTEN, AKT, Fas, YY1, NFκB, NIK, IKK, IKB, Bcl-2, Bcl-_{XL}, AP-1 or STAT3 or other member set forth in FIGS. 5 or 11. In other embodiments, the pathway is selected from a p38 MapK/Stat 3, Raf 1/MEK 1/2/ERK 1/2, Nf-kappa B or Akt pathway. The antibody can be a monoclonal antibody. The tissue sample, in some embodiments, is one fixed or embedded in paraffin. In yet other embodiments, the tissue sample is a metastatic cancer tissue sample. The tissue sample, can be from blood, bone marrow, prostate, ovary, bone, lymph node, liver, kidney, or sites of metastases. In some embodiments of any of the above, the methods indicates whether the cancer is sensitive or resistant to rituximab or another agent (e.g., monoclonal antibody) which binds CD20.

In still another aspect, the invention provides a method of diagnosing a cancer or providing a prognosis for a cancer that that has altered expression of molecular signaling pathways triggered by rituximab, by contacting a tissue sample with a primer set of a first oligonucleotide and a second oligonucleotide that each specifically hybridize to a nucleic acid encoding a protein that is part of a molecular signaling pathway triggered by rituximab; and determining whether or not expression of the nucleic acid is altered in the sample; thereby diagnosing the cancer; amplifying YY1 nucleic acid in the sample; and determining whether or not YY1 nucleic acid is overexpressed in the sample; thereby diagnosing the cancer that overexpresses YY1. In some embodiments of any of the above, the cancer is a CD20 expressing or over-expressing cancer. In other embodiments of any of the above, the cancer is a rituximab resistant cancer.

In yet another aspect. the invention provides a method of localizing a cancer in vivo, the cancer having altered expression of molecular signaling pathways triggered by rituximab, imaging in a subject a cell a polypeptide member of the molecular signaling pathways triggered by rituximab (e.g., p38 MapK/ Stat 3, Raf 1/MEK 1/2/ERK 1/2, Nf-kappa B or Akt pathway), thereby localizing cancer in vivo. The polypeptide member in some embodiments can be PTEN, AKT, Fas, YY1, NFκB, NIK, IKK, IKB, Bcl-2, Bcl-_{XL}, AP-1 or STAT3 or other member set forth in FIGS. 5 or 11. In some embodiments the cancer is a CD20 expressing or over-expressing cancer. In some embodiments of any of the above, the cancer is a rituximab-resistant cancer.

In still other aspects, the invention provides methods of identifying a compound or agent that inhibits a cancer that has an altered molecular signaling pathways triggered by rituximab (e.g., a p38 MapK/ Stat 3, Raf 1/MEK 1/2/ERK 1/2, Nf-kappa B or Akt pathway) by contacting a cell expressing a polypeptide member of the molecular signaling pathways triggered by rituximab with a compound; and determining the effect of the compound on the polypeptide; thereby identifying a compound that inhibits the cancer. The polypeptide member in some embodiments can be PTEN, AKT, Fas, YY1, NFκB, NIK, IKK, IKB, Bcl-2, Bcl-_{XL}, AP-1 or STAT3 or other member set forth in FIGS. 5 or 11. In some embodiments the cancer is a CD20 expressing or over-expressing cancer. In some embodiments of any of the above, the cancer is a rituximab-resistant cancer. In some embodiments the cancer is a CD20 expressing or over-expressing cancer.

In a related aspect, the invention provides methods of identifying a compound that inhibits a therapy resistant cancer by contacting a cell expressing a polypeptide member of a molecular signaling pathways triggered by rituximab (see FIG. 5) with a compound; and determining the effect of the compound on the polypeptide; thereby identifying a compound as one that inhibits the therapy resistant cancer. In some embodiments the cancer is a CD20 expressing or under-expressing cancer. In some embodiments of any of the above, the cancer is a rituximab-resistant cancer.

In a further aspect, the invention provides methods of treating or inhibiting a cancer in a subject that that has an altered molecular signaling pathway triggered by rituximab by administering to the subject a therapeutically effective amount of one or more inhibitors that modulates a polypeptide member of a molecular signaling pathway triggered by rituximab(e.g., a p38 MapK/ Stat 3, Raf 1/MEK 1/2/ERK 1/2, Nf-kappa B or Akt pathway). The polypeptide member in some embodiments can be PTEN, AKT, Fas, YY1, NFκB, NIK, IKK, IKB, Bcl-2, Bcl-_{XL}, AP-1 or STAT3 or other member set forth in FIGS. 5 or 11. In some embodiments the cancer is a CD20 expressing or over-expressing cancer. In some embodiments of any of the above, the cancer is a rituximab-resistant cancer. In some embodiments the cancer is a CD20 expressing or over-expressing cancer. The inhibitor can be a small organic molecule or a chemical inhibitor. In some embodiments, the inhibitor is an NO donor. In still further embodiments, the NO donor is selected from the group consisting of L-arginine, amyl nitrite, isoamyl nitrite, nitroglycerin, isosorbide dinitrate, isosorbide-2-mononitrate, isosorbide-5-mononitrate, erythrityl tetranitrate, pentaerythritol tetranitrate, sodium nitroprusside, 3 morpholinosydnonimine, molsidomine, N-hydroxyl-L-arginine, S,S-dinitrosodthiol, ethylene glycol dinitrate, isopropyl nitrate, glyceryl-1-mononitrate, glyceryl-1,2-dinitrate, glyceryl-1,3-dinitrate, glyceryl trinitrate, butane-1,2,4-triol trinitrate, N,O diacetyl-N-hydroxy-4-chlorobenzenesulfonamide, NG hydroxy-L-arginine, hydroxyguanidine sulfate, (±)-S-nitroso-N-acetylpenicillamine, S nitrosoglutathione, (±)-(E)-ethyl-2-[(E)-hydroxyimino]-5-nitro-3-hexeneamide (FK409), (±)-N-[(E)-4-ethyl-3-[(Z)-hydroxyimino]-5-nitro-3-hexen-1-yl]-3-pyridinecarboxamide (FR144420), 4-hydroxymethyl-3-furoxancarboxamide, (Z)-1-[2-(2-Aminoethyl)-N-(2-ammonioethyl)amino]diazen-1-ium-1,2-diolate; NOC-18; 3,3-bis(aminoethyl)-1-hydroxy-2-oxo-* 1-triazene (DETA/NONOate), NO gas, and mixtures thereof. In other embodiments, the inhibitor is an siRNA or an antimitotic drug (e.g., a vinca alkaloid or taxane).

In yet other aspects, the invention provides methods of treating or inhibiting a therapy resistant cancer in a subject comprising administering to the subject a therapeutically effective amount of one or more inhibitors of a polypeptide member of a molecular signaling pathway triggered by rituximab. In an exemplary embodiment, the cancer is a rituximab resistant cancer in which the above survival pathways are hyper-activated and the inhibitors reverse or oppose the drug-resistance by modulating a pathway triggered by rituximab (see FIGS. 5., 11, and 13 to 15). The inhibitor can be a small organic molecule or a chemical inhibitor. In some embodiments, the therapy resistant cancer has altered expression of molecular signaling pathways normally triggered by rituximab, and the therapy-resistant cancer was diagnosed by contacting a tissue sample of the cancer with an antibody that specifically binds to protein that is part of a molecular signaling pathway normally triggered by rituximab; and determining whether or not expression of the protein is altered in the sample, thereby diagnosing or providing the prognosis for the cancer. In some embodiments, one or more inhibitors are administered concurrently with another cancer therapy which may or may not include rituximab.

In a still further aspect, the invention provides a method of treating or chemo-sensitizing a patient having a CD-20 expressing cancer, said method comprising administering to the patient a modulator of a p38 MapK/ Stat 3, Raf 1/MEK 1/2/ERK 1/2, Nf-kappa B or Akt pathway or other rituximab-responsive pathway(see, FIGS. 5, 11, and 13 to 15) or a polypeptide component thereof. The CD-20 expressing cancer can be selected from the group consisting of lymphoma, B-acute lymphoblastic lymphoma, non-Hodgkin's lymphoma, Burkitt's small cell, and large cell lymphomas, chronic lymphocytic leukemia, Hodgkin's lymphoma, leukemia, acute myelogenous leukemia, acute lymphoblastic leukemia, chronic modulator myelogenous leukemia, and multiple myeloma. In still further embodiments of any of the above, the modulator is a pro-apoptosis or chemosensitizing modulator of a p38 MapK/ Stat 3, Raf 1/MEK 1/2/ERK 1/2, Nf-kappa B or Akt pathway. In some embodiments, the modulator binds PTEN, AKT, Fas, YY1, NFκB, NIK, IKK, Bcl-2, Bcl-_{XL}, AP-1, STAT3 or IKB. In some further embodiments, the CD-20 expressing cancer was identified to have a hyperactive p38 MapK/ Stat 3, Raf 1/MEK 1/2/ERK 1/2, Nf-kappa B or Akt pathway. In yet other embodiments, the CD-20 expressing cancer was identified to have a hyperactive p38 MapK/Stat 3, Raf 1/MEK 1/2/ERK 1/2, Nf-kappa B or Akt pathway by determining whether or not expression or amounts of a protein of the pathway is altered. In still further embodiments, the protein can be selected from the group consisting of PTEN, AKT, Fas, YY1, NFκB, NIK, IKK, Bcl-2, Bcl-_{XL}, AP-1, STAT3, and IKB. In any embodiments of the above, rituximab may or may not also be administered. In some embodiments, the modulator binds to a marker selected from PTEN, AKT, Fas, YY1, NFκB, NIK, IKK, Bcl-2, Bcl-_{XL}, AP-1, STAT3, and IKB.

Advantageously, the invention provides methods of sensitizing cancers to chemotherapy or immunotherapy by administering modulators of a p38 MapK/ Stat 3, Raf 1/MEK 1/2/ERK 1/2, Nf-κB or Akt pathway. Those are the p38 MapK/ Stat 3, Raf 1/MEK 1/2/ERK 1/2, Nf-κB pathway and the Akt pathway. Inhibition of these pathways provides selective inhibition downstream of anti-apoptotic gene products such as Bcl-2 and Bcl-_{XL}and can result in the reversal of drug resistance and chemo sensitize cancers to various chemotherapeutic drugs. These inhibitors can mimic the effects of rituximab therapy and/or chemosensitize tumor cells. They may be administered with or without rituximab. The cancer can be CD-20 expressing cancer selected from the group consisting of lymphoma, B-acute lymphoblastic lymphoma, non-Hodgkin's lymphoma, Burkitt's small cell, and large cell lymphomas, chronic lymphocytic leukemia, Hodgkin's lymphoma, leukemia, acute myelogenous leukemia, acute lymphoblastic leukemia, chronic modulator myelogenous leukemia, and multiple myeloma. In still further embodiments of any of the above, the modulator is a pro-apoptosis or chemosensitizing modulator of a p38 MapK/ Stat 3, Raf 1/MEK 1/2/ERK 1/2, Nf-kappa B or Akt pathway. In some embodiments, the modulator binds PTEN, AKT, Fas, YY1, NFκB, NIK, IKK, Bcl-2, Bcl-_{XL}, AP-1, STAT3 or IKB. In some further embodiments, the CD-20 expressing cancer was identified to have a hyperactive p38 MapK/ Stat 3, Raf 1/MEK 1/2/ERK 1/2, Nf-kappa B or Akt pathway. In yet other embodiments, the CD-20 expressing cancer was identified to have a hyperactive p38 MapK/Stat 3, Raf 1/MEK 1/2/ERK 1/2, Nf-kappa B or Akt pathway by determining whether or not expression or amounts of a protein of the pathway is altered. In still further embodiments, the protein can be selected from the group consisting of PTEN, AKT, Fas, YY1, NFκB, NIK, IKK, Bcl-2, Bcl-_{XL}, AP-1, STAT3, and IKB. In some embodiments, the modulator binds to a marker selected from PTEN, AKT, Fas, YY1, NFκB, NIK, IKK, Bcl-2, Bcl-_{XL}, AP-1, STAT3, and IKB.

In another aspect, the invention provides a method of immunotherapy for cancer cells expressing CD20 by administering rituximab or another monoclonal antibody against CD20 with an immunotherapeutic agent. Such treatment can regulate the cancer cells' sensitivity to immunotherapy by upregulating death receptors and sensitizing the cells to Fas ligand and TRAIL-induced apoptosis. The upregulation of death receptors can result from the inhibition of the transcription repressor Ying Yang 1 (YY 1) that is itself regulated by Nf-κB. In addition, pharmacological inhibitors for NfκB or YY1 can be administered to provide a similar therapeutic action as rituximab, with or without co-administration of rituximab, in sensitizing tumor cells to immunotherapy.

### BRIEF DESCRIPTION OF THE DRAWINGS

FIG. 1. Inhibition of the PI3K/Akt signaling pathway in Ramos by rituximab: (A) Ramos cells (1×10⁷/ml) were treated with rituximab (20 μg/ml) for different times (0-24 h) and incubated at 37° and cell lysates were prepared as described in methods. The cell lysates were examined by western for various unphosphorylated and phosphorylated (p) proteins of the Akt pathway. B-actin was used as control for loading. (B) Inhibition of the PI3K/Akt signaling pathway in Ramos by rituximab: Ramos cells were treated with rituximab (138 nM) or with equal amount of rituximab (Fab′)₂ (78.3 nμ) for 20 h at 37° and cell lysates were prepared and examined for Akt and p-Akt (ser473). B-actin was used as control. (C) Ramos cell lysates were prepared as described above in A. and examined by western for unphosphorylated and phosphorylated proteins of the NF-κB pathway. B-actin was used as control. The above findings are representative of three independent experiments yielding similar results.

FIG. 2. Rituximab-mediated augmentation of the association of Bad with Bcl-_{XL} to form heterodimeric complexes: (A) Ramos cells (2×10⁶/ml) were left untreated or were treated with rituximab (20 μg/ml) for 20 h and cell lysates were prepared. The cell lysates were immunoprecipitated with Rabbit anti-Bcl-_{XL} antibody and the precipitate was examined by western for Bad using Rabbit anti-Bad antibody as described in Methods. The IgG bands in this figure correspond to the immunoprecipitated rabbit anti-Bcl-_{XL} IgG and its development by the secondary goat anti-rabbit IgG and then developed with HRP-goat anti-rabbit IgG. (B) Ramos cells were treated with rituximab (20 μg/ml), the Akt inhibitor LY294002 (25 μm) or with the combination for 24 h. The cell lysates were prepared and examined by western for Bcl-_{XL} levels. B-actin was used as control. The above findings are representative of three independent experiments yielding similar results.

FIG. 3. Chemosensitization of Ramos and Ramos RR1 by rituximab, rituximab (Fab′)₂ and by LY294002: (A) Ramos cells were treated with different concentrations of rituximab for 20 h and then treated with CDDP (15 μg/ml) for an additional 24 h. The cells were then evaluated for apoptosis by the PI method as described in Methods. p<0.001 represents the combination treatment of rituximab (0.138 μm) and CDDP as compared to treatment with CDDP alone. p<0.005 represents the combination treatment of rituximab (0.104 μM)+CDDP as compared to treatment with CDDP alone. (B) Similar experiments to those described in A. above were performed except that rituximab (Fab′)₂ was used instead of rituximab. p<0.001 represents the combination of rituximab (Fab′)₂ plus CDDP as compared to treatment with CDDP alone. (C) Similar experiments as those described above in A. were performed except that Ly294002 was used instead of rituximab. p<0.005 represents the combination of LY294002 (20 and 30 μM) and CDDP as compared to treatment with CDDP alone. (D) Similar experiments as those described above in (C) were preformed except that adriamycin (ADR) was used instead of CDDP. p<0.001 represents the combination of LY294002 (10 and 25 μM) plus ADR (5 μg/ml) as compared to treatment with ADR alone. (E) The rituximab resistant Ramos clone, RR1, was treated as described above in (A). for Ramos wild type. (F) The rituximab resistant Ramos clone, Ramos RR1, was treated as described above in (C). for Ramos wild type. p<0.005 represent the combination of LY294002 (60 μM) and CDDP (15 μg/ml) as compared to treatment with CDDP alone. The above findings are representative of three independent experiments yielding similar results.

FIG. 4. Direct role of the Akt pathway in chemosensitization of Ramos to CDDP-induced apoptosis: (A) Ramos cells were transfected with control siRNA or Akt siRNA for different periods of time (0-72 h) as described in methods. Cell lysates were prepared and examined by western for p-Akt, Akt and Bcl-_{XL} expression. (B) Ramos cells were transfected with control siRNA or different concentrations of Akt siRNA for 48 h. The cells were then treated with CDDP (15 μg/ml) for an additional 25 h and the cells were examined for apoptosis as described. p<0.001 represent the combinations of Ramos cells transfected with Akt siRNA (5, 10, and 20 μM) and CDDP as compared to treatment with CDDP alone. The above findings are representative of three independent experiments yielding similar results.

FIG. 5. Schematic diagram of rituximab-mediated inhibition of the Akt pathway and chemosensitization: This diagram shows that Ramos cells exhibit constitutively activated Akt and NF-κB pathways and these are represented in dotted lines. Activation of these pathways result downstream in cytosolic Bcl-_{XL} that is not complexed with p-Bad and overexpression of Bcl-_{XL} leading to chemoresistance. In contrast, treatment with rituximab inhibits these pathways resulting in augmentation of association of Bcl-_{XL} and Bad as well as downregulation of Bcl-_{XL} expression leading to chemosensitization.

FIG. 6. (A) Surface expression of CD20 on RR1 clones. Cells (2×10⁶) were stained with 1 μg anti-CD20 mAb (IgG1 subtype; solid black lines) or isotype control (pure IgG1; gray lines) and subsequently with FITC-labeled secondary Abs and analyzed by FACS. The intensity of surface CD20 expression was measured by the mean fluorescence intensity (MFI) (n=3). (B) Rituximab inhibits the proliferation of the wt but not the RR clones. Cells were left either untreated or treated with rituximab (20 μg/ml-24 h) and (10⁴ cells in triplicates) were used in standard XTT assay. (C) An aliquot of the above samples were examined microscopically to assess the ability of rituximab to form homotypic aggregation of the clones. (D) Failure of cross-linked rituximab to induce apoptosis in RR clones. Cells (2×10⁶) were either left untreated or treated with anti-hIg, rituximab or the optimal concentration of cross-linked rituximab (50 μg/ml anti-hIg+20 μg/ml rituximab-24 h) and subjected to PI assay for the percentage of apoptotic cells. (E) Failure of rituximab to mediate CDC in the RR clones. Cells (2×10⁶) were left either untreated or treated with human Ab serum (5% and 10%), rituximab or the combination for 24 h and subjected to PI staining (without fixing) for the percentage of dead cells. (F) Failure of rituximab to chemo-sensitize the RR clones. Cells (2×10⁶) were left either untreated or pre-treated with rituximab (20 μg/ml-24 h). Then, the cells were washed, and were grown in complete medium supplemented with paclitaxel (0.1-10 nM-18 h). Then, the cells were stained with PI solution and apoptosis was assessed by FACS. Samples were set up in duplicates and the results are represented as the mean±SD (n=2). *P values <0.05 significant compared to control.

FIG. 7. Higher drug-resistance and over-expression of the anti-apoptotic Bcl-2 family members in the RR clones. A. Cells (2×10⁶) were left either untreated or treated with various concentrations of paclitaxel (1.0, 10, 20 μg/ml), ADR (0.5, 1.0, 2.0 μg/ml), CDDP (1.0, 10, 20 μg/ml), vincristine (0.1, 0.5, 1.0 μg/ml), and VP-16 (1.0, 10, 20 μg/ml) for 18h. Then, the cells were stained with PI solution and apoptosis was assessed by FACS. Samples were set up in duplicates and the results are represented as the mean±SD (n=2). (Fold drug-resistance was measured using the highest percentage of apoptosis induced by the highest concentration of the drug in the wt cells as 100% and calculating the required drug concentration to achieve the same level of apoptosis in the clones) B. Total RNA of various culture conditions was extracted from 10⁷ cells (as indicated) and converted to cDNA. 2.5 μg cDNA was used in qPCR analysis to determine the transcripts levels. Levels of G-3-PDH were confirmed for equal loading. Results are represented as mean±SD of triplicate samples. C. WCEs (40 μg) of wt cells and RR clones (±20 μg/ml rituximab-24 h) were subjected to immunoblotting for protein levels. Levels of β-actin were used for equal loading (n=2). *P values <0.05 significant compared to control.

FIG. 8. Hyper-activation of the NF-κB and ERK1/2 survival pathways in the RR clones. After overnight growth in RPM11640+1% FBS, RR clones were washed and grown in complete medium±rituximab (20 μg/ml; 24 h). A. Total cell lysates (40 μg) were subjected to western blot analysis using phospho-specific Abs for various components of the NF-κB and ERK1/2 pathways. B. The kinase activity of the IKK complex using IκB-α peptide (aa 1-50 including S^{32/36}) and ERK1/2 using MAPK kinase substrates 4 (aa 172-192) using immune-complex kinase assay. C. After overnight growth in RPMI1640+1% FBS, cells were washed and were grown in complete medium (±rituximab, DHMEQ, or PD098059). 10 μg of nuclear lysates were subjected to EMSA (n=2) (19, 20).

FIG. 9. Chemo-sensitization of the RR clones by chemical inhibitors. Cells (2×10⁶) were left either untreated or pretreated with A. DHMEQ (wt: 10 μg/ml, clones: 20 μg/ml), B. bortezomib (wt: 4 μM, clones: 8 μM) or C. PD098059 (wt: 15 μg/ml, clones: 30 μg/ml) for 2 h. Cells were then incubated with paclitaxel (1.0, 10, 20 μg/ml), ADR (0.5, 1.0, 2.0 μg/ml), CDDP (1.0, 10, 20 μg/ml), vincristine (0.1, 0.5, 1.0 μg/ml), and VP-16 (1.0, 10, 20 μg/ml) for an additional 18h and subjected to DNA fragmentation assay. The samples were set up in duplicates and the results are represented as the mean±SD of two independent experiments. *P values <0.05 significant compared to drug treatment alone.

FIG. 10. Inhibition of the expression of anti-apoptotic factors by chemical inhibitors. RR1 clones were left either untreated or treated with DHMEQ (20 μg/ml), bortezomib (8.0 μM) and PD098059 (30 μg/ml). A. 2.5 μg cDNA was used in qPCR using gene specific primers. Levels of G-3-PDH were confirmed for equal loading. Samples were set up in duplicates and the results are represented as the mean±SD (n=2). B. Total cell lysates (40 μg) were subjected to immunoblotting for Bcl-2, Bcl-_{XL} and Mcl-1. Levels of β-actin were used for equal loading (n=2). C. Role of anti-apoptotic Bcl-2 members in chemo-sensitization. Cells were either left untreated or pretreated with 2MAM-A3 (wt: 15 μg/ml, clones: 35 μg/ml-7 h). The cells were then washed, treated with paclitaxel (10 nM-18 h) and subjected to DNA fragmentation assay. Samples were set up in duplicates and results are represented as mean±SD of two independent experiments. *P values <0.05 significant compared to paclitaxel alone.

FIG. 11. Proposed model of the development of RR in NHL B-cells. Among other mechanisms (18) alterations in the dynamics of cell signaling, via an elusive mechanism, is a potential mechanism for the development of resistance to rituximab therapy. Continuous long-term rituximab exposure results in the development of NHL B-clones that exhibit diminished CD20 surface expression. Compared to the parental cells, the clones exhibit significantly lower sensitivity to CDC-mediated killing, apoptosis induced by cross-linked rituximab and higher levels of p-IκB-α, p-IKK, p-ERK1/2 correlating with higher IKK and ERK1/2 kinase activities leading to higher DBA of AP-1 and NF-κB culminating in increased Bcl-2, Mcl-1, Bcl-_{XL} expression. Hyper-activation of these pathways will a) increase the proliferation rate of the clones, b) increase the levels of Bcl-2, Bcl-_{XL}, Mcl-1 and the apoptosis threshold, and c) cause higher chemo-resistance of the NHL B-clones. Specific pharmacological inhibition of NF-κB and ERk1/2 pathways, or functional impairment of anti-apoptotic Bcl-2 family members avert the acquired chemo-resistance phenotype of the RR-clones inducing the cells to undergo apoptosis in response to low levels of drugs.

FIG. 12. Chemosensitization of the RR clones by chemical inhibitors. (A) RR clones (2×10⁶/treatment) were left untreated or pretreated with DHMEQ (20 μg/ml) bortezomib (8 μM), or PD98059 (30 μg/ml) for 2 hours. Cells were incubated with paclitaxel (20 μg/ml). ADR (2.0 μg/ml), CDD_(20 μg/ml) vincristine (1.0 μg/ml) and VP-16 for an additional 18 hours and subjected to DNA fragmentation assay. Samples were set in duplicates and the results represented as mean+/−SD of two independent experiments. In all cases, statistically significant values ( P<0.05) were obtained by the combination compared to drug and/or inhibitor treatment alone. (B). Fold enhancement of apoptosis by treatment of RR clones with inhibitors based on data from part A.

FIG. 13. Roles of p38MAPK and STAT3 in the regulation of Bcl-2 and chemoresistance in the 2F7 AIDS-derived DLBCL cell line. This schematic diagram represents the effect of rituximab treatment on inhibiting the activity of p38MAPK, NF-κB, SP1, IL-10 transcription and expression, IL-10-IL-10R signaling and STAT3 activity leading to downregulation of Bcl-2 and chemosensitization of drug-resistant NHL cells.

FIG. 14. Riruximab-mediated inhibition of the ERK1/2, NF-κB and AKT survival pathways in the Ramos and Daudi B-NHL cell lines. This schematic diagram shows that rituximab inhibits the Raf-1/Mek1/2/ERK1/2, NF-κB, and AKT signaling pathways all leading to downregulation of the transcription and activity of Bcl-xl and chemosensitization. In addition, rituximab induces RKIP expression which participates in the inhibition of both the ERK1/2 and NFkB pathways. The rituximab resistant clones failed to respond to rituximab treatment and no cell signaling was achieved and therefore the tumor cell remained chemoresistant.

FIG. 15. Rituximab-mediated upregulation of death receptors and sensitization to FasL and TRAIL-induced apoptosis in the 2F7, Daudi and Ramos cell lines. This schematic diagram shows that rituximab inhibits NF-κB and YY1 activities leading to inhibition of the repressor activity of YY1 on both Fas and DR5 transcription and expression. This results in the upregulation of these death receptors and sensitization of NHL cells to FasL and TRAIL-induced apoptosis. In addition, pharmacologic inhibitors of NF-κB and YY1 mimics rituximab and sensitizes the cells to FasL and TRAIL-induced apoptosis.

### DETAILED DESCRIPTION OF THE INVENTION

The invention relates to the Applicants' discovery that rituximab signals the lymphoma cells and inhibits several intracellular signaling pathways (e.g., the p38 MapK/Stat 3, Raf 1/MEK1/2/ERK 1/2, NfκB pathway and the Akt pathways) and that pharmacological inhibitors of those various pathways can mimic rituximab and chemosensitize tumor cells. Inhibition of these pathways resulted in the selective inhibition downstream of anti-apoptotic gene products such as Bcl-2 and Bcl-_{XL} that resulted in the reversal of drug resistance and chemo sensitized the cells to various chemotherapeutic drugs. The invention also relates to the Applicants' discoveries that upon the development of rituximab resistance in their rituximab-resistant clones, the above survival pathways are hyper-activated and that pharmacological inhibitors of these pathways could reverse the drug-resistance. Additionally, the Applicants have validated the cell lines examined herein by demonstrating that in patients with non-Hodgkin's Lymphoma the above signaling pathways are hyper-activated in the examined tumor tissues.

The invention also relates to the Applicants' discovery that rituximab treatment regulates the tumor cells sensitivity to immunotherapy. The treatment resulted in upregulation of death receptors and sensitization to Fas ligand and TRAIL-induced apoptosis. The upregulation of death receptors by rituximab was the result of the inhibition of the transcription repressor Ying Yang 1 that is itself regulated by Nf-κB. In addition, pharmacological inhibitors for NfκB or YY1 were found to mimic rituximab and sensitize the tumor cells to immunotherapy. Accordingly, the modulators of the survival pathways are particularly useful in sensitizing a patient's cancer cells to both chemotherapy and immunotherapy.

In particular, with respect to rituximab-mediated effects, [0040] the following have been shown to be inhibited by rituximab:
Src kinases; p38 MAPK/STAT-3/IL-10/Bcl-2; Ras/Raf-1/MEK1/2/ERK1/2/Bcl-x; NIK/IKK/IKB/TAK-1 /Bcl-x; PI3K/PDK1 /AKT/Bad/Bcl-xL; NFκB/YY1

In particular, with respect to rituximab-mediated effects, the following was shown to be induced by rituximab:
RKIP, PTEN, Fas, DR5, Bad

In particular, with respect to rituximab-mediated effects, the following transcription factors were shown to be inhibited by rituximab:
NFκB, AP-1, SP-1, STAT-3, YY1

Additionally, the following agents have been shown to modulate markers and pathways.

TABLE 2
Sensitizing agents for drug-resistant B-NHL cell lines

| Sensitizing | | Modified gene | Chemotherapeutic | |
|---|---|---|---|---|
| Agent | B-NHL cell line | product(s) | drug(s) | Reference |
| Rituximab | 2F7 DLBCL, | Bcl-2 | CDDP, | (Alas, S. et al., |
| | 10C9 | | Fludarabine, ADR, | Clinical Cancer |
| | | | Vinblastine | Research, 7: |
| | | | | 709-23 (2001)) |
| Piceatannol | 2F7 DLBCL | Bcl-2 | CDDP, | (Alas, S. A. et |
| | | | Fludarabine, ADR, | al., Clin. Cancer |
| | | | Vinblastine | Res. 9: 316-26 |
| | | | | (2003)) |
| Rituximab | Raji, Daudi, | Bcl-2, Bcl-_{xL} | Paclitaxel, | (Emmanouilides, |
| | Ramos, 2F7 | | Gemcitabine, | C. et al., Cancer |
| | | | Vinorelbine | Biother and |
| | | | | Radiopharm 17: |
| | | | | 621-630 (2002)) |
| Rituximab | Ramos, 2F7 | Bcl-2, Bcl-_{xL,} | Paclitaxel, ADR, | (Jazirehi, A. R. |
| | | Apaf-1 | CDDP, Vincristine, | et al., Molecular |
| | | | VP-16 | Cancer Therapy, 2: |
| | | | | 1183-93 (2003)) |
| Rituximab, | 2F7 | p38 MAPK, NF-κB, | CDDP | (Vega, M. I. et |
| PP2, SB 203580, | | Sp-1, STAT-3, | | al., Oncogene, 23: |
| Bay 11-7085 | | IL-10, Bcl-2 | | 3530-40 (2004)) |
| Rituximab, GW | Ramos, Daudi | Raf-1/MEK1/2/ | Paclitaxel, ADR, | (Jazirehi, A. R. |
| 5074, PD- | | ERK1/2, AP-1, | CDDP, Vincristine, | et al., Cancer |
| 048059, UO126, | | Bcl-_{xL}, RKIP | VP-16 | Research, 64: |
| 2MAM-A3 | | | | 117-26 (2004)) |
| Rituximab, | Ramos, Daudi | p-NIK, IKK, IκB-α, | Paclitaxel, ADR, | (Jazirehi, A. R. |
| Bay 11-7085, | | NF-κB, RKIP | CDDP, Vincristine, | et al., Cancer |
| DHMEQ, SN-50, | | | VP = 16 | Research 65: |
| 2-MAM-A3 | | | | 264-276 (2005)) |
| Rituximab | Ramos | PI3K, PDK1, AKT, | CDDP | (Hongo, F. et al., |
| | | IKK, Bad, Bcl-_{xL}, | | Biochemistry |
| | | siRNA AKT | | and Biophysical |
| | | | | Research |
| | | | | Communications 336: |
| | | | | 692-701 (2005)) |

A number of approved drugs and antibodies can also interfere with those pathways and those include: Gleevec, Bortezomib, Trastuzumab, cetuximab/erbitux, gemtuzumab, doxil, gefitinib, roferonA/intron-A, nitromed (NO donor for cardiovascular diseases), prokine, avastin, campath and various chemotherapeutic drugs.

Accordingly, the above findings are of clear-cut prognostic, diagnostic and therapeutic significance. Additionally, upregulation of the death receptor by rituximab as well as the response of these pathways to cytotoxic immunotherapy are significant diagnostic/prognostic/and therapeutic targets.

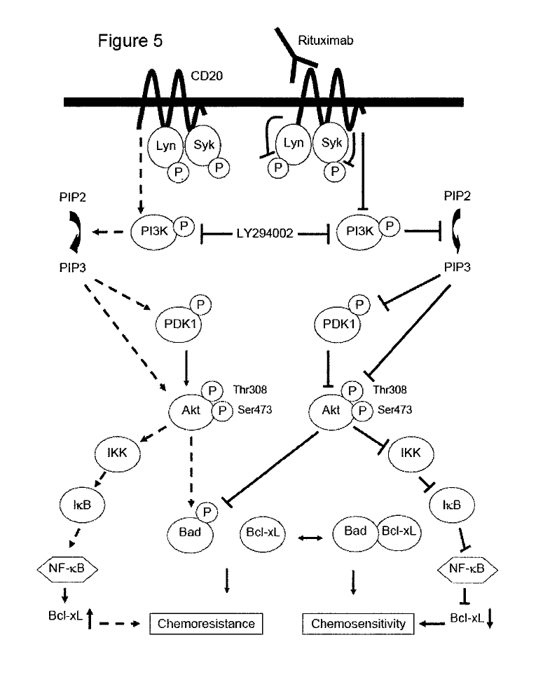

Accordingly, this invention identifies molecular signaling pathways (e.g., p38 MapK/ Stat 3, Raf 1/MEK 1/2/ERK 1/2, Nf-κB pathway and the Akt pathways, and see FIG. 5, 11, 13 to 15) that can be modified following rituximab treatment of CD-20 expressing cancers such as NHL and that can segregate patients to treatment with respect to therapy with rituximab alone or in combination with chemotherapy. These pathways modified by rituximab are intrinsically involved in the regulation of drug resistance and identify several targets of therapeutic, prognostic and diagnostic significance that are members of the signaling pathways, such as functional or activated Bcl-2/Bcl-_{XL}, AKT, PTEN, Fas, YY1, NFκB, NIK, IKK, IKB, and transcription factors AP-1 and STAT3. One way of practicing the invention is to examine patients' tumor cells for the activation state of these signaling pathways so as to determine the probability of whether they would be modified by rituximab therapy alone or in combination with chemotherapy. For example, patients' tumors that show hyper-activation of one or more than one of these pathways and/or may have deficiencies in any of the members of these pathways will be considered unlikely to respond to treatment. Such patients will need a different therapeutic approach and the use of either different drugs and/or agents that can normalize the activity of the signaling pathways to respond to treatment. Likewise, patients who become refractory to treatment will also benefit from examining the tumor cells for the status of these signaling pathways and their components, such as functional or activated Bcl-2/Bcl-_{XL}, AKT, PTEN, Fas, YY1, NFκB, NIK, IKK, IKB, and transcription factors AP-1 and STAT3 and determine best courses for intervention. In addition, patients with recurrences may be screened for the molecular signature of these pathways (e.g., Bcl-2/Bcl-_{XL}, AKT, PTEN, Fas, YY1, NFκB, NIK, IKK, IKB, and transcription factors AP-1 and STAT3) and then treated accordingly or by the corresponding suitable treatment. The various analyses of the signaling pathways may utilize tumor tissue for immunohistochemistry protein expression by Western, transcription factor activity by EMSA, transcripts by RT-PCR, microarray analysis, proteomics, etc. In addition to the diagnostic/prognostic invention, this invention has important therapeutic application and can be used to screen for agents that can selectively modify aberrant abnormal signaling pathways (functional or activated Bcl-2/Bcl-_{XL}, AKT, PTEN, Fas, YY1, NFκB, NIK, IKK, IKB, and transcription factors AP-1 and STAT3) in the cancer cells and that can then be used directly or in combination with chemotherapy using conventional drugs.

This invention describes several signaling pathways and their components (e.g., p38 MAPK pathway (SP1, STAT3, NFκB); src/Raf1/ERK1/2 (MEK1/2, AP-1, Bcl-2/Bcl-_{XL},RKIP): NFκB (NIK, IKK, IKB); AKT (PI3K, PDBK1, Bad, Bcl-_{XL}, PTEN) (see FIG. 5 also) triggered in NHL tumor cells following treatment with rituximab which result in the modification of the activity of specific cell signaling pathways that change the tumor cells behavior and cell growth characteristics as well as reducing the threshold for resistance to apoptotic stimuli. The pathways that have been uncovered in this invention include the Raf-1/MEK1/2/ERK1/2 and NF-κB, PTEN, P38MAPK signaling pathways where rituximab treatment results in inhibition of their activity. Further, this invention also describes a mechanism by which rituximab is able to down regulate these pathways.

In one study (Jazirehi, A. et al., Cancer Res. 64(19):7117-26 (2004)) we demonstrate that treatment of B-NHL cell lines with rituximab inhibited the kinase activity of mitogen activated protein kinase (MEKK1/2) and reduced the phosphorylation of the components of the ERK1/2 pathway (Raf-1/MEKK1/2/ERK1/2) and decreased activation protein 1 (AP-1) DNA-binding activity resulting in selective downregulation of the anti-apoptotic gene product Bcl-_{XL}. These above events occur with similar kinetics and were observed 3 to 6 h following rituximab treatment. Rituximab mediated affects were corroborated with specific pharmacological inhibitors. In addition, the inhibition of the Raf-1/MEKK1/2/ERK1/2 pathway resulted in sensitization of the drug resistant tumor cells to chemotherapeutic drug-induced apoptosis. In addition, we demonstrated that rituximab induced the expression of Raf-1 kinase inhibitor protein (RKIP) which was involved in the inhibition of the ERK1/2 signaling pathway. These novel findings revealed a signaling pathway modified by rituximab through the induction of RKIP expression which adversely regulates the activity of the ERK1/2 pathway and which regulates of Bcl-_{XL} expression and subsequently chemosensitization of drug refractory NHL tumor cells. In addition, this study identifies several potential targets that can be used for screening of new therapeutics and/or to modify their expression in patients who are unresponsive to current treatments. In addition, the pathways identified and modified by rituximab may be used to identify patients tumor cells who might not respond to rituximab treatment based on the level of expression and/or activity.

In subsequent studies (Jazirehi, A. et al., Cancer Res. 65(1):264-76 (2005); Jazirehi et al., Abstract #288, Blood, 104(11) (2004); Vega et al., Oncogene, 1-14 (2002); Vega et al., J. Immunol., 175(4):2174-2183 (2005); Jazirehi et al., Cancer Res., 65(1):264-276 (2005), herein incorporated by reference in their entirety) we have reported the second novel signaling pathway triggered by rituximab and modified following treatment of NHL cell lines with rituximab. We demonstrated rituximab inhibits the constitutively activated NF-κB activity and the NFκB signaling pathway (including components functional or activated Bcl-2/Bcl-_{XL}, Fas, YY1, NFκB, NIK, IKK, IKB, and transcription factors AP-1 and STAT3) leading to NFκB. Rituximab decreased the phosphorylation of NFκB-inducing kinase, IκB kinase, and IκBα and diminished IKK kinase activity and decreased NFκB DNA-binding activity. These events occurred rapidly following rituximab treatment (3 to 6 h). Rituximab induced Raf kinase inhibitor protein up-regulation was in part responsible for interrupting the NFκB signaling pathway and concomitant with down regulation of the anti-apoptotic gene products Bcl-_{XL} and Bfl/A1. Rituximab-mediated decreases in the expression levels of Bfl-XL , Bfl/A1 were responsible for rituximab mediated sensitization of drug resistance NHL cells to various chemical therapeutic drugs-induced apoptosis. Therefore, similar to the above findings, for the Raf 1/MEK1/2/ERK1/2 signaling pathway (modified by rituximab), the rituximab mediated inhibition of the NFκB signaling pathway is also responsible for patient response to rituximab treatment. In addition, it also identifies a subset of patients whose pathway is overactive and could not respond to rituximab treatment alone or in combination with drugs.

Detection of members of the functional or activated a p38 MapK/ Stat 3, Raf 1/MEK 1/2/ERK 1/2, NfκB or Akt molecular signaling pathways, such as functional or activated Bcl-2/Bcl-_{XL}, AKT, PTEN, Fas, YY1, NFκB, NIK, IKK, IKB, and transcription factors AP-1 and STAT3 is therefore useful for diagnosis and prognosis of NHL as well as other CD-20 expressing cancers, such as B-acute lymphoblastic lymphoma (B-ALL), non-Hodgkin's lymphomas (e.g., Burkitt's, Small Cell, and Large Cell lymphomas), chronic lymphocytic leukemia, and Hodgkin's lymphoma, and multiple myeloma, as well as for B cell mediated diseases such as autoimmune diseases, rheumatoid arthritis, lupus, transplantation. Detection can include, for example, the level of mRNA or protein expression, or the localization (i.e., in the nucleus or the cytoplasm) of mRNA or protein. In terms of early diagnosis, needle, surgical or bone marrow biopsies can be used and examined by immunohistochemistry for expression in cytosol or nuclei, alone or in combination with other markers such as p53, usually negative in prostate cancer and other cancers. Thus, these markers are a new positive stain that complements the traditional negative stain to enhance the diagnosis of cancers. In addition, microlaser microdissection can be used to isolate a few cells and perform RT-PCR for nucleic acid detection. Molecular imaging can be used to identify individual cells or groups of cells expressing specific proteins (functional or activated Bcl-2/Bcl-_{XL}, AKT, PTEN, Fas, YY1, NFκB, NIK, IKK, IKB, and transcription factors AP-1 and STAT3) or enzymatic activity in real time in living patients (Louie et al., 2002). The value of imaging systematically provides value for detection of metastatic cancer. Finally, cells expressing functional or activated Bcl-2/Bcl-_{XL}, AKT, PTEN, Fas, YY1, NFκB, NIK, IKK, IKB, and transcription factors AP-1 and STAT3can be used for drug discovery to identify new drugs to treat cancers, as well as to evaluate cancer treatments. Such drugs can be directly used alone or in combination with chemotherapy, immunotherapy, radiotherapy, or hormonal therapy to treat cancers that are resistant to therapy.

The invention provides methods for assaying for therapeutic agents that inhibit of the p38 MapK/ Stat 3, Raf 1/MEK 1/2/ERK 1/2, Nf-κB or Akt pathway signaling pathways, e.g., NF-KB inducing kinase; IkB kinase (IKK); and IkB alpha. Methods of inhibiting include decrease of NFkB DNA binding activity or a decrease of Bcl-_{XL} expression. The invention also provides methods of identifying drug resistant tumors and chemosensitization drugs. For example, inhibition of the NFkB pathway or Bcl-_{XL} expression/activity sensitizes drug resistant tumor cells to chemotherapy-induced apoptosis. Prognostic/diagnostic markers include hyperactivation of the components of the NFkB pathway and overexpression of Bcl-_{XL}. These correlate with tumor progression and unresponsiveness to conventional therapeutics.

The invention provides therapeutic methods, including use of rituximab in combination with immunotherapy: for example 1) agents that activate the immune system to upregulate Fas L expression will augment tumor cell response following rituximab treatment. 2) administration of Fas ligand agonists or mimetics 3) administration of antibody to Fas with no tissue toxicity 4) fusion of rituximab with FasL or FasL like entity. The invention also provides means of identifying agents that can inhibit YY1 expression/activity, such as antisense, siRNA, small molecules, and NO donors. Inhibitors of pathways that regulate YY1 will upregulate Fas on tumor cells and sensitize them to host immune cells (without activation or following activation with cytokines like interferon or IL2). Novel therapeutics include a combination of rituximab and cytokines that activate NK cells and upregulates surface FasL expression on NK cells and enhance NK killing of the tumor cells. Inhibitors of the NF-κB pathways also sensitize cells to Fas ligand, TRAIL, or agonistic monoclonal antibodies to DR4 and DR5. Rituximab resistant NHL may be treated with combination of agents that activate FasL on host immune cells, alone or in combination with low doses of sensitizing agents. All of the above for Fas-FasL can be applied for TRAIL on effector cells and death receptors on tumor cells. Data shows the roles of NFkB and YY2 in negatively regulating TRAIL death receptors and resistance to TRAIL. Agents that can regulate death receptors can be used on rituximab sensitive and rituximab resistant tumor cells whereby the host immune cells will kill the tumors. In addition, agents that upregulate FasL or TRAIL on host cells can be used in combination. Resistance to rituximab can be due in part to failure of host FasL/TRAIL to kill the tumor cells due to defect in the expression of the receptors and their failure to be upregulated by rituximab. For diagnostics and prognostics, one can examine expression of FasL/TRAIL death receptors on tumor cells. Low levels correlate with poor prognosis and may indicate high risk for unresponsiveness to rituximab treatment. One can also examine response to rituximab treatment for upregulation of death receptors. If not, the prognosis is bad. One can also test for overexpression/activity of YY1 in tumors. High levels correlates with poor prognosis. One can test for sensitization to FasL/TRAIL-induced apoptosis following rituximab treatment—failure to respond indicates a poor prognosis/diagnosis. One can test for circulating levels of death receptors prior or following treatment. High levels show diagnostic/prognostic significance. Finally, one can test for mutation of death receptors.

The present invention also demonstrates for the first time that the mechanism underlying resistance of B-NHL to chemotherapy may be distinguished from the mechanism of resistance to death receptors. This translates in patients who are resistant to chemotherapy or rituximab-mediated chemo-sensitization which may be treated with immunotherapy using rituximab in combination with immunomodulators of host immune system (e.g., natural killer cells and macrophages are effector cells that can mediate ADCC.). The present invention also identifies the NFkB pathway and Bcl-_{XL} expression responsible for chemoresistance and YY1 as responsible for immune resistance. Inhibitors of NFkB pathway will sensitize cells to both drug and immune mediated apoptosis. Inhibitors of NFkB in combination with drugs/immunotherapy will recruit host immune system to kill tumor cells. Clinical trial are being tested with combination of rituximab and IL-2. The rationale is to activate NK mediated ADCC. However, we provide other mechanisms by which activated NK cells kill tumor cells by signaling death receptors on the tumor cells. Overexpression of Bcl-_{XL} indicates chemoresistance and immune resistance. Overexpression or hyperactivation of the NFkB pathway indicates resistance to both chemo and immunotherapies. Overexpression of YY1 indicates immune resistance. High levels of Bcl-xL/Bcl2 indicates poor prognosis. Acccordingly, the expression levels of death receptors on tumor cells can indicate failure of immunotherapy and a bad prognosis.

The present invention also discloses that rituximab inhibits the functional or activated AKT signaling pathway. Inhibition of the functional or activated AKT pathways sensitizes cells to drug-induced apoptosis. This provides the establishment of new targets for intervention to reverse drug resistance. The invention also provides drugs used clinically or in clinical trials that are targeted at the functional or activated AKT pathway to act either in combination with rituximab or in combination with other chemotherapeutic drugs. Rituximab induces PTEN expression (inhibitor of the AKT pathway). Agents that activate PTEN can be used in the treatment of rituximab resistant NHL, alone or in combination with conventional or experimental therapeutics. Rituximab inhibits phosphorylation of Bad and increases the Bad-Bcl-_{XL} complex formation, thus reducing free Bcl-_{XL}, to act as anti apoptotic and a chemoresistance factor. The invention therefore provides agents that can mimic rituximab in combination with conventional therapeutics—development of molecules that can inhibit phosphoBad or enhance binding of Bcl-_{XL} with Bad. Activation of the AKT pathway indicates a bad prognosis and can also be determined by single analyses or microarray analysis. Once can also determine the levels of PTEN expression: which, if low, indicates a bad prognosis and resistance to therapies. Circulating levels of members of the activated AKT pathway or PTEN may predict recurrences or prognostic markers.

Cell Signaling and Chemosensitization

Our studies investigating the molecular signaling pathways triggered by rituximab have resulted in the demonstration of rituximab-mediated inhibition of the p38 MAPK, NFκB, ERK1/2, and AKT signaling pathways. Inhibition of these pathways resulted in the selective inhibition of Bcl-2/Bcl-_{XL} expression leading to chemosensitization and the reversal of drug resistance. Each of the above signaling pathways affected by rituximab is described briefly below.

a. Rituximab-mediated Inhibition of the p38 MAPK Signaling Pathway

Using the DLBCL AIDS-derived B-NHL cell line 2F7 as a model, which secretes cytokines such as TNFα and IL-10, we demonstrate that rituximab disrupted selectively the IL-10 autocrine/paracrine loop and this correlated with both downregulation of Bcl2 expression and chemosensitization. Rituximab treatment did not inhibit other apoptotic regulatory proteins such as Bcl-xL, BAD, p53, c-myc and latent membrane protein 1 (LMP1) (Alas, S. et al., Clinical Cancer Research, 7:709-23 (2001)). The signaling pathway by which rituximab decreases the transcription and secretion of IL-10 in 2F7 was examined. We showed that rituximab-mediated inhibition of IL-10 secretion resulted in downregulation of constitutive STAT3 activity seen in those cells (through IL-10-IL-10R interaction) and STAT3 inhibition resulted in inhibition of Bcl2 transcription and expression. The direct role by which rituximab-induced inhibition of STAT-3 activity and Bcl2 expression was corroborated by the use of a STAT 3 inhibitor piceatannol (shown to inhibit the JAK1/Tyk-2-dependent STAT-3 and STAT-5 signaling pathways) (Su, L. et al., The Journal of Biological Chemistry, 275:12661-6 (2000)). Many studies have reported that IL-10 is increased in the serum of many NHL patients and that this increase correlates to a lower rate of survival (Blay, J. Y. et al., Blood, 82:2169-74 (1993)). These studies suggest a novel therapeutic strategy aiming at interfering with IL-10 synthesis and secretion via inhibitors of the p38 MAPK/STAT-3 pathways.

In further studies, we examined the early events underlying the molecular mechanism by which rituximab inhibits IL-10 transcription and secretion and resulting in inhibition of STAT-3 activity and Bcl-2 expression. We show that rituximab signals the 2F7 cells through the p38 MAPK pathway and results in the inhibition of IL-1 0 transcription and secretion. Rituximab inhibited the constitutive activity of Lyn and p38MAPK activities resulting in inhibition of IL-10 transcription via inhibition of SP-1. The role of p38MAPK in the regulation of IL-10 was corroborated by the use of specific pharmacologic inhibitors of the p38MAPK pathway and implicating the roles of Src kinases and NF-κB. Rituximab-mediated inhibition of MAPK activity and IL-10 transcription correlated with the inhibition of both STAT-3 activity and Bcl-2 expression and resulted in drug-induced apoptosis.

We further examined the mechanism by which rituximab inhibits Bcl-2 expression and sensitizes cells to drug-induced apoptosis in 2F7 cells. We demonstrate that rituximab selectively inhibits Bcl-2 expression with no effect on other examined proteins. Treatment with CDDP induced the generation of mitochondrial reactive oxygen species, specifically intra-cellular peroxides. The combination of rituximab and CDDP acted synergistically to induce apoptosis and mitochondrial mediated apoptotic events (Alas, S. et al., Clinical Cancer Research, 8:836-845 (2002)).

b. Rituximab-mediated Inhibition of the Raf-1/MEK1/2/ERK1/2 Pathway.

The above studies performed in the DLBCL 2F7 cell line following rituximab treatment did not address whether the same effects are observed in the FL-NHL cell lines. Studies in Ramos and Daudi revealed that rituximab selectively inhibited Bcl-_{XL} and leading to chemosensitization (Jazirehi, A. R. et al., Molecular Cancer Therapy, 2:1183-93 (2003)). We examined the underlying molecular mechanisms by which rituximab inhibits Bcl-_{xL} expression and demonstrate that rituximab inhibits the Raf-1/MEK1/2/ERK1/2 AP-1 signaling pathway and downregulates Bcl-_{xL} expression which is under the transcriptional regulation of AP-1. The ERK1/2 pathway is constitutively activated in Ramos and Daudi and its inhibition by rituximab sensitizes the cells to drug-induced apoptosis. The phosphorylation-dependent state of Raf-1/MEK1/2/ERK1/2 was significantly decreased 3-6 h post-rituximab treatment, concomitant with inhibition of MEK1/2 kinase ctivity. The role of the ERK1/2 pathway in the regulation of Bcl-2 and chemosensitization was corroborated by the use of specific pharmacologic inhibitors (GW-5074, PD-8098059, UO-126) which also sensitize the cells to drug-induced apoptosis. In addition, several lines of evidence corroborated the involvement of the ERK1/2 pathway and the regulation of Bcl-xL expression. Bcl-xL is abundantly expressed in lymphoma (Xerri, L. et al., British Journal of Hematology, 92:900-6 (1996)) and protects the cells from apoptosis induced by DNA-damaging agents. We have also analyzed mechanisms that underlie rituximab-mediated inhibition of the ERK1/2 pathway. Recently, Raf kinase inhibitor protein (RKIP) has been identified as a negative regulator of the ERK1/2 pathway (Yeung, K. et al., Nature, 401:173-7 (1999); Yeung, K. et al., Molecular Cellular Biology, 20:3079-85 (2000)). Therefore, we examined whether RKIP induction by rituximab was involved in the rituximab-induced inhibition of the ERK1/2 pathway. Our findings reveal that rituximab upregulates the expression of RKIP and facilitates the association of RKIP and Raf-1 (Jazirehi, A. R. et al., Cancer Research, 64:117-26 (2004)). These findings unravel a novel mechanism by which rituximab affects the ERK1/2 pathway and inhibits downstream selectively Bcl-_{xL} expression. This study identified several potential targets for therapeutic intervention (namely the components of the ERK1/2 pathway, Bcl-_{xL} and RKIP) and might provide a rational molecular basis for the therapeutic use of inhibitors of the ERK1/2 pathway in combination with chemotherapeutic drugs.

c. Rituximab-mediated Inhibition of the NFκB Pathway.

The demonstration that rituximab inhibits selectively Bcl-_{xL} expression in NHL cell lines suggested that rituximab may be inhibiting several signaling pathways that regulate Bcl-_{xL} expression. We have shown above that rituximab inhibits the ERK1/2 pathway leading to downregulation of Bcl-_{xL} expression. Previous findings reported that NFκB also regulates Bcl-_{xL} gene expression (Ghosh, S. et al., Cell, 109:81-96 (2002); Dixit, V. et al., Cell, 111:615-9 (2002)). Therefore, we examined whether rituximab modified the constitutive activation of the NFκB pathway in Ramos and Daudi cell lines. Indeed, we demonstrated that rituximab decreases the phosphorylation of NFκB-inducing kinase, IκB kinase, and IκB-α and diminishes IKK kinase activity and decreases NFκB DNA-binding activity. In addition, rituximab significantly upregulated RKIP expression, thus interrupting the NFκB signaling pathway concomitant with Bcl-_{xL} downregulation. The role of NFκB inhibition in downregulation of Bcl-_{xL} and chemosensitization was corroborated by the use of various inhibitors (Bay 11-7085, DHMEQ, SN-50). The induction of RKIP expression augmented its physical association with endogenous NIK, TAK-1 and IKK resulting in decreased activity of the NF-κB pathway and diminishing NF-κB DNA-binding activity and confirms the role of RKIP inhibition of NF-κB (Yeung, K. C. et al., Molecular Cellular Biology, 21:7207-17 (2001)). These studies revealed another novel signaling pathway triggered by rituximab and identified several potential targets for therapeutic intervention (that is the components of the NFκB pathway and RKIP). The findings also provide a rational molecular basis for the use of rituximab or NFκB pharmacologic inhibitors in combination with sub-toxic concentrations of chemotherapeutic drugs in rituximab and drug-refractory NHL.

d. Rituximab-induced Inhibition of the AKT Signaling Pathway.

As mentioned above, Bcl-_{xL} expression is transcriptionally regulated by various pathways and transcription factors. The AKT pathway was reported to regulate Bcl-_{xL} activity and expression (Vivanco, I. et al., Nature Reviews Cancer 2:489-501 (2002)). We have recently found that, indeed, rituximab treatment of Ramos cells inhibited the PI3K/AKT pathway, namely inhibition of phospho PI3K, PDK-1, AKT with no effect on non-phosphorylated proteins. In addition, inhibition of phospho-Bad by rituximab augmented the association of Bad with Bcl-_{xL} to form complexes. In addition, inhibition of the AKT pathway also inhibited the NFκB pathway and resulted in inhibition of Bcl-_{xL} expression as indicated above. The role of the AKT pathway in the regulation of chemoresistance was corroborated by the use of the AKT inhibitor Ly-294002 and by transfection with siRNA AKT (Suzuki, E. et al., Proc Amer Assoc Cancer Res volume 47 (2006)). As described above for the p38 MAPK, ERK1/2 and NF-κB pathways modified by rituximab, the present findings revealed another pathway inhibited by rituximab and identifies the AKT pathway as target for therapeutic intervention (see FIG. 14).

Chemosensitization of Drug-resistant B-NHL by Rituximab and Pharmacological Inhibitors

We have described above various signaling pathways inhibited by rituximab resulting in inhibition of apoptotic gene products and reversal of drug resistance. These studies indicated that the signaling pathways modified by rituximab are potential therapeutic targets and whose intervention can mimic rituximab-mediated chemosensitizing effects. Chemosensitization by rituximab and by pharmacological inhibitors that were studied to-date by us and were shown to reverse drug resistance in several NHL cell lines (namely 2F7, Raji Daudi and Ramos) are summarized in Table 1, farther below.

Rituximab-induced Immunosensitization of B-NHL

The above findings on cell-signaling by rituximab, namely inhibition of the survival signaling pathways (NFκB, ERK1/2, p38 MAPK and AKT), resulted in significant inhibition of anti-apoptotic gene products such as Bcl-2 and Bcl-_{xL}, and hence reversal of drug resistance. We have found that inhibition of the transcription repressor Yin Yang 1 (YY1) by S-nitrosylation (Hongo F. et al., Biochemistry and Biophysical Research Communications 336:692-701 (2005)) or by siRNA YY1 (Huerta-Yepez, S. et al., Oncogene 23:1993-5003 (2004)) resulted in upregulation of Fas expression and sensitization to FasL-induced apoptosis in ovarian and prostate carcinoma cell lines. We have shown that YY1 negatively regulates Fas transcription via its DNA-association to the silencer region of the Fas promoter. Three potential YY1 responsive elements were found to cluster in a very narrow sequence within the Fas promoter silencer region between −1619 and −1533 bp relative to the transcription initiation site. Deletion of the silencer region of the Fas promoter in a reporter assay resulted in augmentation of Fas expression of the tumor cells (Garban, H. J. et al., Journal of Immunology 167:75-81 (2001)). We have also reported that YY1 is downstream of NFκB and is regulated by NFκB activity. Since rituximab inhibited NFκB, we hypothesized that rituximab may also inhibit YY1 and sensitizes NHL cell lines to FasL-induced apoptosis. Indeed, our findings corroborated this hypothesis and demonstrated that treatment of FasL (or CH-11 agonist monoclonal antibody) resistant B-NHL cell lines with rituximab resulted in significant inhibition of YY1 expression and activity, upregulation of Fas expression and sensitization to CH-11-induced apoptosis. Fas expression was upregulated by rituximab treatment as early as 6 h as determined by flow cytometry for surface expression, reversed transcriptase polymerase chain reaction for transcription, and Western blot for total protein. Rituximab-induced inhibition of YY1 expression was determined by Western and its DNA activity by EMSA. The involvement of NFκB and YY1 in the regulation of Fas expression was corroborated by the use of Ramos with a dominant active inhibitor of NFκB and by silencing YY1 with YY1 siRNA, respectively. The role of rituximab-mediated inhibition of the p38 MAPK/NF-κB/YY1 pathways in the regulation of Fas and sensitization to CH-11-induced apoptosis was validated by the use of specific pharmacological inhibitors of these pathways, all of which resulted in sensitization to CH-11-induced apoptosis. The apoptotic pathway involved in rituximab-mediated sensitization of NHL cells to FasL-mediated apoptosis was examined. Treatment with rituximab alone did not have any effect on the activation of caspases or on the mitochondria. Likewise, treatment with CH-11 resulted in modest activation of caspases 8 and 9, which correlated with moderate induction of apoptosis. However, treatment with the combination resulted in mitochondrial depolarization, release of cytochrome-C and Smac/DIABLO, activation of caspases 9 and 3 and PARP cleavage, suggesting the involvement of the Type II mitochondrial apoptotic pathway (Barnhart, B. C. et al., Seminars in Immunology 15:185-93 (2003)). The activation of the mitochondrial pathway by combination of rituximab and CH-11 may be the result of the inhibition of the anti-apoptotic gene products Bcl-2/Bcl-_{xL} by rituximab.

Accordingly, rituximab appears to exert a new mechanism of action, namely the sensitization of tumor cells to host FasL-induced apoptosis.

Roles of BCLxl and YY1 in Chemoresistance and Immune-resistance in NHL, Respectively

Our findings clearly demonstrate that chemoresistance and Fas resistance in NHL cell lines are commonly regulated by the constitutive activation of NFκB. However, we demonstrate that chemoresistance and Fas resistance are differentially regulated by Bcl-_{xL} and YY1, respectively. Rituximab-mediated inhibition of NFκB activity resulted in both the inhibition of Bcl-_{xL} expression and chemosensitization and inhibition of YY 1 and sensitization to CH-11-induced apoptosis. These differentially regulated mechanisms for chemoresistance and immune-resistance emanated from findings making use of biologically engineered cell lines and specific pharmacological inhibitors. Treatment with specific inhibitors for NFκB sensitized NHL cells to both drug and CH-11-induced apoptosis. The role of Bcl-_{xL} expression in the regulation of drug resistance, but not Fas resistance, was demonstrated by the failure of rituximab to sensitize Bcl-_{xL} overexpressing Ramos cells to drug-induced apoptosis, although the same cells were still sensitive to rituximab-induced sensitization to CH-11 apoptosis.

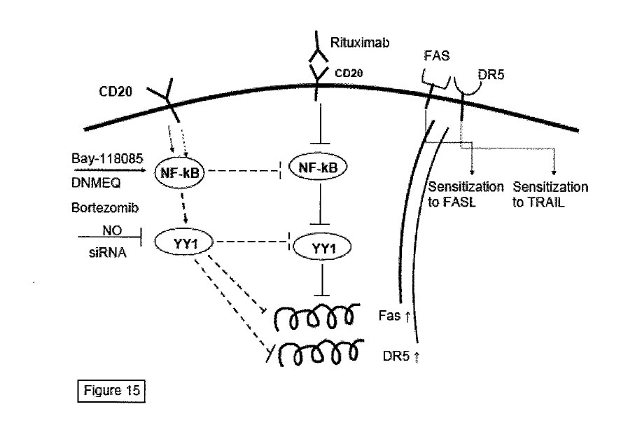

These findings clearly establish distinct regulatory mechanisms modulated by rituximab in NHL cells downstream of NFκB for the sensitization to Fas and drug-induced apoptosis. This finding have clear clinical implications and suggest that overexpression of Bcl-_{xL} in tumors, which are refractory to treatment with chemotherapeutic drugs, alone or in combination with rituximab, may still be sensitive to killing by rituximab in combination with immunotherapy (Vega, M. I. et al., Journal of Immunology 175:2174-83 (2005)) (see FIG. 15). Accordingly, the invention in one aspect provides methods of treating patients who are ritixumab resistant by administering another agent which modulates one or more components of a rituximab signal pathway in a direction similar to that of rituximab to promote cancer cell death.

Failure of Rituximab to Signal Rituximab-resistant NHL Clones

While rituximab used as monotherapy or in combination with chemotherapy has improved significantly the treatment of patients with NHL, there remains the problem of patients initially unresponsive to rituximab and a subset of patients experiencing unresponsiveness to further treatment. The mechanisms of unresponsiveness have not been clear. It has been postulated that CD20 downregulation (Kennedy, A. D. et al., Journal of Immunology 172:3280-8 (2004)), loss of CD20 (Haidar, J. H. et al., European Journal of Hematology 70:330-2 (2003)), and circulating CD20 (Manshouri, T. et al., Blood 101:2507-13 (2003)) may be responsible for resistance.

Based on our findings above with rituximab-mediated inhibition of cell signaling, we find that the development of rituximab resistance emanates from failure of rituximab to signal the cells effectively, as well as the development of hyper-activated survival signaling pathways and upregulation of anti-apoptotic gene products. In order to test our hypothesis, we have generated in vitro rituximab-resistant (RR) clones from Ramos, Daudi, and 2F7 cells by culturing the cells in the presence of increasing concentrations of rituximab for several weeks and multiple cycles of limited dilutions.

We have examined representative clones (2F7RR1, Ramos RR1, and Daudi RR1) for their response to rituximab as compared to wildtype (wt) cells. Preliminary findings have been presented (Jazirehi, A. R. et al., Blood 104:3410 (Abstract) (2004); Jazirehi, A. R. et al., Blood 106:1514 (Abstract) (2005); Vega, M. et al., Proc Amer Assoc Cancer Res 47. (2006)). Examination of these clones revealed that they express some loss of CD20 on the cell surface, were not responsive to complement-dependent cytotoxicity, antibody-dependent cellular cytotoxicity, were not growth-inhibited by rituximab, nor underwent apoptosis following cross-linking of rituximab with a secondary anti-human IgG. We then examined the cell-signaling mediated by rituximab on resistant clones compared to wt. Preliminary findings demonstrate that in wt Daudi cells, rituximab induces a rapid and transient increase in A-SMase activity paralleled with cellular ceramide generation in lipid rafts. In addition, rituximab treatment externalizes both ceramide and A-SMase which co-localizes with the CD20 receptor (Bezombes, C. et al., Blood, 104:1166-73 (2004)). In the resistant clones, however, rituximab-induced A-SMase translocation and ceramide generation at the cell surface was reduced (Jazirehi, A. R. et al., Proc Amer Assoc Cancer Research 47 (2006)).

These findings indicate that the failure of rituximab to mediate cell signaling may be due to failure of CD20 migration to lipid rafts and initiating cell signaling. Further studies revealed that rituximab failed to inhibit the p38 MAPK, ERK1/2 and NF-κB signaling pathways. In addition, we have found that the clones show hyper-activation of these pathways with overexpression of Bcl-2/Bcl-_{xL}. Noteworthy, the clones showed cross-resistance to high concentrations of chemotherapeutic drug-induced apoptosis compared to the response seen in wt. We then examined if the RR clones' failure to be chemosensitized by rituximab may be reverted to sensitivity. Our previous finding with wt cells revealed that sensitizing agents that interfere with the above signaling pathways modified by rituximab resulted in significant chemosensitization that were comparable to rituximab. Thus, we examined if such sensitizing agents can reverse the resistance in the RR clones. Indeed, our findings established that treatment of RR clones with inhibitors of the NF-κB pathway (e.g. DHMEQ, Bortezomib, Bay 11-7085), the ERK1/2 pathway (e.g. PD-098059), the p38 MAPK pathway (e.g. SB-203580) and Bcl-2 (e.g. 2MAM-A3) sensitized the resistant clones to various chemotherapeutic drugs (e.g. Paclitaxel, Vincristine, VP-16, CDDP, and Adriamycin).

These findings indicate that the acquired or development of resistance of NHL cells to rituximab, alone or in combination with chemotherapy, may still be amenable to treatment by combination of sensitizing agents described above in combination with low doses of conventional chemotherapy. In addition, these findings present several new potential targets for the generation of new class of inhibitors to reverse resistance. (See FIG. 14). Accordingly, treatment with rituximab and immuno-modulating agents that upregulate the expression of death ligands on host effector cells, or co-administration of rituximab and TRAIL/anti-DR4/DR5, mAb can be useful in treating cancer.

List of Abbreviations

ADCC: antibody-dependent cell-mediated cytotoxicity
AP-1: activator protein-1
ARL: acquired immunodeficiency syndrome (AIDS)-related lymphoma
Bcl-2: B cell lymphoma protein 2
Bcl-_{XL}: Bcl-2 related gene (long alternatively spliced variant of Bcl-x gene)
CDC: complement-dependent cytotoxicity
DBA: DNA-binding activity
DHMEQ: dehydroxymethylepoxyquinomicin
DLBCL: diffuse large B-cell lymphoma
ERK1/2 MAPK: extracellular signal-regulated kinase 1/2 mitogen activated protein kinase
IKK: inhibitor of kappa B (I□B) kinase complex
FACS: fluorescence activated cell sorter
Mcl-1: myeloid cell differentiation 1
MDR: multi-drug resistance
MEK1/2: mitogen activated protein kinase kinase 1/2
2MAM-A3: 2-methoxyantimycin-A3
NIK: nuclear factor κB (NF-κB) inducing kinase
PD098059: [2-(2′-amino-3′-methoxyphenyl)-oxanaphthalen-4-one]
RIPA: radioimmuno-precipitation assay
RKIP: Raf-1 kinase inhibitor protein
TRAIL: tumor necrosis factor (TNF)-related apoptosis-inducing ligand
XTT: sodium 3-[1-(phenylamino-carbonyl)-3, 4 tetrazolium]-bis (4-metoxy-6-nitro) benzene Sulfonic acid hydrate
   Definitions

Markers for use according to the invention include gene products that are modified by a rituximab triggered signaling pathway disclosed herein. These markers include Bcl-2 family members, surviving, IAPs, and cytokines. Exemplary markers include “Functional or activated Bcl-2/Bcl-_{XL}, AKT, PTEN, Fas, YY1, NFκB, NIK, IKK, IKB, and transcription factors AP-1 and STAT3.” The recitals of markers refers to nucleic acids, e.g., gene, pre-mRNA, mRNA, and functional or activated polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide or a member of rituximab signaling pathway disclosed herein, or modulated by a ritiuximab signaling pathway disclosed herein or encoded by a referenced nucleic acid or an amino acid sequence described herein; they include the human and wt marker proteins (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. The genes and protein sequences for functional or activated Bcl-2/Bcl-_{XL}, AKT, PTEN, Fas, YY1, NFκB, NIK, IKK, IKB, and transcription factors AP-1 and STAT3 and other members of the rituximab triggered signaling pathways disclosed herein are well known in the art. Truncated and alternatively spliced forms are included in the definition. In addition, the definition includes phosphorylated forms and enzymatically active or functional forms. Exemplary accession numbers include the following: NP_{—}644805, NP_{—}001014432, NP_{—}000305, AAB35516, NP_{—}003394, NP_{—}003989, BAA33714, AAD08996, AAD13528; AAC64675, and NP_{—}0065390.

The phrase “molecular signaling pathways triggered by rituximab in cancer cells, including functional or activated A Bcl-2/Bcl-_{XL}, AKT, PTEN, Fas, YY1, NFκB, NIK, IKK, IKB, and transcription factors AP-1 and STAT3 refers to pathways disclosed herein, including NFκB and AKT, and polypeptide members thereof known in the art.

“Functional or activated” refers to the altered activation state of molecular pathways triggered by rituximab. The studies described herein show that rituximab alters the activation state of the survival signaling pathways (e.g., ERK1/2; NFkB, p38MAPK, AKT). The activation state is determined by phosphorylation and enzymatic activity. The basal non phosphorylated protein levels are not altered by rituximab. In the resistant clones these activated signaling pathways are not inhibited by rituximab and particularly are hyperactivated.

“Cancer” refers to human cancers and carcinomas, sarcomas, adenocarcinomas, lymphomas, leukemias, chronic lymphocytic leukemia, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CML), and multiple myeloma. The invention is useful for any cancer or cell that expresses CD-20 such as lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CML), chronic lymphocytic leukemia, and multiple myeloma, as well as for B cell mediated diseases such as autoimmune diseases, rheumatoid arthritis, lupus, transplantation, etc.

“Therapy resistant” or “chemo-resistant” cancers, tumor cells, and tumors refers to cancers that have become resistant to both apoptosis-mediated (e.g., through death receptor cell signaling, for example, Fas ligand receptor, TRAIL receptors, TNF-R1) and non-apoptosis mediated (e.g., antimetabolites, anti-angiogenic, etc.) cancer therapies. “Therapy sensitive” cancers are not resistant to therapy. Cancer therapies include chemotherapy, hormonal therapy, radiotherapy, gene therapy, and immunotherapy (e.g., vaccines). Such therapies include administration of chemotherapeutic drugs, antibodies, immunotoxins, proteasome inhibitors, or chemical inhibitors. The agents can mediate both apoptosis and non apoptosis mediated cytotoxicity.

In a preferred embodiment, the methods of the invention can be used to evaluate or diagnose whether a cancer is resistant with respect to a variety of anticancer agents that induce or stimulate apoptosis and inform the selection of a therapy avoiding or opposing the mechanism of resistance. These include, but are not limited to, radiation (e.g., X-rays, gamma rays, UV); tumor necrosis factor (TNF)-related factors (e.g., TNF family receptor proteins, TNF family ligands, TRAIL, antibodies to TRAILR1 or TRAILR2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the proapoptitic agents include anti-hyperproliferative or antineoplastic agents selected from alkylating agents, and antimetabolites.

Alkylating agents can be 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, the antimetabolites can be 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, the pro-apoptosis and/or chemotherapeutic agents can be 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Resistance to any oncolytic agent that is routinely used in a cancer therapy may be evaluated by assessing the state of the rituximab-triggered pathways and their components. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents. Listed agents include aldesleukin, Alemtuzumab, allopurinol, arsenic trioxide, asparaginase azacitidine, bevacizumab, bexarotene, bortezomib, busulfan, capecitabine, carboplatin, carboplatin, carmustine, cetuximab, chlorambucil, cisplatin, cladribine, clofarabine, cyclophosphamide, cytarabine, dacarbazine, dactinomycin, actinomycin D, dasatinib, daunomycin, decitabine, denileukin, diftitox, , docetaxel, doxorubicin, erlotinib, etoposide phosphate, floxuridine, fludarabine, 5-fluorouracil, fulvestrant, gefitinib, gemcitabine, gemtuzumab ozogamicin, goserelin acetate, goserelin, histrelin acetate, Ibritumomab Tiuxetan, idarubicin ifosfamide, imatinib mesylate Interferon alfa-2a, Interferon alfa-2b, irinotecan, lenalidomide, letrozole, leucovorin, Leuprolide, levamisole, lomustine, CCNU, meclorethamine, nitrogen mustard, megestrol acetate, melphalan, L-PAM, 6-mercaptopurine, methotrexate, methoxsalen, mitomycin C, mitotane, mitoxantrone, nandrolone phenpropionate, nelarabine, nofetumomab, oxaliplatin, paclitaxel, pamidronate, pegaspargase, pentostatin, pipobroman, plicamycin, mithramycin, procarbazine, quinacrine, Rituximab, sorafenib, streptozocin, sunitinib maleate, tamoxifen, temozolomide, teniposide, VM-26, testolactone, thalidomide, 6-thioguanine, thiotepa, topotecan, toremifene, Tositumomab, Trastuzumab, tretinoin, ATRA, Uracil Mustard, valrubicin, vinblastine, vincristine, vinorelbine.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

“Therapy-mediated or induced cytotoxicity” refers to all mechanisms by which cancer therapies kill or inhibit cancer cells, including but not limited to inhibition of proliferation, inhibition of angiogenesis, and cell death due to, for example, activation of apoptosis pathways (e.g., death receptor cell signaling, for example, Fas ligand receptor, TRAIL receptors, TNF-R1). Cancer therapies include chemotherapy, immunotherapy, radiotherapy, and hormonal therapy.

“Therapeutic treatment” and “cancer therapies” and “cancer therapy reagents” refers to apoptosis-mediated and non-apoptosis mediated cancer therapies that treat, prevent, or inhibit cancers, including chemotherapy, hormonal therapy (e.g., androgens, estrogens, antiestrogens (tamoxifen), progestins, thyroid hormones and adrenal cortical compounds), radiotherapy, and immunotherapy (e.g., ZEVALIN, BEXXAR, RITUXAN (rituximab), HERCEPTIN). Cancer therapies can be enhanced by administration with a sensitizing agent, as described herein, either before or with the cancer therapy.

“Chemotherapeutic drugs” include conventional chemotherapeutic reagents such as alkylating agents, anti-metabolites, anti-mitototics, plant alkaloids, antibiotics, and miscellaneous compounds e.g., cis-platinum, CDDP, methotrexate, vincristine, adriamycin, bleomycin, and hydroxyurea. Chemotherapeutic drugs also include proteasome inhibitors such as salinosporamides, bortezomib, PS-519, and omuralide. The drugs can be administered alone or combination (“combination chemotherapy”).

By “sensitizingly effective amount or dose” or “sensitizingly sufficient amount or dose” herein is meant a dose or a modulator or agent that produces cancer cell sensitizing effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Sensitized cancer cells respond better to cancer therapy (are inhibited or killed faster or more often) than non-sensitized cells, as follows: Control samples (untreated with sensitizing agents) are assigned a relative cancer therapy response value of 100%. Sensitization is achieved when the cancer therapy response value relative to the control is about 110% or 120%, preferably 200%, more preferably 500-1000% or more, i.e., at least about 10% more cells are killed or inhibited, or the cells are killed or inhibited at least about 10% faster. Cancer therapy response value refers to the amount of killing or inhibition of a cancer cell, or the speed of killing or inhibition of a cancer cell when it is treated with a cancer therapy. Some compounds are useful both as therapeutic reagents and as sensitizing reagents. Often, a lower dose (i.e., lower than the conventional therapeutic dose) or sub-toxic dose of such a reagent can be used to sensitize a cell. Often, when a cell is sensitized, a lower dose of the chemotherapeutic reagent can be used to achieve the same therapeutic effect as with a cell that has not been sensitized.

By “therapeutically effective amount or dose” or “therapeutically sufficient amount or dose” herein is meant a dose or a modulator or agent that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

The terms “overexpress,” “overexpression” or “overexpressed” interchangeably refer to a gene that is transcribed or translated at a detectably greater level, usually in a cancer cell, in comparison to a normal cell. Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as a transcription factor, as a DNA binding factor. Overexpression can be detected using conventional techniques for detecting mRNA (i.e., RT-PCR, PCR, hybridization) or proteins (i.e., ELISA, Western blots, flow cytometry, immunofluorescence, immunohistochemical, DNA binding assay techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

The terms “underexpress,” “underexpression” or “underexpressed” interchangeably refer to a gene that is transcribed or translated at a detectably lower level, usually in a cancer cell, in comparison to a normal cell. Underexpression therefore refers to both underexpression of protein and RNA (due to decreased transcription, post transcriptional processing, translation, post translational processing, altered stability, and altered protein degradation), as well as local underexpression due to altered protein traffic patterns (decreased nuclear localization), and altered functional activity, e.g., as an enzyme. Underexpression can be detected using conventional techniques for detecting mRNA (i.e., RT-PCR, PCR, hybridization) or proteins (i.e., ELISA, Western blots, flow cytometry, immunofluorescence, immunohistochemical, DNA binding assay techniques). Underexpression can be 90%, 80%, 70%, 60%, 50%, 40%, 30%, 20%, 10% or less in comparison to a normal cell. In certain instances, underexpression is 1-fold, 2-fold, 3-fold, 4-fold or more lower levels of transcription or translation in comparison to a normal cell.

The terms “cancer-associated antigen” or “tumor-specific marker” or “tumor marker” interchangeably refers to a molecule (typically protein, carbohydrate or lipid) that is preferentially expressed or over expressed in a cancer cell (e.g., on the cell surface or intracellularly) in comparison to a normal cell, and which is useful for the preferential targeting of a pharmacological agent to the cancer cell. A marker or antigen can be expressed on the cell surface or intracellularly. Oftentimes, a cancer-associated antigen is a molecule that is overexpressed, underexpressed in a cancer cell in comparison to a normal cell. Oftentimes, a cancer-associated antigen is a molecule that is inappropriately synthesized in the cancer cell, for instance, a molecule that contains deletions, additions or mutations in comparison to the molecule expressed on a normal cell. A cancer associated antigen can have minimal degradation in the cancer cell. Oftentimes, a cancer-associated antigen will be expressed exclusively in a cancer cell and not synthesized or expressed in a normal cell. Exemplified cell surface tumor markers include the proteins c-erbB-2, and PSMA for prostate cancer. Exemplified intracellular tumor markers include, for example, mutated tumor suppressor or cell cycle proteins, including p53.

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention. An agonist may inhibit or activate signaling pathways according to its action.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. They may act directly or indirectly. The term “modulator” includes inhibitors and activators. Inhibitors are agents that, e.g., inhibit expression, e.g., translation, post-translational processing, stability, degradation, or nuclear or cytoplasmic localization of a polypeptide, or transcription, post transcriptional processing, stability or degradation of a polynucleotide of the invention or bind to, partially or totally block stimulation, DNA binding, transcription factor activity or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation, DNA binding or enzymatic activity, sensitize or up regulate the activity of a polypeptide or polynucleotide of the invention, e.g., agonists. Modulators include naturally occurring and synthetic ligands, antagonists, agonists, small chemical molecules, antibodies, inhibitory RNA molecules (i.e., siRNA or antisense RNA) and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1 %. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

The term “nitric oxide donor” or “NO donor” refers to any compound capable of the intracellular delivery of nitric oxide. Typically, an NO donor is any compound capable of denitrition that releases nitric oxide. Also included are those compounds that can be metabolized in vivo into a compound which delivers nitric oxide (e.g., a prodrug form of a NO donor). An NO donor can be a synthetic or naturally occurring organic chemical compound and can be a polypeptide. Exemplified pharmaceutical agents that are NO donors include arginine (L- and D-), amyl nitrite, isoamyl nitrite, nitroglycerin, isosorbide dinitrate, isosorbide-5-mononitrate, erythrityl tetranitrate. Nitric oxide synthases, both constitutive and inducible forms, are also nitric oxide donors.

The term “inducer of inducible nitric oxide synthase (iNOS)” or “activator of iNOS” refers to any compound that promotes the expression (transcription or translation) and/or promotes that catalytic activity of iNOS.

A “cell-cycle-specific” or “antimitotic” or “cytoskeletal-interacting” drug interchangeably refer to any pharmacological agent that blocks cells in mitosis. Generally, cell-cycle-specific-drugs bind to the cytoskeletal protein tubulin and block the ability of tubulin to polymerize into microtubules, resulting in the arrest of cell division at metaphase. Exemplified cell-cycle-specific drugs include vinca alkaloids, taxanes, colchicine, and podophyllotoxin. Exemplified vinca alkaloids include vinblastine, vincristine, vindesine and vinorelbine. Exemplifed taxanes include paclitaxel and docetaxel. Another example of a cytoskeletal-interacting drug includes 2-methoxyestradiol.

Rituximab refers to a chimeric murine/human monoclonal antibody which targets the CD20 antigen which can be found on the surface of normal and malignant B lymphocytes. Rituximab itself is an IgG₁ kappa immunoglobulin containing murine light- and heavy-chain variable region sequences and human constant region sequences. Rituximab is reported to have two heavy chains of 451 amino acids and two light chains of 213 amino acids (based on cDNA analysis) with an approximate molecular weight of 145 kD. The binding affinity of rituximab for the CD20 antigen of approximately 8.0 nM. While rituximab is referenced throughout the specification other antibodies which target the CD20 antigen and trigger signaling mediated by the CD20 antigen are also suitable.

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

“Determining the functional effect” refers to assaying for a compound that increases or decreases a parameter that is indirectly or directly under the influence of a polynucleotide or polypeptide of the invention, e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies, binding to DNA; measuring changes in ligand binding affinity; measurement of calcium influx; measurement of the accumulation of an enzymatic product of a polypeptide of the invention or depletion of an substrate; changes in enzymatic activity, e.g., kinase activity, measurement of changes in protein levels of a polypeptide of the invention; measurement of RNA stability; G-protein binding; GPCR phosphorylation or dephosphorylation; signal transduction, e.g., receptor-ligand interactions, second messenger concentrations (e.g., cAMP, IP3, or intracellular Ca2+); identification of downstream or reporter gene expression (CAT, luciferase, β-gal, GFP and the like), e.g., via chemiluminescence, fluorescence, colorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

“Biological sample” or “tissue sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell), the size and type of the tumor (i.e., solid or suspended (i.e., blood or ascites)), among other factors. Representative biopsy techniques include excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison 's Principles of Internal Medicine, Kasper, et al., eds., 16th ed., 2005, Chapter 70, and throughout Part V.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res., 25:3389-3402 (1977) and Altschul et al., J. Mol. Biol., 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of functional or activated Bcl-2/Bcl-_{XL}, AKT, PTEN, Fas, YY1, NFκB, NIK, IKK, IKB, and transcription factors AP-1 and STAT3. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created. Truncated forms of YY1 are described, for example, in Begon et al., J Biol Chem, 280:24428 (2005); Krippner-Heidenreich, et al., Mol Cell Biol, 25:3704 (2005); Nishiyama et al., Biosci Biotechnol Biochem, 67:654 (2003); and Berndt et al., J Neurochem, 77:935 (2001).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²p, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications, Academic Press, Inc. N.Y. (1990)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature, 348:552-554 (1990)).

For preparation of antibodies, e.g., recombinant, monoclonal, or polyclonal antibodies, many technique known in the art can be used (see, e.g., Kohler & Milstein, Nature, 256:495-497 (1975); Kozbor et al., Immunology Today, 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); and Lonberg & Huszar, Intern. Rev. Immunol., 13:65-93 (1995)). Alternatively, phage display technology can be used to identify antibodies and heteromeric Fab fragments that specifically bind to selected antigens (see, e.g., McCafferty et al., Nature, 348:552-554 (1990); Marks et al., Biotechnology, 10:779-783 (1992)). Antibodies can also be made bispecific, i.e., able to recognize two different antigens (see, e.g., WO 93/08829, Traunecker et al., EMBO J., 10:3655-3659 (1991); and Suresh et al., Methods in Enzymology, 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature, 321:522-525 (1986); Riechmann et al., Nature, 332:323-327 (1988); Verhoeyen et al., Science, 239:1534-1536 (1988) and Presta, Curr. Op. Struct. Biol., 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immumunoassay formats and conditions that can be used to determine specific immunoreactivity).

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science, 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

Assays for Modulators of Cell Gene Products

Modulation of functional or activated gene products (e.g., Bcl-2/Bcl-_{XL}, AKT, PTEN, Fas, YY1, NFκB, NIK, IKK, IKB, and transcription factors AP-1 and STAT3), and corresponding modulation of cellular, e.g., tumor cell, proliferation, can be assessed using a variety of in vitro and in vivo assays, including cell-based models. Such assays can be used to test for cell gene products or inhibitors and activators of functional or activated Bcl-2/Bcl-_{XL}, AKT, PTEN, Fas, YY1, NFκB, NIK, IKK, IKB, and transcription factors AP-1 and STAT3 transcription or translation, or protein activity, and consequently, inhibitors and activators of cellular proliferation, including modulators of chemotherapeutic and immunotherapeutic sensitivity and toxicity. Assays for modulation include cell-viability, cell proliferation, cell responses to apoptotic stimuli, gene transcription, mRNA arrays, kinase or phosphatase activity, interaction with other proteins including other transcription factors, and DNA binding, gene transfection assays, siRNA, Western assays, reporters assays, and intracellular localization by confocal microscopy. Such modulators are useful for treating disorders related to pathological cell proliferation, e.g., cancer, autoimmunity, aging. Modulators can be tested using in vivo well cells expressing functional or activated Bcl-2/Bcl-_{XL}, AKT, PTEN, Fas, YY 1, NFκB, NIK, IKK, IKB, and transcription factors AP-1 and STAT3 and in vitro well, either recombinant or naturally occurring YY1 protein, preferably human YY1. Wild type as well as truncated and alternatively spliced forms of functional or activated Bcl-2/Bcl-_{XL}, AKT, PTEN, Fas, YY1, NFκB, NIK, IKK, IKB, and transcription factors AP-1 and STAT3 are useful targets.

Measurement of cellular proliferation by modulation with a protein or a nucleic acid, either recombinant or naturally occurring, can be performed using a variety of assays, in vitro, in vivo, and ex vivo, as described herein. A suitable physical, chemical or phenotypic change that affects activity, e.g., enzymatic activity such as kinase activity, cell proliferation, or ligand binding (e.g., a protein or nucleic acid receptor) can be used to assess the influence of a test compound on the polypeptide of this invention. When the functional effects are determined using intact cells or animals, one can also measure a variety of effects, such as, ligand binding, DNA binding, kinase activity, transcriptional changes to both known and uncharacterized genetic markers (e.g., Northern blots), changes in cell metabolism, changes related to cellular proliferation, cell surface marker expression, DNA synthesis, marker and dye dilution assays (e.g., GFP and cell tracker assays), contact inhibition, tumor growth in nude mice, reporter assays, overexpression or knockout gene products, and siRNA, etc.

In Vitro Assays

Assays to identify modulator sor compounds with modulating activity can be performed in vitro. Such assays can use a full length protein or a variant thereof, or a mutant thereof, a truncated form or a fragment of a protein. Purified recombinant or naturally occurring protein can be used in the in vitro methods of the invention. In addition to purified protein, the recombinant or naturally occurring protein can be part of a cellular lysate or a cell membrane. As described below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein. Other in vitro assays include enzymatic activity assays, such as phosphorylation or autophosphorylation assays). Preferred in vitro assay systems include DNA binding assays (EMSA). In addition, cell based reporter assays, whole cell treatment assays with checking for a gene product by Western, PCR, and microarray methods for example can be used. Such products can be analyzed by immunohistochemistry, immunofluorescence, confocal and microscopy, for example.

In one embodiment, a high throughput binding assay is performed in which the protein, a truncated form or a fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described herein, including small organic molecules, peptides, antibodies, and binding protein or nucleic acid analogs. A wide variety of assays can be used to identify modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator.

In one embodiment, microtiter plates are first coated with either a protein or a binding protein (ie. antibody, transcription factors, etc.) or nucleic acid, and then exposed to one or more test compounds potentially capable of inhibiting the binding of a protein to a binding protein or nucleic acid. A labeled (i.e., fluorescent, enzymatic, radioactive isotope) binding partner of the coated protein, either a binding protein or nucleic acid, or a protein, is then exposed to the coated protein and test compounds. Unbound protein (or nucleic acid) is washed away as necessary in between exposures to a protein, a binding protein or nucleic acid, or a test compound. An absence of detectable signal indicates that the test compound inhibited the binding interaction between a protein and a binding protein or nucleic acid. The presence of detectable signal (i.e., fluorescence, calorimetric, radioactivity) indicates that the test compound did not inhibit the binding interaction between a protein and a binding protein or nucleic acid. One can also use chromatographic techniques, for example HPLC, and evaluate elution profiles of protein alone and protein complexed with other factors, including DNA and/or other transcription factors. The presence or absence of detectable signal is compared to a control sample that was not exposed to a test compound, which exhibits uninhibited signal. In some embodiments the binding partner is unlabeled, but exposed to a labeled antibody that specifically binds the binding partner.

Cell-based in Vivo Assays

In another embodiment, protein is expressed in a cell, and functional, e.g., physical and chemical or phenotypic, changes are assayed to identify modulators of cellular proliferation, e.g., tumor cell proliferation. In some embodiments, as exemplified in some instances herein, the cells can be chemo- or treatment-resistant cell lines or clones, including clones of lymphoma, or leukemia cells. Cells expressing recombinant or endogenous proteins can also be used in binding assays and enzymatic assays. Preferably, the cells overexpress or under express protein in comparison to a normal cell of the same type. Any suitable functional effect can be measured, as described herein. For example, cellular morphology (e.g., cell volume, nuclear volume, cell perimeter, and nuclear perimeter), ligand binding, kinase activity, apoptosis, cell surface marker expression, cellular proliferation, cellular localization of proteins or transcripts, DNA binding, GFP positivity and dye dilution assays (e.g., cell tracker assays with dyes that bind to cell membranes), DNA synthesis assays (e.g., ³H-thymidine and fluorescent DNA-binding dyes such as BrdU or Hoechst dye with FACS analysis), are all suitable assays to identify potential modulators using a cell based system. Suitable cells for such cell based assays include both primary cancer or tumor cells and cell lines, as described herein, e.g., A549 (lung), MCF7 (breast, p53 wild-type), H1299 (lung, p53 null), Hela (cervical), PC3 (prostate, p53 mutant), MDA-MB-231 (breast, p53 wild-type). Variants derived from these cell lines with specific gene modification will also be used. Cancer cell lines can be p53 mutant, p53 null, or express wild type p53. The protein can be naturally occurring or recombinant. Also, truncated forms or fragments or chimeric proteins can be used in cell based assays.

Cellular polypeptide levels can be determined by measuring the level of protein or mRNA. The level of protein or related proteins are measured using immunoassays such as western blotting, ELISA, immunofluorescence and the like with an antibody that selectively binds to the polypeptide or a fragment thereof. For measurement of mRNA, amplification, e.g., using PCR, RT-PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein. It is also useful to observe protein translocation into the nucleus and other cellular compartments by, for example, confocal microscopy.

Alternatively, protein expression can be measured using a reporter gene system. Such a system can be devised using a protein promoter which modulates transcription of the protein, or a protein responsive site, which is modulated by binding of the protein, operably linked to a reporter gene, including chloramphenicol acetyltransferase, firefly luciferase, bacterial luciferase, β-galactosidase, green fluorescent protein (GFP) and alkaline phosphatase. Furthermore, the protein of interest can be used as an indirect reporter via attachment to a second reporter such as red or green fluorescent protein (see, e.g., Mistili & Spector, Nature Biotechnology, 15:961-964 (1997)). The reporter construct is typically transfected into a cell. After treatment with a potential modulator, the amount of reporter gene transcription, translation, or activity is measured according to standard techniques known to those of skill in the art. In a preferred embodiment, plasmids that allow for stable transfection are used.

Animal Models

Animal models of cellular proliferation also find use in screening for modulators of cellular proliferation. Similarly, transgenic animal technology including gene knockout technology, for example, as a result of homologous recombination with an appropriate gene targeting vector, or gene overexpression, will result in the absence or increased expression of the protein. The same technology can also be applied to make knock-out cells. When desired, tissue-specific expression or knockout of the protein may be necessary. Transgenic animals generated by such methods find use as animal models of cellular proliferation and are additionally useful in screening for modulators of cellular proliferation.

Knock-out cells and transgenic mice can be made by insertion of a marker gene or other heterologous gene into an endogenous gene site in the mouse genome via homologous recombination. Such mice can also be made by substituting an endogenous gene with a mutated version of the gene, or by mutating an endogenous gene, e.g., by exposure to carcinogens.

In particular, human tumor xenografts can be used and the tissues be examined for the effects of a treatment on gene modifications by IHC, ISH, RT-PCR, Western, etc. Treatments can also be with various sensitizing agents in combination with drugs.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science, 244:1288 (1989)). Chimeric targeted mice can be derived according to Hogan et al., Manipulating the Mouse Embryo: A Laboratory Manual, Cold Spring Harbor Laboratory (1988), Teratocarcinomas and Embryonic Stem Cells: A Practical Approach, Robertson, ed., IRL Press, Washington, D.C., (1987), and Pinkert, Transgenic Animal Technology: A Laboratory Handbook, Academic Press (2003).

Exemplary Assays

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Soft agar growth or colony formation in suspension assays can be used to identify modulators. Typically, transformed host cells (e.g., cells that grow on soft agar) are used in this assay. For example, RKO or HCT116 cell lines can be used. Techniques for soft agar growth or colony formation in suspension assays are described in Freshney, Culture of Animal Cells a Manual of Basic Technique, 3^{rd} ed., Wiley-Liss, New York (1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with [³H]-thymidine at saturation density can be used to measure density limitation of growth. See Freshney (1994), supra. The transformed cells, when contacted with cellular proliferation modulators, regenerate a normal phenotype and become contact inhibited and would grow to a lower density.

Contact inhibition and density limitation of growth assays can be used to identify modulators which are capable of inhibiting abnormal proliferation and transformation in host cells. Typically, transformed host cells (e.g., cells that are not contact inhibited) are used in this assay. For example, RKO or HCT116 cell lines can be used. In this assay, labeling index with [³H]-thymidine at saturation density is a preferred method of measuring density limitation of growth. Transformed host cells are contacted with a potential modulator and are grown for 24 hours at saturation density in non-limiting medium conditions. The percentage of cells labeling with [³H]-thymidine is determined autoradiographically. See, Freshney (1994), supra. The host cells contacted with a modulator would give arise to a lower labeling index compared to control (e.g., transformed host cells transfected with a vector lacking an insert).

Growth Factor or Serum Dependence

Growth factor or serum dependence can be used as an assay to identify modulators. Transformed cells have a lower serum dependence than their normal counterparts (see, e.g., Temin, J. Natl. Cancer Insti., 37:167-175 (1966); Eagle et al., J. Exp. Med., 131:836-879 (1970)); Freshney, supra. This is in part due to release of various growth factors by the transformed cells. When transformed cells are contacted with a modulator, the cells would reacquire serum dependence and would release growth factors at a lower level.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. In Mihich (ed.): “Biological Responses in Cancer.” New York, Academic Press, pp. 178-184 (1985)). Similarly, tumor angiogenesis factor (TAF) is released at a higher level in tumor cells than their normal counterparts. See, e.g., Folkman, Angiogenesis and cancer, Sem Cancer Biol. (1992)). Other exemplified tumor specific markers include growth factors and cytokines.

Tumor specific markers can be assayed to identify modulators which decrease the level of release of these markers from host cells. Typically, transformed or tumorigenic host cells are used. Various techniques which measure the release of these factors are described in Freshney (1994), supra. Also, see, Unkless et al., J. Biol. Chem., 249:4295-4305 (1974); Strickland & Beers, J. Biol. Chem., 251:5694-5702 (1976); Whur et al., Br. J. Cancer, 42:305-312 (1980); Gulino, Angiogenesis, tumor vascularization, and potential interference with tumor growth. In Mihich, E. (ed): “Biological Responses in Cancer.” New York, Plenum (1985); Freshney, Anticancer Res., 5:111-130 (1985).

Invasiveness into Matrigel

The degree of invasiveness into Matrigel or some other extracellular matrix constituent can be used as an assay to identify modulators which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used as host cells. Therefore, modulators can be identified by measuring changes in the level of invasiveness between the host cells before and after the introduction of potential modulators. If a compound modulates a protein, its expression in tumorigenic host cells would affect invasiveness.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

G₀/G₁ Cell Cycle Arrest Analysis

G₀/G₁ cell cycle arrest can be used as an assay to identify modulators. In this assay, cell lines, such as RKO or HCT116, can be used to screen modulators. The cells can be co-transfected with a construct comprising a marker gene, such as a gene that encodes green fluorescent protein, or a cell tracker dye. Methods known in the art can be used to measure the degree of G₁ cell cycle arrest. For example, a propidium iodide signal can be used as a measure for DNA content to determine cell cycle profiles on a flow cytometer. The percent of the cells in each cell cycle can be calculated. Cells contacted with a modulator would exhibit, e.g., a higher number of cells that are arrested in G₀/G₁ phase compared to control.

Apoptotic Pathways and Cell Signaling

Additionally, can be analyzed as, for instance, exemplified herein. Screening can done using microarrays.

Tumor Growth in Vivo

Effects of modulators on cell growth can be tested in transgenic or immune-suppressed mice. Knock-out transgenic mice can be made, in which the endogenous gene is disrupted. Such knock-out mice can be used to study effects of the gene and its protein, e.g., as a cancer model, as a means of assaying in vivo for compounds that modulate the protein, and to test the effects of restoring a wild-type or mutant gene to a knock-out mouse.

Knock-out cells and transgenic mice can be made by insertion of a marker gene or other heterologous gene into the endogenous gene site in the mouse genome via homologous recombination. Such mice can also be made by substituting the endogenous gene with a mutated version of the gene, or by mutating the endogenous gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science, 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987). These knock-out mice can be used as hosts to test the effects of various modulators on cell growth.

Alternatively, various immune-suppressed or immune-deficient host animals can be used. For example, genetically athymic “nude” mouse (see, e.g., Giovanella et al., J. Natl. Cancer Inst., 52:921 (1974)), a SCID mouse, a thymectomized mouse, or an irradiated mouse (see, e.g., Bradley et al., Br. J. Cancer, 38:263 (1978); Selby et al., Br. J. Cancer, 41:52 (1980)) can be used as a host. Transplantable tumor cells (typically about 10⁶ cells) injected into isogenic hosts will produce invasive tumors in a high proportions of cases, while normal cells of similar origin will not. Hosts are treated with modulators, e.g., by injection, optionally in combination with other cancer therapeutic agents, including chemotherapy, radiotherapy, immunotherapy or hormonal therapy. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, modulators which are capable, e.g., of inhibiting abnormal cell proliferation or sensitizing tumor cells to cancer therapies, can be identified.

In immune-suppressed or immune-deficient host animals, the inoculating tumor cells preferably overexpress or underexpress the gene or protein of interest. The inoculating tumor cells are also preferably resistant to conventionally used cancer therapies. Exemplified modulators include rituxamib, siRNA, NO donors, NF-κB inhibitors (i.e., dehydroxymethylepoxyquinomicin (DHMEQ)), proteasome inhibitors (i.e., Bortezomib, Velcade), and microtubule inhibitors (i.e., 2-Methoxyestradiol (2ME2) and vincristine). In one example, tumor cells resistant to death receptor-induced (e.g., DR5) apoptosis are inoculated as xenografts in SCID mice. The mice are subsequently treated with one or more inhibitors (siRNA, NO donors, NF-κB inhibitors, etc.) combined with a death receptor agonist (e.g., a monoclonal antibody to DR5 or TRAIL).

Murine, rodent and other animal tumor models for studying cancer are generally described, for example, in Immunodeficient Animals: Models for Cancer Research, Arnold et al., eds., 1996, S Karger Pub; Tumor Models in Cancer Research, Teicher, ed., 2002, Human Press; and Mouse Models of Cancer, Holland, ed., 2004, John Wiley & Sons. Specific murine tumor models for several different cancers have been described, including for example, metastatic colon cancer (Luo, et al., Cancer Cell, 6:297 (2004)), breast cancer (Rahman & Sarkar, Cancer Res, 65:364 (2005)), cholangiocarcinoma (Chen et al., World J Gastroenterol, 11:726 (2005)), and prostate cancer (Tsingotjidou et al., Anticancer Res, 21:971 (2001) and U.S. Pat. No. 6,107,540).

Screening Methods

The present invention also provides methods of identifying compounds that inhibit cancer growth or progression, for example, by inhibiting the binding of a protein to a binding protein or a nucleic acid. The compounds find use in inhibiting the growth of and promoting the regression of a tumor that has altered expression of a protein, for example, prostate cancer, ovarian cancer, lung cancer, renal cancer, breast cancer, colon cancer, leukemias, B-cell lymphomas (e.g., non-Hodgkin's lymphomas, including Burkitt's, Small Cell, and Large Cell lymphomas), hepatocarcinoma or multiple myeloma. The identified compounds can inhibit cancer growth or progression alone, or when used in combination with other cancer therapies, including chemotherapies, radiation therapies, hormonal therapies and immunotherapies.

Using the assays described herein, one can identify lead compounds that are suitable for further testing to identify those that are therapeutically effective modulating agents. One particularly useful assay system utilized a reporter system where a reporter gene (i.e., luciferase or GFP) is operably linked to a promoter sequence comprising a binding sequence. Compounds of interest can be either synthetic or naturally occurring.

Screening assays can be carried out in vitro or in vivo. Typically, initial screening assays are carried out in vitro, and can be confirmed in vivo using cell based assays or animal models. For instance, proteins of the regenerating gene family are involved with cell proliferation. Therefore, compounds that inhibit a protein or nucleic acid can inhibit cell proliferation in comparison to cells unexposed to a test compound. Also, the protein of interest can be involved with tissue injury responses, inflammation, and dysplasia. In animal models, compounds that inhibit the protein can, for example, inhibit wound healing or the progression of dysplasia in comparison to an animal unexposed to a test compound. See,for example, Zhang et al., World J Gastroenter, 9:2635-41 (2003).

Usually, a compound that inhibits the protein is synthetic, but it can also be naturally occurring. The screening methods are designed to screen large chemical or polymer (i.e., inhibitory RNA, including siRNA and antisense RNA, peptides, small organic molecules, etc.) libraries by automating the assay steps and providing compounds from any convenient source to the assays, which are typically run in parallel (e.g., in microtiter formats on microtiter plates in robotic assays).

The invention provides in vitro assays for modulating a protein or nucleic acid in a high throughput format. For each of the assay formats described, “no modulator” control reactions which do not include a modulator provide a background level interaction. In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100- about 1500 different compounds. It is possible to assay many different plates per day; assay screens for up to about 6,000-20,000, and even up to about 100,000-1,000,000 different compounds is possible using the integrated systems of the invention. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

Essentially, any chemical compound can be tested as a potential inhibitor of protein or nucleic acid for use in the methods of the invention. Most preferred are generally compounds that can be dissolved in aqueous or organic (especially DMSO-based) solutions are used. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

Compounds also include those that can regulate transcription and post-transcriptional processing and compounds that can regulate gene expression under the control of a gene or protein of interest. Reporter systems can be used for this analysis.

In one preferred embodiment, inhibitors of protein or nucleic acid are identified by screening a combinatorial library containing a large number of potential therapeutic compounds (potential modulator compounds). Such “combinatorial chemical or peptide libraries” can be screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art (see, for example, Beeler et al., Curr Opin Chem Biol., 9:277 (2005) and Shang and Tan, Curr Opin Chem Biol., 9:248 (2005). Libraries of use in the present invention can be composed of amino acid compounds, nucleic acid compounds, carbohydrates or small organic compounds. Carbohydrate libraries have been described in, for example, Liang et al., Science, 274:1520-1522 (1996) and U.S. Pat. No. 5,593,853.

Representative amino acid compound libraries include, but are not limited to, peptide libraries (see, e.g., U.S. Pat. Nos. 5,010,175; 6,828,422 and 6,844,161; Furka, Int. J. Pept. Prot. Res., 37:487-493 (1991); Houghton et al., Nature, 354:84-88 (1991) and Eichler, Comb Chem High Throughput Screen., 8:135 (2005)), peptoids (PCT Publication No. WO 91/19735), encoded peptides (PCT Publication WO 93/20242), random bio-oligomers (PCT Publication No. WO 92/00091), vinylogous polypeptides (Hagihara et al., J. Amer. Chem. Soc., 114:6568 (1992)), nonpeptidal peptidomimetics with β-D-glucose scaffolding (Hirschmann et al., J. Amer. Chem. Soc., 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT/US96/10287, and Vaughn et al., Nature Biotechnology, 14(3):309-314 (1996)), and peptidyl phosphonates (Campbell et al., J. Org. Chem., 59:658 (1994)).

Representative nucleic acid compound libraries include, but are not limited to, genomic DNA, cDNA, mRNA, inhibitory RNA (RNAi, siRNA) and antisense RNA libraries. See, Ausubel, Current Protocols in Molecular Biology, supra, and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference or siRNA libraries, are reviewed in, for example, Downward, Cell, 121:813 (2005) and Akashi et al., Nat Rev Mol Cell Biol., 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines and dipeptides (Hobbs et al., Proc. Nat. Acad. Sci. USA, 90:6909-6913 (1993)), analogous organic syntheses of small compound libraries (Chen et al., J. Amer. Chem. Soc., 116:2661 (1994)), oligocarbamates (Cho et al., Science 261:1303 (1993)); benzodiazepines (U.S. Pat. No. 5,288,514; and Baum, C&EN, January 18, page 33 (1993)); isoprenoids (e.g., U.S. Pat. No. 5,569,588); thiazolidinones and metathiazanones (e.g., U.S. Pat. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles, (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

Of particular interest are libraries of nitric oxide donor compounds, for example, libraries of molecules with core structures like the nitric oxide donor compounds disclosed in U.S. Pat. Nos. 6,897,218; 6,897,194; 6,780,849; 6,642,260; 6,538,033; 6,451,337; and 5,698,738 (see also, Balogh et al., Comb Chem High Throughput Screen, 8:347 (2005)). Libraries of nitric oxide compounds have been developed by Nitromed of Lexington, Mass.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

Administration and Pharmaceutical Compositions

Molecules and compounds identified that modulate the expression and/or function of protein are useful in treating cancers. Modulators can be administered alone or co-administered in combination with conventional chemotherapy, radiotherapy or immunotherapy.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

Preferred pharmaceutical preparations deliver one or more inhibitors, optionally in combination with one or more chemotherapeutic agents, in a sustained release formulation. Typically, the inhibitor is administered therapeutically as a sensitizing agent that increases the susceptibility of tumor cells to other cytotoxic cancer therapies, including chemotherapy, radiation therapy, immunotherapy and hormonal therapy. In some embodiments, the inhibitor can be an NO donor, including those listed supra, a conjugate comprising NO and another agent (i.e., NO conjugated to aspirin), or an activator of inducible nitric oxide synthase.

In therapeutic use for the treatment of cancer, the compounds utilized in the pharmaceutical method of the invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of cancer diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular vector, or transduced cell type in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

Diagnostic Methods

The present invention also provides methods of diagnosing a cancer, including wild-type, truncated or alternatively spliced forms. Diagnosis can involve determining the level of expression (transcription or translation), DNA binding activity or intracellular localization in a patient and then comparing the level to a baseline or range. Typically, the baseline value is representative expression levels, DNA binding activity or intracellular localization in a healthy person not suffering from cancer. Variation of levels of a polypeptide or polynucleotide of the invention from the baseline range (either up or down) indicates that the patient has a cancer or is at risk of developing a cancer. In some embodiments, the level of expression, DNA binding activity or intracellular localization are measured by taking a blood, urine or tissue sample from a patient and measuring the amount of a polypeptide or polynucleotide of the invention in the sample using any number of detection methods, such as those discussed herein.

Antibodies can be used in assays to detect differential protein expression and protein localization in patient samples, e.g., ELISA assays, immunoprecipitation assays, and immunohistochemical assays. In one embodiment, tumor tissue samples are used in immunohistochemical assays and scored according to standard methods known in the art. PCR assays can be used to detect expression levels of nucleic acids, as well as to discriminate between variants in genomic structure, such as insertion/deletion mutations, truncations or splice variants. Immunohistochemistry and/or immunofluorescence techniques can be used to detect increased nuclear localization of proteins.

In some embodiments, overexpression of protein in a cancerous or potentially cancerous tissue in a patient may be diagnosed or otherwise evaluated by visualizing expression levels and localization in situ of a polynucleotide, a polypeptide, or fragments of either. Those skilled in the art of visualizing the presence or expression of molecules including nucleic acids, polypeptides and other biochemicals in the tissues of living patients will appreciate that the gene expression information described herein may be utilized in the context of a variety of visualization methods. Such methods include, but are not limited to, single-photon emission-computed tomography (SPECT) and positron-emitting tomography (PET) methods. See, e.g., Vassaux and Groot-wassink, “In Vivo Noninvasive Imaging for Gene Therapy,” J. Biomedicine and Biotechnology, 2:92-101(2003).

PET and SPECT imaging shows the chemical functioning of organs and tissues, while other imaging techniques—such as X-ray, CT and MRI—show structure. The use of PET and SPECT imaging is useful for qualifying and monitoring the development of cancers and/or therapy resistant cancers, including prostate cancer, ovarian cancer, lung cancer, renal cancer, breast cancer, colon cancer, leukemias, B-cell lymphomas, myelomas and hepatocarcinomas. In some instances, the use of PET or SPECT imaging allows diseases to be detected years earlier than the onset of symptoms. The use of small molecules for labelling and visualizing the presence or expression of polypeptides and nucleotides has had success, for example, in visualizing proteins in the brains of Alzheimer's patients, as described by, e.g., Herholz, K. et al., Mol Imaging Biol., 6(4):239-69 (2004); Nordberg, A., Lancet Neurol., 3(9):519-27 (2004); Zakzanis, K. et al., Neuropsychol Rev., 13(1):1-18 (2003); Kung M. et al., Brain Res., 1025(1-2):98-105 (2004); and Herholz, K., Ann Nucl Med., 17(2):79-89 (2003).

A polypeptide, a polynucleotide, or fragments of either, can be used in the context of PET and SPECT imaging applications. After modification with appropriate tracer residues for PET or SPECT applications, molecules which interact or bind with a transcript or with any polypeptides encoded by those transcripts may be used to visualize the patterns of gene expression and facilitate diagnosis of cancers.

Compositions, Kits and Integrated Systems

The invention provides compositions, kits and integrated systems for practicing the assays described herein using polypeptides or polynucleotides of the invention, antibodies specific for polypeptides or polynucleotides of the invention, etc.

The invention provides assay compositions for use in solid phase assays; such compositions can include, for example, one or more polynucleotides or polypeptides of the invention immobilized on a solid support, and a labeling reagent. In each case, the assay compositions can also include additional reagents that are desirable for hybridization. Modulators of expression or activity of polynucleotides or polypeptides of the invention can also be included in the assay compositions.

The invention also provides kits for carrying out the therapeutic and diagnostic assays of the invention. The kits typically include one or more probes that comprises an antibody or nucleic acid sequence that specifically binds to polypeptides or polynucleotides of the invention, and a label for detecting the presence of the probe. The kits can find use, for example for measuring the levels of protein or transcripts, or for measuring DNA-binding activity. The kits may include several polynucleotide sequences encoding polypeptides of the invention. Kits can include any of the compositions noted above, and optionally further include additional components such as instructions to practice a high-throughput method of assaying for an effect on expression of the genes encoding the polypeptides of the invention, or on activity of the polypeptides of the invention, one or more containers or compartments (e.g., to hold the probe, labels, or the like), a control modulator of the expression or activity of polypeptides of the invention, a robotic armature for mixing kit components or the like.

The invention also provides integrated systems for high-throughput screening of potential modulators for an effect on the expression or activity of the polypeptides of the invention. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety. A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT®, WINDOWS95®, WINDOWS98®, or WINDOWS2000® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

### EXAMPLES

The following examples are offered to illustrate, but not to limit the claimed invention.

### Example 1

### Regulation of Chemoresistance and Immune Resistance of B-NHL Cell Lines by Overexpression of YY1 and Bcl-xl, Respectively: Reversal of Resistance by Rituximab

We have recently reported that treatment of B-Non-Hodgkin's Lymphoma (NHL) cell lines with rituximab (anti-CD20 antibody) sensitizes the tumor cells to both chemotherapy and Fas-induced apoptosis (Jazirehi and Bonavida, Oncogene, 24:2121-2145 (2005)). This study investigated the underlying molecular mechanism of rituximab-mediated reversal of immune and drug resistance. Treatment of B-NHL cell lines inhibited the constitutively activated NF-κB. Cells expressing dominant active IκB or treated with NF-κB specific inhibitors were sensitized to both drugs and FasL agonist mAb (CH-11)-induced apoptosis. Downregulation of Bcl-_{XL} expression via inhibition of NF-κB activity correlated with chemosensitivity. The direct role of Bcl-_{XL} in chemoresistance was demonstrated by the use of BC1-_{XL} overexpressing Ramos cells, Ramos HA-Bcl-_{XL} (gift from Genhong Cheng, UCLA), which were not sensitized by rituximab to drug-induced apoptosis. However, inhibition of Bcl-_{XL} in Ramos HA-Bcl-x resulted in sensitization to drug-induced apoptosis. The role of Bcl-_{XL} expression in the regulation of Fas resistance was not apparent as Ramos HA-Bcl-_{XL} cells were as sensitive as the wild type cells to CH-11-induced apoptosis. Several lines of evidence support the direct role of the transcription repressor Yin-Yang 1 (YY1) in the regulation of resistance to CH-11-induced apoptosis. Inhibition of YY1 activity by either rituximab, the NO donor DETANONOate, or following transfection with YY1 siRNA all resulted in upregulation of Fas expression and sensitization to CH-11-induced apoptosis. These findings show two complementary mechanisms underlying the chemo-sensitization and immuno-sensitization of B NHL cells by rituximab via inhibition of NFκB. The regulation of chemoresistance by NFκB is mediated via Bcl-_{XL} expression whereas the regulation of Fas resistance by NF-B is mediated via YY1 expression and activity. These findings show that drug-resistant NHL tumor cells are sensitive to immune-mediated therapeutics.

### Example 2

### Rituximab-Mediated Inhibition of the Transcription Repressor Yin-Yang 1 (YY1) in NHL B Cell Lines: Upregulation of Fas Expression and Sensitization to Fas-Induced Apoptosis

We have reported that rituximab triggers and inhibits anti-apoptotic gene products in NHL B-cell lines resulting in sensitization to drug-induced apoptosis (Alas et al., Clin. Cancer Res., 8:836 (2001); Jazirehi et al., Mol. Cancer Therapy, 2:1183 ( 2003); Vega et al., Oncogene, 23:3530 (2004)). This study investigated whether rituximab also modifies intracellular signaling pathways resulting in the sensitization of NHL cells to Fas-induced apoptosis. Treatment of the NHL cell lines (2F7, Ramos, and Raji) with rituximab (20 μg/ml) sensitized the cells to CH-11 (FasL agonist mAb)-induced apoptosis and synergy was achieved. Fas expression was up-regulated by rituximab as early as 6 h post treatment as determined by flow cytometry, RT-PCR, and Western. Rituximab inhibited both the expression and activity of the transcription repressor Yin-Yang 1 (YY 1) that negatively regulates Fas transcription. Inhibition of YY1 resulted in upregulation of Fas expression and sensitization of the tumor cells to CH-11-induced apoptosis. Downregulation of YY1 expression was the result of rituximab-induced inhibition of both the p38MAPK signaling pathway and constitutive NF-κB activity. The dual roles of NF-κB and YY1 in the regulation of Fas expression were corroborated by the use of a dominant-active inhibitor of NF-κB (Ramos IκB-ER mutant) and YY1 siRNA, respectively. The role of rituximab-mediated inhibition of the p38MAPK/NF-κB/YY1 pathways, which result in both Fas upregulation and sensitization to CH11-induced apoptosis, was corroborated by the use of specific chemical inhibitors directed at various components of these pathways. Rituximab-mediated sensitization to CH-11-induced apoptosis was executed through the Type II mitochondrial apoptotic pathway. Altogether, these findings provide a novel mechanism of rituximab-mediated signaling by inhibiting the p38MAPK/NF-κB/YY1 pathways and resulting in the sensitization of B NHL to Fas-induced apoptosis. These findings show an additional mechanism of rituximab-mediated effect in vivo in addition to complement-dependent cytotoxicity (CDC) and antibody-dependent cellular cytotoxicity (ADCC).

### Example 3

### Rituximab Diminishes the Constitutive Activity of the PI3k-Akt Signaling Pathway in Ramos B-NHL Cells

Rituximab (chimeric anti-CD20 monoclonal antibody) is currently being used, alone or in combination with chemotherapy, in the treatment of B-Non Hodgkin's Lymphoma (B-NHL). We have reported that rituximab treatment of B-NHL cell lines sensitizes the drug-resistant tumor cells to apoptosis by various chemotherapeutic drugs and chemosensitization was due, in large part, to the selective inhibition of the anti-apoptotic Bcl-_{XL} gene product. The constitutive activation of the Akt pathway in B-NHL results in overexpression and functional activation of Bcl-_{XL}. The hypothesis that the rituximab-induced inhibition of Bcl-_{XL} expression and chemosensitization resulted, in part, from its inhibitory activity of the Akt pathway was tested using the drug-resistant Ramos B-NHL cell line. Time kinetic analysis revealed that treatment of Ramos with rituximab inhibited phophorylation of Akt (p-Akt), but not unphosphorylated Akt and the inhibition was first detected at 3 to 6 h post-rituximab treatment. Similar time kinetics revealed rituximab-induced inhibition of p-PDK1, p-Bad, p-IKKα/β and p-Iκβα and no inhibition of unphosphorylated proteins. In addition, rituximab treatment resulted in significant increase of Bcl-_{XL} -Bad heterodimeric complexes as compared to untreated cells. The role of the Akt pathway in the regulation of resistance was corroborated by the use of the Akt inhibitor, LY294002, and by transfection with siRNA Akt. Treatment of Ramos with LY294002 resulted in inhibition of Bcl-_{XL} expression and sensitization of rituximab sensitive and rituximab resistant Ramos cells to both CDDP and ADR-induced apoptosis. Further, transfection of Ramos with Akt siRNA, but not control siRNA, inhibited both Akt and Bcl-_{XL} expression and sensitized the cells to CDDP-induced apoptosis. The present findings demonstrate for the first time that rituximab inhibits the constitutively activated Akt pathway in Ramos and its inhibition contributes to sensitization of the drug resistant cells to apoptosis by chemotherapeutic drugs. The findings also identify the Akt pathway as target for therapeutic intervention in the reversal of rituximab and drug resistant B-NHL.

Materials and Methods

Reagents

RPMI-1640, opti-MEM and fetal bovine serum (FBS) were purchased from Gibco (Grand Island, N.Y., USA). Rituximab (stock, 10 mg/mL) was obtained commercially. Rituximab (Fab)′₂ was a generous gift from Dr. Chin (Idec Biogen, San Diego). LY294002, anti Lyn, anti-phospho Lyn (Tyr507), anti-IKKα/β, anti-IKbα/β, anti-IKKβ, anti-Bcl-_{XL}, anti-Bad, anti-phospho-Bad (Ser136), anti-phopho PDK1, anti-human phospho-Akt (Ser473, Thr308), anti-Akt, and anti-β-actin antibodies were obtained from Cell Signaling Technology (Beverly, Mass., USA). Akt siRNA and control scramble siRNA were purchased from Sigma (Mo., USA). Protein A-agarose was purchased from Pierce (Rockford, Ill.).

Cell Culture

The CD20+human Burkitt's lymphoma B-cell line Ramos was obtained from ATCC (Manassas, Va., USA). Cells were cultured in 50 ml culture flasks (Costar, Cambridge, Mass.) at 37° C. in an atmosphere of 5% CO₂ in RPMI 1640 supplemented with 10% (v/v) heat-inactivated FBS (to ensure the absence of complement), penicillin/streptomycin, non essential amino acid and 2-mercaptoethanol. The Ramos RR1 clone was generated in our laboratory following culture of Ramos in the presence of increasing concentrations of rituximab for 10 weeks and clones were isolated by limiting dilution (Jazirehi et al., 2006).

Drug Pretreatment

Ramos B-NHL cells (1×10⁶ cells/ml) were grown in complete medium in 50 ml tissue culture flasks and were treated with a previously established optimal concentration (20 ug/ml) of rituximab for 18 h. The cells were then washed and fresh medium was added and seeded into 12 well plates (Costar, Cambridge, Mass.). Indicated concentrations of drugs (CDDP, ADR) were then added, and the cells were incubated for another 24 h for maximal cytotoxicity. At the end of the incubation period, the cells were harvested and subjected to propidium iodide (PI) (Molecular Probes, Leiden, Netherlands) staining according to the specifications of the PI staining kit (Roche Diagnostics Corporation, Indianapolis, Ind.) and evaluated by flow cytometric analysis. LY294002 was dissolved in DMSO (Sigma-Aldrich) and added up to 50 uM.

Analysis of Apoptosis

DNA fragmentation was detected by flow cytometric analysis after propidium iodide staining (Alas, S. et al., Cancer Res., 61:5137-44 (2001)). Cells with fragmented DNA were considered as apoptotic. Fixation and staining of the cells were performed according to standard protocols. Briefly, samples containing 1×10⁶ cells were collected, resuspended in ice-cold PBS and fixed with ethanol (70% v/v). After 50 min incubation at 4° C., cells were centrifuged and incubated with 50 ug/ml of PI for 20 min at 37° C. Fluorescence was measured on a FACScan flow cytometer (BD Immnunocytometry Systems) within 1 h. A minimum of 10,000 events was collected on each sample and acquired in listmode by a PC Pentium computer. Cellular debris were excluded from analysis by raising the forward scatter threshold, and the DNA content of the intact nuclei was recorded on a logarithmic scale. The percentage of apoptotic cells is represented as the percentage of hypodiploid cells accumulated at the sub-GO phase of the cell cycle.

Western Blotting

After stimulation, cells (2×10⁶ cells) were collected and resuspended with lysis buffer (20 mM Tris-HCl (pH 8.0), 2 mM EDTA, 3% NP-40, 100 mM NaCl, 50 mM NaF, 1 uM PMSF, 1 uM V04, 5 ug/ml aprotinin, 5 ug/ml leupeptin). After 30 min of incubation on ice, samples were centrifuged at 10,000×g, for 10 min at 4° C. to remove cellular debris. The concentration of the protein in the resulting supernatant was measured using the Bio-Rad protein assay reagent and equalized with SDS-PAGE buffer (0.125 M Tris-HCI pH 6.8, 4% SDS, 20% glycerol, 10% 2-mercaptoethanol, bromophenolblue).Then equal amounts of proteins were separated using sodium dodecylsulphate-polyacrylamide gel electrophoresis (10-12% SDS-PAGE) with a Mini-Protean system (Bio-Rad, Hercules, Calif., USA) and electrophoretically transferred to nitrocellulose membranes. The membranes were blocked for 30 min with 5% non-fat dry milk diluted in 0.05% Tween-20 Tris-buffered saline at room temperature and incubated overnight with the primary antibodies. After incubation with the primary antibody against Akt (1:300), Bad, PDK1, (1:500), phospho-specific antibodies against phospho-Akt (Ser 473, Thr 308) (1:300), phospho-PDK1 and phospho-Bad (Ser136) (both 1:300) for overnight, the membranes were washed and incubated with HRP-coupled secondary antibodies (Cell Signaling, Mass., USA). Immunoreactive bands were detected using an enhanced chemiluminiscence system (Amersham Pharmacia Biotech, UK).

Immunoprecipitation

Ramos cells (2×10⁶ cells) were collected and resuspended with lysis buffer (20 mM Tris-HCl (pH 8.0), 2 mM EDTA, 3% NP-40, 100 mM NaCl, 50 mM NaF, 1 uM PMSF, 1 uM V04, 5 ug/ml aprotinin, 5 ug/ml leupeptin). After 30 min of incubation on ice, samples were centrifuged at 10000×g, for 10 min at 4° C. to remove cellular debris. The concentration of the protein in the resulting supernatant was measured using the Bio-Rad protein assay reagent and equalized with RIPA buffer. One mg of total lysate was incubated with 5 μl protein A agarose beads (Pierce, Rockford, Ill.), centrifuged and the supernatant was recovered. The supernatant was mixed with 15 μl of protein A agarose and 2 μg Bcl-_{XL} mouse monoclonal antibody or normal mouse IgG-1 at 4° C. for 4 h. Following centrifugation, the beads were washed 4× with RIPA buffer and the beads were boiled in 50 μl Laemmli buffer and subjected to SDS-polyacrylamide gel electrophoresis and blotted with rabbit anti-Bad antibody. After overnight incubation with the primary antibody, the membranes were washed and incubated with HRP-coupled secondary goat anti-rabbit antibody (Cell Signaling, Mass., USA). Immunoreactive bands were detected using an enhanced chemiluminiscence system (Amersham Pharmacia Biotech, UK).

Transfection of Ramos with Akt siRNA

Ramos B-NHL cells were cultured in 1 ml of Opti-MEM medium without antibiotics and FBS. Transfections were performed using Xtreme GENE reagent (Roche Diagnostics Corporation, Indianapolis, Ind.), Akt siRNA (Sigma, Mo., USA) and control scramble siRNA (Sigma, Mo., USA) according to the manufacturers' instructions. Briefly, 4 ul of Akt siRNA or a scramble control siRNA solution mixed with 96 ul of opti-MEM was incubated with 10 ul of the transfection reagent in 90 ul of serum-free Opti-MEM medium for 15 min to facilitate complex formation. The resulting mixture was gently added to Ramos cells cultured in a 12 well plate with 1 ml of medium. Akt, phospho-Akt and Bcl-_{XL} levels were determined by Western blotting. To determine Akt siRNA-induced sensitization to CDDP-induced apoptosis, following transfection the cells were treated with CDDP for 24 h, then fixed and permeabilized for PI staining, and analyzed by flow cytometry as described above.

Statistical Analysis

Assays were set up in triplicates and the results were expressed as the mean±SD. Statistical analysis and P value determinations were done by two-tailed paired t test with a confidence interval of 95% for determination of the significance of differences between the treatment groups. P<0.05 was considered to be significant.

### Example 3A

### The Effects of Rituximab Treatment on the Activity of the Akt Pathway

Rituximab treatment of B-NHL cell lines results in inhibition of the survival Raf-1/MEK/ERK1/2 and the NF-κB signaling pathways both of which led to the selective inhibition of the expression of the anti-apoptotic gene product Bcl-_{XL}. This results in the sensitization of drug-resistant B-NHL tumor cells to apoptosis by various chemotherapeutic drugs. Here, other signaling pathways that also regulate Bcl-_{XL} expression and/or activity were explored as to whether they may also be inhibited by rituximab. The Akt pathway which is constitutively activated in many cancers in part by Src kinases and PI3K (Inabe, K. et al., Blood, 99:584-9 (2002); Tedder, T. et al., Biochemical Society Transactions, 30:807-811 (2002)) has been shown to regulate Bcl-_{XL} transcriptional and post-transcriptional expression and activity (Hayakawa, J. et al., Cancer Res., 60:5988-94 (2000); Weintraub, S. et al., Cancer Cell, 5:3-4 (2004); Castilla, C. et al., Endocrinology [Epub ahead of print] (2006)).

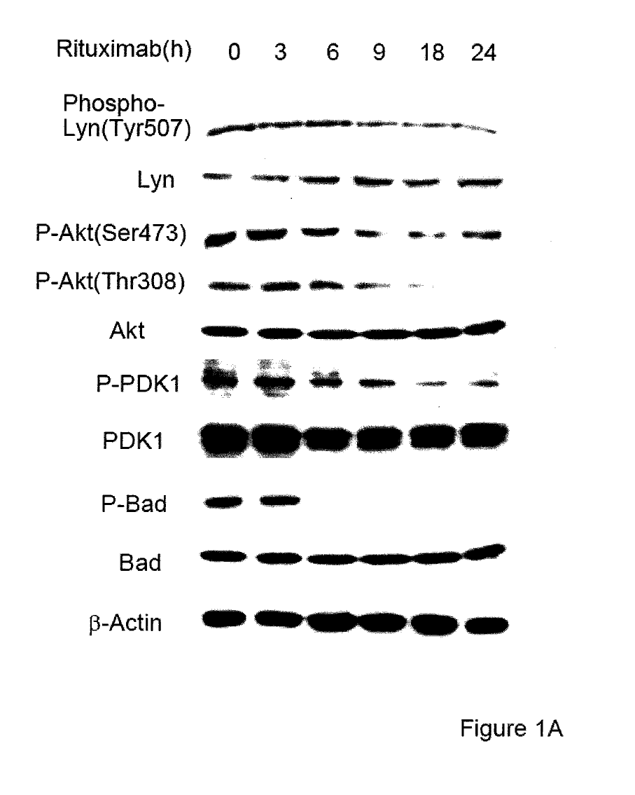

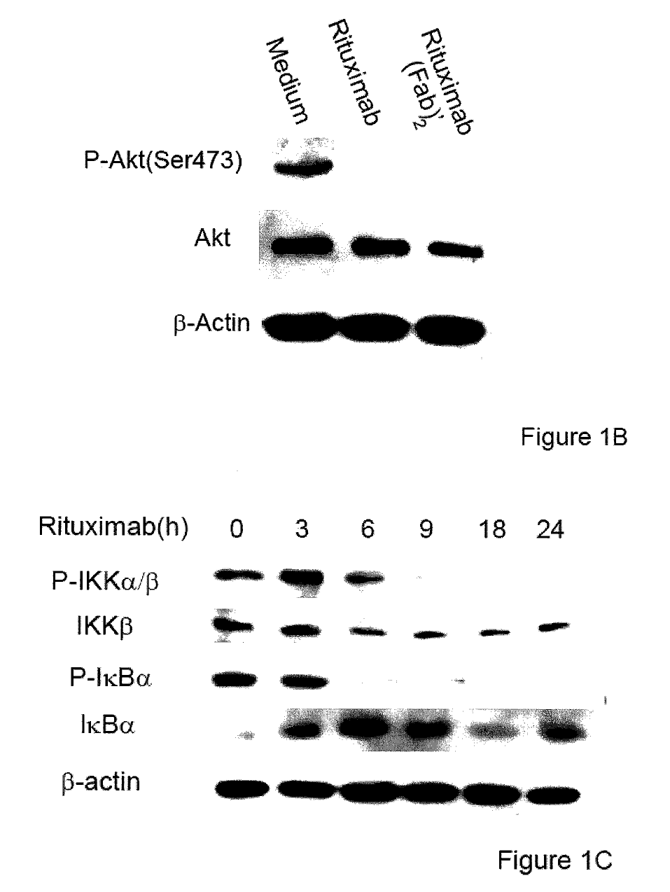

Ramos cells were treated with rituximab (20 μg/ml) for different time periods (0-24 h) and cell lysates were prepared for analysis of various proteins of the Akt signaling pathway (non-phosphorylated and phosphorylated proteins) by western. Time kinetic analyses revealed that rituximab treatment of Ramos inhibited p-Lyn, p-Akt (Ser473 and Thre308), p-PDK1 and p-Bad while there was no detectable inhibition of non-phosphorylated proteins. The inhibition was first observed at 3-6 h post rituximab treatment (FIG. 1A). The rituximab-mediated inhibition of p-Akt was a direct effect of rituximab interaction with CD20 as treatment of Ramos with Fc-devoid Rituximab (Fab′)₂ also inhibited p-Akt like rituximab. This finding ruled out any contribution of the Fc in rituximab-mediated cell signaling (FIG. 1B).

The PI3K-Akt signaling pathway regulates the transcriptional expression of Bcl-_{XL} via IKK, IκB and NF-κB activation (Sugimori, K. et al., J Bone Miner Metab., 23:411-9 (2005)). Whether rituximab treatment of Ramos could inhibit the phosphorylation of these gene products was next investigated. This was analyzed by examining total cell lysates of Ramos cells that were treated with rituximab for different time periods (1-24 h) and thereafter they were subjected to analysis by western using phospho-specific and non-phospho-specific antibodies. Treatment of Ramos cells with rituximab diminished the levels of p-IKKα/β and p-Iκβα, but not the levels of non-phosphorylated proteins and the inhibition was first detected at 3-6 h post-rituximab treatment and significant inhibition was observed after 24 h (FIG. 1C).

### Example 3B

### Decreased Bcl-_{xL} Activity and Expression by Rituximab-mediated Inhibition of the Akt Pathway

The above findings demonstrated that rituximab treatment of Ramos cells diminishes the activity of the constitutively activated Akt pathway. The Akt signaling pathway regulates Bcl-_{XL} activity via phosphorylation of Bad, which normally dissociates from Bcl-_{XL} and dissociated Bcl-_{XL} can thus exert its anti-apoptotic activity. Likewise, phosphorylation of Bad can also promote cell survival (Datta, S. et al., Genes & Development, 13:2905-2927 (1999); Hayakawa, J. et al., Cancer Res., 60:5988-94 (2000)). We next investigated whether the constitutive activation of the Akt pathway in Ramos cells could lead to phosphorylation of Bad and therefore, unbound cytosolic Bcl-_{XL} which can exert its anti-apoptotic activity.

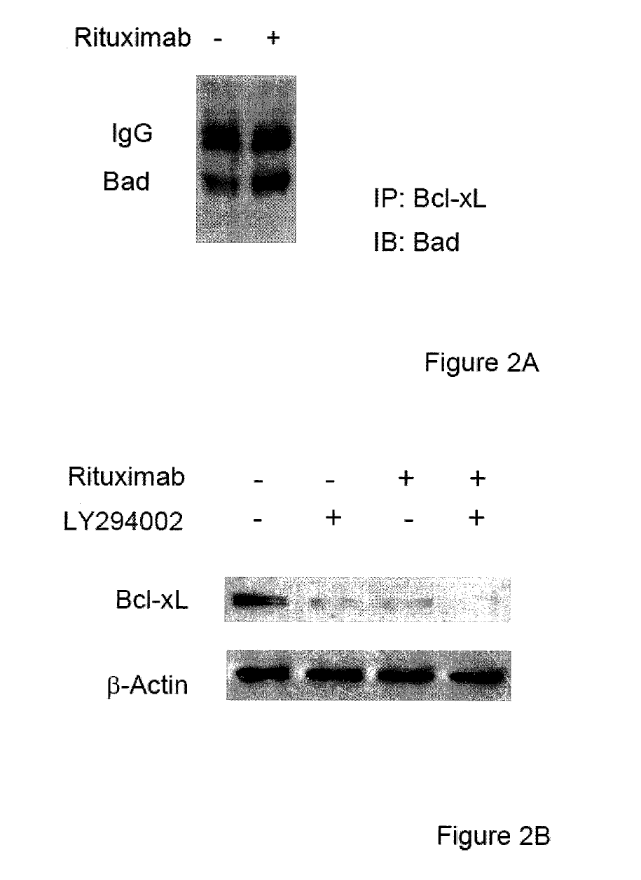

Treatment of Ramos with rituximab, which inhibited phospho-Bad (FIG. 1A), was found to significantly enhance the physical association of Bad and Bcl-_{XL}. This was determined by treating Ramos cell lysates with anti-Bcl-_{XL} antibody and the immuno-precipitated complexes were analyzed for Bad by western. It is clearly shown in FIG. 2A that treatment of Ramos cells with rituximab significantly augmented the association of Bad with Bcl-_{XL} as compared to untreated cells. These findings demonstrate that treatment of Ramos with rituximab inhibited the Akt signaling pathway and enhanced the complex formation of non-phosphorylated Bad with Bcl-_{XL} and hence, reducing the anti-apoptotic activity of Bcl-_{XL}.

Treatment of Ramos cells with rituximab significantly inhibited Bcl-_{XL} expression via inhibition of the ERK 1/2 and NF-κB pathways confirming our previous findings (Jazirehi, A. et al., Cancer Res., 65:264-76 (2005)) (FIG. 2B). We examined the direct role of the Akt pathway in the regulation of Bcl-_{XL} expression. Ramos cells were treated with the Akt specific inhibitor, LY294002, and cell lysates were examined for levels of Bcl-_{XL} . Treatment of Ramos cells with LY294002 significantly inhibited Bcl-_{XL} expression and mimicked rituximab mediated inhibition of Bcl-_{XL} expression (FIG. 2B). These findings suggested that the Akt pathway may be involved in rituximab-mediated inhibition of Bcl-_{XL} function.

### Example 3C

### Participation of Rituximab-mediated Inhibition of the Akt Pathway in the Sensitization of Ramos Cells to Drug-induced Apoptosis

Two approaches were examined to determine the role of the AKT pathway in chemosensitization, namely, (i) the use of a pharmacologic specific Akt inhibitor, Ly294002, and (ii) inhibition of Akt by siRNA.

(i) Inhibition of the Akt pathway by LY294002 mimics rituximab-mediated sensitization of Ramos cells to both CDDP and ADR-induced apoptosis.

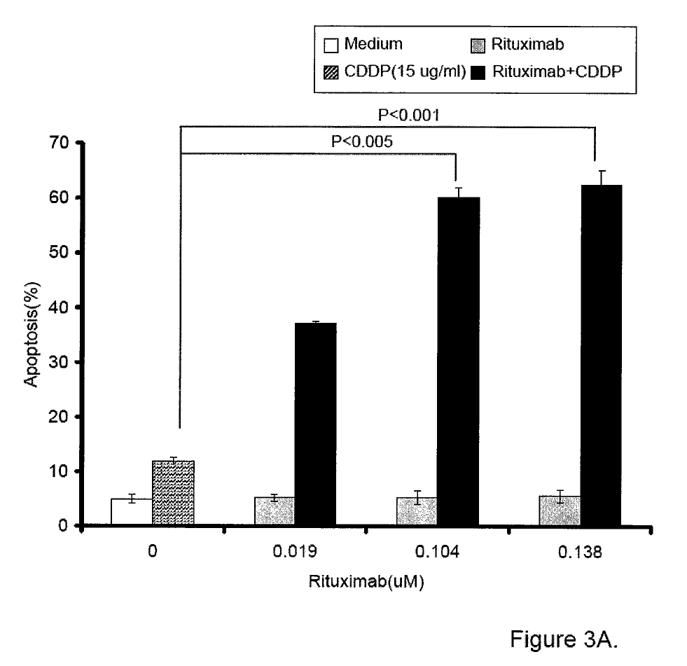

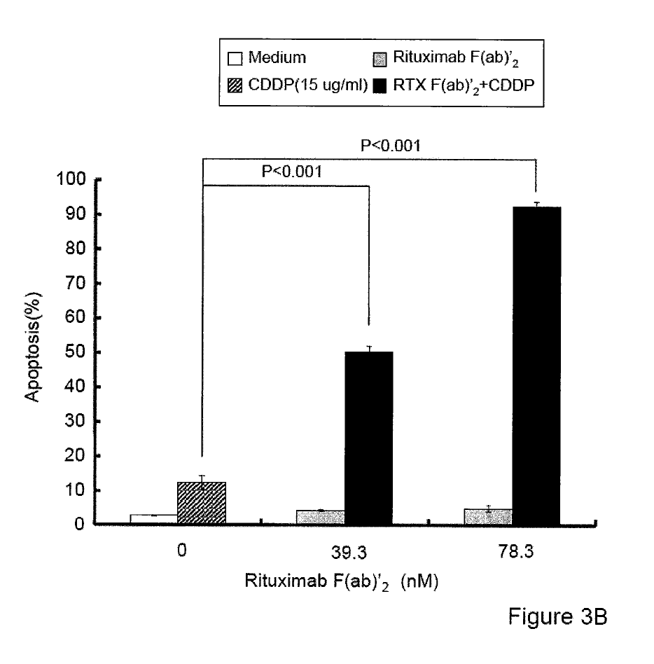

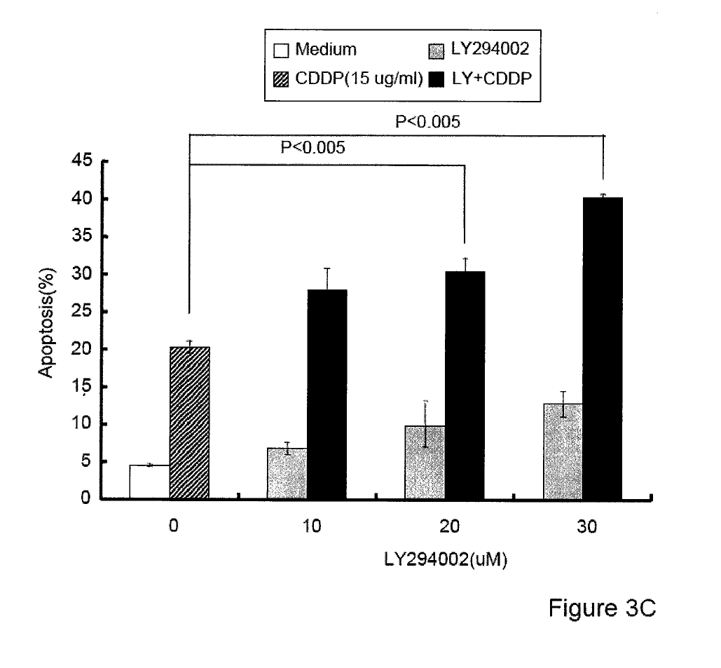

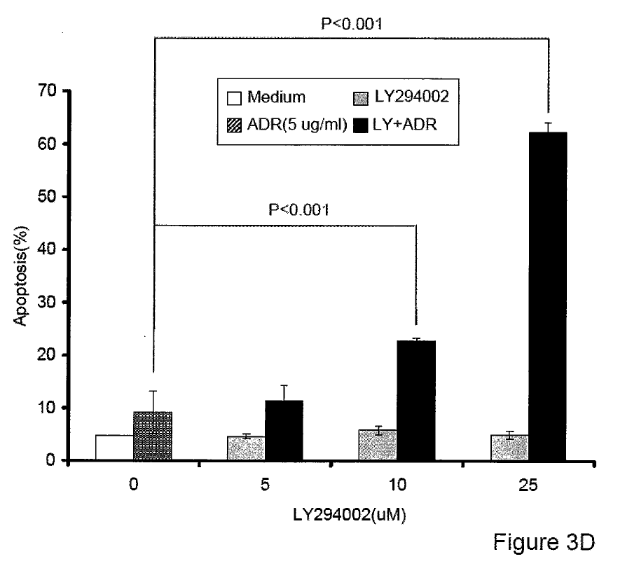

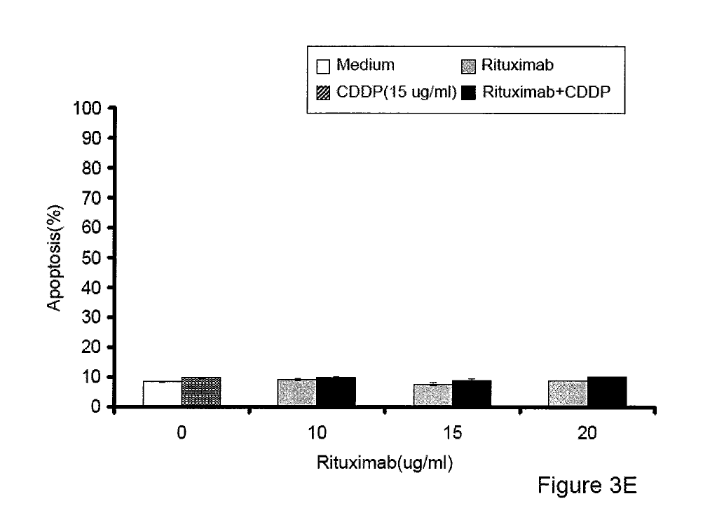

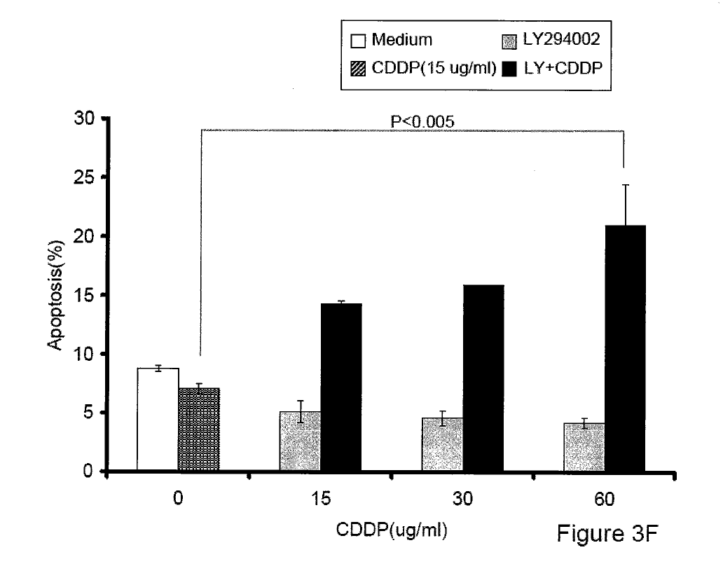

Rituximab-mediated inhibition of the ERK1/2 and NF-κB pathways and Bcl-_{XL} expression can overcame drug-resistance and sensitize the tumor cells to apoptosis by various chemotherapeutic drugs. Here, we show, rituximab and rituximab-Fab′₂ treatment of Ramos cells for 24 h sensitize the cells significantly to CDDP-induced apoptosis, respectively, and the sensitization was a function of the antibody concentration used. Treatment with single agent had no cytotoxic activity (FIG. 3A,B). By comparison, treatment of Ramos cells with various concentrations of LY294002 also sensitized the cells to CDDP-induced apoptosis and the sensitization was a function of the LY294002 concentration used (FIG. 3C). Similar findings were observed for LY294002-mediated sensitization of Ramos cells to ADR-induced apoptosis (FIG. 3D). In addition to the wild-type, we have also examined the effect of LY294002 in the sensitization of rituximab-resistant Ramos clone (Ramos RR1). Treatment of Ramos RR1with rituximab did not sensitize the cells to CDDP-induced apoptosis (FIG. 3E); however, treatment with LY294002 resulted in significant sensitization of Ramos RR1 to CDDP-induced apoptosis (FIG. 3F).

(ii). Sensitization of Ramos cells to drug-induced apoptosis by transfection with Akt siRNA.

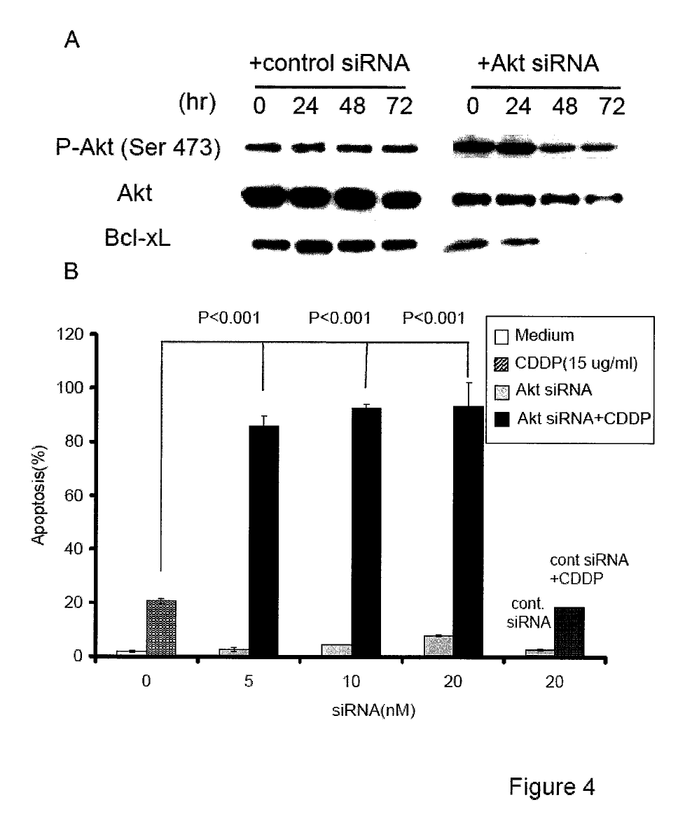

The direct role of the Akt pathway in the regulation of drug resistance was tested by transfecting Ramos cells with Akt siRNA as described in methods. The specificity of transfection was determined by using a control siRNA. Treatment of Ramos cells with Akt siRNA resulted in significant inhibition of p-Akt and Akt in a time-dependent manner. The inhibition of Akt by Akt siRNA resulted in inhibition of Bcl-_{XL} expression; however, transfection with control siRNA had no effect (FIG. 4A). The cells tranfected with siRNA were then examined for sensitivity to CDDP-induced apoptosis. Since the findings above demonstrated that Ly294002-induced inhibition of Akt sensitized the cells to both CDDP and ADR-induced apoptosis, we tested whether inhibition of Akt by siRNA could also sensitize Ramos cells to drug-induced apoptosis. Indeed, treatment with Akt siRNA, but not with siRNA control, sensitized the cells to CDDP-induced apoptosis (FIG. 4B).

Altogether, the above findings demonstrate that rituximab-induced inhibition of the Akt pathway contributes to rituximab-mediated sensitization of rituximab sensitive and rituximab resistant Ramos cells to drug-induced apoptosis.

The present findings demonstrate for the first time that treatment of the B-NHL cell line Ramos with rituximab resulted in significant inhibition of the constitutively activated PI3k/Akt signaling pathway. Rituximab-mediated inhibition of this pathway resulted in both inhibition of Bcl-_{XL} activity through the increased formation of complexes between Bad and Bcl-_{XL} as well as down regulation of Bcl-_{XL} expression. Rituximab sensitized the drug-resistant Ramos cells to apoptosis by chemotherapeutic drugs such as CDDP and adriamycin. The role of the Akt pathway in the regulation of chemoresistance was corroborated by both the use of the PI3K inhibitor Ly294002 and by the use of silencer RNA for Akt, both of which inhibited Bcl-_{XL} expression and sensitized the tumor cells to drug-induced apoptosis. These findings establish in B-NHL the involvement of the Akt pathway in chemoresistance and whose inhibition by rituximab results in chemosensitivity. In addition, the present findings identify the Akt pathway as a novel target for therapeutic intervention and inhibitors of this pathway can be used alone or in combination with drugs in the treatment of rituximab and drug resistant B-NHL.

Aberrant activation of the PI3K-Akt pathway has been widely implicated in many cancers. PI3K is a signaling component downstream of growth factor receptors tyrosine kinases (RTKs) (Cantley, L., Science, 296:1655-7 (2002)). The PI3K-Akt signaling pathway regulates many normal cellular processes including cell proliferation, survival, growth and motility processes that are critical for tumorigenesis (Vivanco, I. et al., Nat Rev Cancer, 2:489-501 (2002)). Hyperactivation of the PI3K-Akt pathway is often genetically selected during tumorigenesis and the normal cellular functions regulated by this pathway are recruited to promote proliferation and survival of cancer cells. The PI3K-Akt pathway is a key regulator of cell survival through multiple downstream targets. PI3K phosphorylates PIP2 at the 3′ position on the inositol ring and converts PiP2 to PiP3. Subsequently, PIP3 recruits other downstream molecules, particularly the serine-threonine kinases Akt and PDK-1 via binding to their pleckstrin homology (PH) domains. Akt is partially activated through phosphorylation at serine 308 in its activation loop by PDK-1. Additional phosphorylation of serine 473 in the C-terminus of Akt results in its full activation. Ramos cells exhibit constitutively activated signaling of the Akt pathway. Rituximab treatment inhibited both pAkt (ser473) and pAkt (Thr308) 3-6 hours post treatment and the inhibition was augmented as a function of time up to 24 hours. There was no inhibition of non phosphorylated Akt. The inhibition pPDK1 by rituximab followed the same time kinetics as those for Akt. Akt can phosphorylate the Bcl-2 family member Bad, causing its sequestration from the mitochondrial membrane by 14-3-3 protein (Datta, S. et al., Genes & Development, 13:2905-2927 (1999)). Bad is a member of the family of Bcl-2 proteins functioning as apoptosis regulatory factors. Bad in its unphosphorylated form binds to and inactivates anti-apoptotic proteins such as Bcl-2 and Bcl-_{XL} leading to its pro-apoptotic function. Recently, Akt has been shown to phosphrylate Bad at serine 136 which allows Bad to dissociate from the Bcl-2/Bcl-_{XL} complex and loses its pro-apoptotic function (Henshall, D. et al., J Neurosci., 22:8458-65 (2002); Kamada, H. et al., J Cereb Blood Flow Metab. [Epub ahead of print] (2006)). Inhibition of pBad was initially observed at 6 hour post rituximab treatment in the absence of inihibition of non-phosphorylated Bad. The inhibition of pBad in Ramos resulted in the augmented association of Bad with Bcl-_{XL} to form more complexes. Complexes of Bad and Bcl-2 could not be detected in Ramos since these cells are deficient in Bcl-2 expression and we have previously confirmed this finding (Jazirehi, A. et al., Cancer Res., 64:7117-26 (2004)).

Our data showed that in Ramos, aside from the proteins responsible for the activation of the Akt pathway, IKK and IKB were constitutively activated. We have previously reported that rituximab treatment sensitized NHL cells to drug induced-apoptosis via inhibition of NFκB. A cross talk between the PI3K and the NFκB pathway has been previously reported in a number of systems (Yin, D. et al., J Neuroimmunol., 174:101-7 (2006); Fishman, P. et al., Arthritis Res Ther, 8:R33 (2006)). As shown in this study, treatment with rituximab inhibited the phosphorylation of IKK and IκBα indicating that rituximab inhibits the anti-apoptotic effect regulated by NFκB. Considerable controversy remains regarding the involvement of Akt in signal-induced IKK activation. Studies by Ozes, O. et al., Nature, 401:82-5 (1999) indicated that Akt was required for TNFα or G protein activation induced NFκB activation by directly phosphorylating and activating IKKα in tumor cell lines. Chandramohan, V. et al., J Immunol., 172:5522-7 (2004) showed in B-lymphoma cells generated from mice that inhibition of the PI3K/Akt signaling pathway resulted in decreased level of NFκB. Thus, it is possible that Akt phosphorylates IKK in a cell context dependent and stimulation-dependent manner.

The direct role of the activated Akt pathway in the regulation of resistance was independently demonstrated by the use of a pharmacologic inhibitor and by siRNA. Both inhibited Bcl-_{XL} expression and sensistized the tumor cells to drug-induced apoptosis. Ly294002 has long been used as a selective inhibitor of PI3K-mediated phosphorylation of Akt (Poh, T. et al., Cancer Res., 65:6264-74 (2005)). The biological activity mediated by Ly294002 and its relevance to apoptosis has been attributed to its effect on the PI3K/Akt survival network. Ly294002 is a flavonoid therapeutic; alone it has anti-proliferative and pro-apoptotic activities (Wetzker, R. et al., Curr Pharm Des., 10:1915-22 (2004)). Administration of Ly294002 in mice bearing human xenograft inhibited tumor growth and induced apoptosis (Semba, S. et al., Cancer Res., 8:1957-63 (2002); Fan, Q. et al., Cancer Res., 63:8930-8 (2003)). The combinational treatment with cytotoxic drugs enhances the effectiveness of the treatment (Wetzker, R. et al., Curr Pharm Des., 10:1915-22 (2004)). Liu, X. et al., Mol Cancer Ther., 5:494-501(2006) examined the role of the PI3K/Akt pathway in resistance to drug-induced apopotosis. They showed that the inhibitior LY2940002 which inhibits PI3K sensitized breast cancer cell lines to cerulenin induced apopotosis. Nuutinen, U. et al., Exp Cell Res., 312:322-30 (2006) reported recently that inhibition of PI3K or Akt markedly enhanced dexamethasome induced apoptosis in a human follicular lymphoma cell line. These findings are consistent with our present findings demonstrating that treatment of Ramos with LY294002 sensitizes cells to both CDDP and ADR-induced apoptosis. In addition, we demonstrate that the treatment of rituximab-resistant RR1 cells, which cannot be chemosensitized by rituximab, can be sensitized by LY294002 to drug-induced apoptosis. This finding is of potential therapeutic application in the treatment of drug and rituximab-resistant tumor cells. In addition to LY294002, knockdown of Akt by siRNA significantly reduced Bcl-_{XL} expression as well as both AKT and p-Akt and the cells were sensitized to CDDP-induced apoptosis. Clinically, knockdown of Akt by anti-sense or siRNA significantly reduced tumor cell growth and invasiveness and induced cell growth arrest and apoptosis in tumor cells overexpressing Akt (Cheng, J. et al., Proc Natl Acad. Sci USA, 93:3636-41 (1996); Remy, I. et al., Mol Cell Biol., 24:1493-504 (2004); Tabellini, G. et al., J Cell Physiol., 202:623-34 (2005)).

Bcl-_{XL}, a member of the Bcl-2 family, exerts an anti-apoptotic effect on lymphocytes. Zhao, W. et al., Blood, 103:695-7 (2004) investigated the clinical significance of Bcl-_{XL} expression in patients with follicular lymphoma using real time quantitative RT-PCR. Lymph node sections and laser micro-dissected lymphoma cells from 27 patients were analyzed. The gene was overexpressed in patients with follicular lymphoma at a higher level in micro-disected lymphoma cells. A high Bcl-_{XL} level was significantly associated with progression and with the international prognostic index indicating high risk. Moreover, Bcl-_{XL} gene overexpression was linked to short overall survival (Zhao, W. et al., Blood, 103:695-7 (2004)). Bcl-_{XL} is abundantly expressed in lymphoma (Xerri, L. et al., Br J Haematol., 92:900-6 (1996)) and protects the cells from apoptosis induced by DNA damaging agents. An inverse correlation was found between levels of Bcl-_{XL} and sensitivity to 122 standard cancer agents has been established (Amundson, S. et al., Cancer Res., 60:6101-10 (2002)). In the present study, there was a strong correlation between Bcl-_{XL} inhibition by rituximab treatment and chemosensitization.

Various mechanisms contribute to activation of Akt pathways in tumors, including perturbation of upstream PTEN and PIP3 (Vivanco, I. et al., Nat Rev Cancer, 2:489-501 (2002)). Others include autocrine or paracrine stimulation of receptor tyrosine kinases and overexpression of growth factor receptors and/or Ras activation. It has been shown that Akt is activated constitutively by active Ras and Src (Datta, S. et al., Genes & Development, 13:2905-2927 (1999); Lin, R. et al., J Biol Chem., 272:31196-202 (1997)). Thus, in Ramos cells, we have reported that rituximab inhibits the Src kinase pLyn and it is possible that this inhibition is involved in the downstream inhibition of the Akt pathway by rituximab. The PTEN tumor suppressor is a negative regulator of the Akt pathway. The loss of PTEN function leads to an elevated concentration of the PIP3 substrate and results in constitutive activation of downstream components of the PI3K pathway, including the Akt and mTOR kinases (Di Cristofano, A. et al., Cell, 100:387-90(2000)). PTEN is a prominent member and a mechanism leading to selective Akt inhibition. PTEN, as a result of its decreased activity, leads to selective Akt activation. Targeting of Akt, directly or indirectly, inhibits cell proliferation, promote apoptosis, and/or increase sensitivity to chemotherapy (Tachiiri, S. et al., Jpn J Cancer Res., 91:1314-8 (2000)). The loss of PTEN expression has been reported in a variety of cancer including lung, breast, prostate, colon, indometrium, and glioblastoma and has been shown to incur through mutation, deletion, and epigenetic mechanisms (Rennie, P. et al., Cancer Metastasis Rev., 17:401-9 (1998); Marsit, C. et al., Hum Pathol., 36:768-76 (2005); Haiman, C. et al., Cancer Epidemiol Biomarkers Prev., 15:1021-5 (2006)). There are relatively a few studies exploring PTEN abnormalities in lymphoma. Preliminary findings indicate that rituximab treatment of Ramos resulted in PTEN induction.

### Example 3D

### Exemplary Targets for Therapeutic Intervention

The present findings demonstrate that rituximab inhibits the constitutively activated Akt pathway in the Ramos B-NHL cells and results in inhibition of both Bcl-_{XL} activity and expression leading to reversal of drug-resistance. A schematic diagram summarizing the findings of this study is illustrated in FIG. 5. Our findings indicated that modulation of the PI3K/Akt pathway and components of the Akt signal transduction pathway by rituximab are responsible, in part, for chemosensitization. Various gene products of this pathway are potential targets for therapeutic intervention particularly that Akt signaling promotes cell survival, proliferation and invasion. Blocking this pathway could impede the proliferation of tumor cells by increasing sensitivity of tumor cells to undergo apoptosis in response to other cytotoxic agents. Several inhibitors have been the subject of several investigations to intervene in the Akt pathway for tumor cell sensitization (Cheng, J. et al., Oncogene, 24:7482-92 (2005)). Likewise, this study identifies the Akt pathway and each of its component members as set forth on FIG. 5 as targets for therapeutic intervention in the treatment of drug-resistant and/or rituximab-resistant NHL. Hyper-activation of the Akt pathway is thus implicated in the pathogenesis of NHL and the development of drug/rituximab-resistance. Accordingly, as hyper-activation of this pathway may serve as a quite useful prognostic indicator in patients who are refractory to conventional therapies, methods of monitoring such via the activity, activation, expression, or cellular levels of any one or more of the components of the pathways of FIG. 5 can be useful as prognostic indicator.

### Example 4

### Sensitization of Rituximab-sensitive and Rituximab-resistant B-NHL Cell Lines/Clones to TRAIL-induced Apoptosis by Bortezomib and NFκB Inhibitors

Patients with B-NHL respond initially to conventional chemotherapy and/or to immunotherapy with rituximab (alone or in combination with chemotherapy). However, patients develop resistance to these modalities and novel approaches are needed. TRAIL is a cytotoxic molecule that exerts selective anti-tumor cytotoxic activity with minimal toxicity to normal tissues. Further, TRAIL or agonist monoclonal antibody (mAb) to TRAIL receptors, DR4 and DR5, are currently being tested clinically. The present study investigated the sensitivity of B-NHL cell lines to TRAIL-mediated apoptosis using the AIDS-related NHL (ARL) B-cell line, 2F7, and the B-NHL cell lines, Ramos and Daudi. Also, to recapitulate various aspects of acquired rituximab-resistance, we have generated rituximab-resistant (RR) clones from the parental wild type (wt) cells. Rituximab failed to chemo-sensitize the RR clones and the clones exhibited higher resistance to various drugs (e.g., CDDP, VP-16, ADR, Vincristine, Taxol) and to TRAIL (1-250 ng/ml-18 h) compared to the wt cells as analyzed by DNA fragment on assay. The findings demonstrate that the wild type and RR1 cells were resistant to TRAIL-mediated apoptosis at a wide range of TRAIL concentrations. We then examined means to reverse BκTRAIL resistance. We and others have reported that inhibition of NFκB activity can sensitize TRAIL-resistant tumor cells to TRAIL-induced apoptosis. Hence, we examined the effect of the proteasome and NFκB inhibitor, Bortezomib (Velcade), Bay 11-7085 and the specific NFκB inhibitor DHMEQ (Kikuchi et. al, Cancer Research, 63:107 (2003)). Pretreatment of the NHL tumor cells with Bortezomib, Bay 11-7085 or DHMEQ for 2 h followed by treatment with TRAIL for 18 h resulted in significant augmentation of apoptosis and synergy was achieved. Both the rituximab-sensitive and rituximab-resistant tumor cells were sensitized by these inhibitors, though higher concentrations were required for sensitization of the RR clones. Interestingly, detailed analysis of the signaling pathways in the RR clones revealed constitutive hyper-activation of the NFκB survival pathway leading to over-expression of anti-apoptotic gene products Bcl-2, Bcl-_{XL} and Mcl-1. Based on the findings, patients with resistant B-NHL can be treated with combination of TRAIL/anti-DR4 or DR5 mAb and NFκB inhibitors. Alternatively, these patients can be treated with agents that up-regulate TRAIL expression on host effectors (e.g., T cells, NK cells) in combination with NFκB inhibitors.

### Example 5

### Reversal of Rituximab-resistant AIDS-B-NHL Clone to Chemotherapeutic Drug-induced Apoptosis by Bortezomib and DHMEQ

The mechanisms underlying the failure of B-NHL cancer patients to respond to treatment with rituximab, alone or in combination with chemotherapy, are not known. In efforts to address this issue, we have generated rituximab-resistant clones of the AIDS NHL cell line, (2F7RR). Recent findings have demonstrated that treatment of the wild type (wt) 2F7 with rituximab sensitized the tumor cells to various chemotherapeutic drug-induced apoptosis. Chemosensitization was the result of rituximab-mediated inhibition of the p38 MAPK signaling pathway and the selective inhibition of the anti-apoptotic Bcl-2 gene product (Vega et. al., Oncogene, 23:4993 (2004)). Analysis of one clone, 2F7RR1, revealed that the cells have diminished surface CD20 expression and failed to respond to CDC and to apoptosis following cross-linking. In addition, the cells were resistant to rituximab-mediated chemosensitization. In contrast to wt2F7, molecular analysis of the 2F7RR1 clone revealed that rituximab failed to inhibit p-Lyn, p38-MAPK, Bcl_{XL}, and Bcl-2. In addition, rituximab failed to inhibit the transcription factors NF-κB, YY1, SP-1, and STAT3. Noteworthy, 2F7RR1 exhibited higher resistance to drug-induced apoptosis compared to wt2F7 and showed overexpression of Bcl-2. Previous findings with the wt2F7 demonstrated that Bcl-2 was responsible for chemoresistance. Accordingly, we examined whether inhibition of Bcl-2 in 2F7RR1 can reverse chemoresistance. Since Bcl-2 is under the transcriptional regulation of NFκB, we examined the effect of the NF-κB inhibitors Bortezomib and DHMEQ (Kikuchi, et al., Cancer Research, 63:107 (2003)). The findings revealed that treatment of 2F7RR1 with these inhibitors resulted in the reversal of resistance to a number of chemotherapeutic drugs (examples: taxol, vincristine, ADR, CDDP, VP16, etc.). The extent of chemo-sensitization by Bortezomib and DHMEQ was comparable. These studies present evidence that rituximab and drug-resistant tumor cells may be sensitized to chemotherapeutic drug-induced apoptosis via inhibition of NFκB or Bcl-2. These findings also indicate that Bortezomib and DHMEQ can be used to treat rituximab and drug-resistant AIDS-B-NHL and other cancers.

### Example 6

### New Targets Identified for Therapeutic Intervention in the Reversal of Rituximab and Drug Resistant AIDS-B-NHL

Rituximab has been used in the treatment of B Non-Hodgkin's Lymphoma (NHL) with significant clinical responses. However, a subset of patients fails to respond to treatment with rituximab used alone or in combination with chemotherapy. The mechanism by which B-NHL patients resist treatment is not known. We have reported that treatment of the AIDS B-NHL cell line 2F7 with rituximab resulted in significant inhibition of the p38 MAPK pathway and inhibition of Bcl-2 expression concomitantly with chemoresistance. The pivotal role of Bcl-2 in chemoresistance was demonstrated by various methods as inhibition of Bcl-2 expression or activity which sensitized the tumor cells to drug-induced apoptosis (Vega et al., Oncogene, 23(20):3530-40 (2005)). In order to examine the mechanism of NHL resistance to rituximab, we have developed in the laboratory rituximab-resistant clones of 2F7 (2F7RR) and have compared their response with the wild type to rituximab treatment alone and in combination with chemotherapeutic drugs. Unlike the wild type 2F7, rituximab treatment failed to sensitize 2F7 RR1 to drug-induced apoptosis, failed to modulate the p38MAPK/NF-κB/YY1/STAT3 signaling pathways, did not inhibit Bcl-2 expression. We examined the effect of various chemical inhibitors of this signaling pathway on chemosensitization. We demonstrate that treatment with the proteasome inhibitor Bortezomib or the NF-κB inhibitor DHMEQ significantly sensitized 2F7-RR1 cells to drug (CDDP, vincristine, adriamycin, VP 16, taxol)-induced apoptosis. These inhibitors also resulted in the inhibition of NF-κB, YY1 and downregulated Bcl-2 expression. These findings demonstrate that rituximab and drug-resistant clone 2F7-RR1 can be sensitized to reverse chemoresistance. The findings also identify intracellular targets whose modification can reverse resistance. Such targets include the p38 MAPK pathway, the transcription factors NF-κB, YY1, or STAT3 and also inhibitors of Bcl-2 expression and/or activity. These findings also suggest that combination treatment of currently used drugs such as Bortezomib and chemotherapeutic drugs have a potential for the treatment of rituximab and drug-resistant AIDS-B-NHL.

### Example 7

### Rituximab-mediated Inhibition of the Akt Pathway and Upregulation of PTEN Expression in Ramos B-NHL Leading to Chemosensitization to Drug-induced Apoptosis

Rituximab treatment of drug resistant B-NHL cell lines sensitizes the tumor cells to drug (e.g. CDDP, VP16, Taxol, Vincristine, Adriamycin)-induced apoptosis. The PI3K/Akt signaling pathway has been shown to be involved in cell proliferation and negatively regulates apoptosis-inducing stimuli. Hence, we examined if rituximab affects the PI3K/Akt pathway as a mechanism of chemosensitization of the tumor cells. Ramos B-NHL cells were treated with predetermined optimal concentration of rituximab (20 ug/ml) for different periods of times (3-20 h) and the cells were harvested and total cell lysates were prepared. Analysis of lysates by Western revealed that treatment with rituximab inhibits the constitutively activated phospho-PI3K, phospho-Akt, phospho-Bad, (no inhibition of non-phospho related proteins), and downregulated Bcl-_{XL} expression as early as 6 h post treatment. Bad and form complexes and following phosphorylation of Bad by the Akt pathway, Bcl-_{XL} dissociates from the complex and participates in the regulation of resistance to apoptosis. Rituximab treatment of Ramos cells was shown to inhibit phospho-Bad and there was significant enhancement of the Bcl-_{XL} complexed with Bad, thus reducing free Bcl-_{XL} and contributing to chemosensitization-mediated by rituximab. It has been reported that the constitutive activation of Akt is regulated in large part by PTEN activity. PTEN is a tumor suppressor that serves as a major negative regulator of the survival signaling mediated by the PI3K/Akt/protein kinase B pathways. The constitutive activation of Akt in Ramos was paralleled by low PTEN levels expression in the cells. Treatment of Ramos with rituximab resulted in a significant induction of PTEN expression in the cells. Time kinetic analysis revealed that rituximab augments PTEN expression as soon as 9 h after treatment (suggesting a post Akt-induced inhibitor). We then examined the role of the PI3K/Akt pathway in the regulation of Ramos resistance to drugs. Treatment of the Ramos cells with the specific Akt inhibitor LY294002 sensitized the cells to CDDP-induced apoptosis concomitant with downregulation of Bcl-_{XL} expression. These findings demonstrate for the first time that rituximab treatment can inhibit the constitutive Akt pathway in B-NHL cells and in addition results in the induction of the expression of the tumor suppressor PTEN. These findings also reveal different targets whose modulation can reverse the resistance of tumor cells to drug-induced apoptosis.

### Example 8

### Characteristics of Rituximab-resistant B-NHL Clones: Deficiencies in Rituximab-mediated Changes in Lipid Raft Microdomains and Cell Signaling

The chimeric mouse anti-human CD20 mAb Rituximab, alone or combined with chemotherapy, has significant anti-lymphoma activity. A subset of patients does not respond initially or following treatment with rituximab. In order to investigate the mechanism of rituximab resistance, we have developed rituximab-resistant (RR) clones of Ramos and Daudi cells following culture with increasing concentrations of rituximab and limiting dilution analysis of single cells. The CD20+ expressing RR clones failed to respond to rituximab-mediated CDC, cytostasis, chemo-sensitization, cross-linked rituximab-mediated apoptosis (Jazirehi and Bonavida, Oncogene, 24:2121-43 (2005)). Studies were undertaken to examine the underlying mechanism of rituximab resistance in the clones. The RR clones showed over-expression of the complement inhibitor CD59. In the wild type cells, treatment with rituximab resulted in a rapid and transient increase in acid-sphingomyelinase activity concomitant with cellular ceramide generation in lipid rafts. Rituximab treated cells externalized both acid sphingomyelinase and ceramide which co-localized with the CD20 receptor (Bezombes et al., Blood, 104:1166-73 (2004)). Preliminary results show that rituximab-induced acid sphingomyelinase translocation and ceramide generation at the cell surface is reduced in RR clones compared to parental clones. We further analyzed possible mutations in the CD20 coding sequence. There was no difference in mutations between the parental and RR clones. Since lipid rafts serve as signaling platforms, thus, it seems that resistance to rituximab in these clones is due to a defect of the sphingomyelin-ceramide pathway. RR clones exhibit hyperactivation of the ERK1/2 and NF-kB survival signaling pathways concomitant with overexpression of Bcl-_{XL}, Bcl-2, and Mcl-1. Treatment of RR clones with specific pharmacological inhibitors of these pathways sensitized the RR clones to various chemotherapeutic drugs (e.g., CDDP, Vincristine, Adrimycin, Taxol, Etoposide) and significant synergy in apoptosis was achieved. These findings demonstrate that the development of resistance to rituximab treatment may result from alteration of the cell signaling mediated by rituximab and failure to mobilize the lipid rafts on the cell membrane and the failure to inhibit downstream survival signaling pathways. In addition, the findings suggest the potential therapeutic application of combining sensitizing agents and conventional therapeutic drugs in the treatment of rituximab and drug-resistant B-NHL.

### Example 9

### Development of Rituximab-resistant Lymphoma Clones with Altered Cell Signaling and Cross-resistance to Chemotherapy: Circumvention of Acquired Resistance by Specific Pharmacological Inhibitors (Bortezomib, DHMEQ, PD098059

The B-cell specific surface marker CD20 (Tedder, T. et al., Immunol. Today, 15:450-4 (1994)) does not circulate in the plasma as a free protein, which could potentially block Ab binding to the cells (Andeson, K. et al., Blood, 63:1424-33 (1984)). Also, it is neither internalized upon Ab ligation (Press, 0. et al., Blood, 69:584-91 (1987)) nor shed from cell surface (Einfeld, D. et al., EMBO J, 7:711-7 (1988)); properties that make CD20 an ideal target for immunotherapy of NHL. The chimeric mouse anti-human CD20 mAb rituximab (IgG1κ) binds with high affinity to CD20 expressing cells. It is the first FDA approved mAb for NHL treatment (Grillo-Lopez, A., Int J Hematol., 76:385-93 (2002)).

Rituximab has been an important addition to the therapeutic armamentarium against low-grade follicular NHL (Dillman, R., Semin Oncol., 30:434-47 (2003)). Furthermore, its utilization alone or combined with chemotherapy is considered as first line therapeutic option for other types of hematological malignancies (Lin, T. et al., Semin Oncol., 30:483-92 (2003); Hiddemann, W. et al., Semin Oncol., 30:16-20 (2003); Ghobrial, I. et al., Lancet Oncol., 4:679-85 (2003)) improving patients' survival. Its usage is also extended to other pathologic states culminating in long-lasting response (Risken, N. et al., Neth J Med., 61:262-5 (1993); Pels, H. et al., Onkologie, 26:351-4; Reams, B. et al., Chest, 124:1242-9 (2003); Weichert, Z. et al., J Cutan Med. Surg., 7:460-3 (2003)). Rituximab exerts significant anti-tumor activity in vivo (Reff, M. et al., Blood, 83:435-5 (1994)) via inhibition of cell proliferation or triggering multiple cell-damaging mechanisms including antibody-dependent cellular cytotoxicity (ADCC), complement-dependent cytotoxicity (CDC) and apoptosis (Maloney, D., J Clin Oncol., 23:6421-8 (2005); Smith, M., Oncogene, 22:7359-68 (2003)). It also augments the cytotoxic effects of drugs on drug-resistant NHL B-cells (Jazirehi, A. et al., Oncogene, 24:2121-43 (2005)). Nonetheless, the contribution of these mechanisms on primary normal and malignant B-cells in vivo and the molecular mechanisms of rituximab action need to be defined.

We have explored the effects of modifications of signaling pathways by rituximab on its chemo-sensitizing attributes. To this end we have recently reported that rituximab, via inhibition of NF-κB and ERK1/2MAPK pathways, reduces Bcl-_{XL} expression and chemo-sensitizes NHL B-cells (Jazirehi, A. et al., Cancer Research, 64:7117-26 (2004); Jazirehi, A. et al., Cancer Research, 65:264-76 (2005)). Activation of NF-κB and ERK1/2 pathways are emerging as major mechanisms of tumor cell drug-resistance and induce their rapid proliferation. Thus, interruption of these pathways is a target for therapeutic intervention and may confer drug-sensitivity (Ghosh, S. et al., Cell, 109:S81-S96 (2002); Dent, P. et al., Clin Cancer Res, 7:775-83 (2001)), which has proven successful in enhancing the apoptotic effects of TNF-α and CPT-11 resulting in tumor regression in vivo (Wang, C. et al., Nature Medicine, 5:412-7 (1999)). Inhibition of NF-κB and ERK1/2 pathways was shown by decrease in phosphorylation and kinase activities of the signaling molecules and reduced NF-κB and AP-1 DNA-binding ability (DBA) concomitant with reduction in the expression of their common down-stream target (24) Bcl-_{XL}.

The superior efficacy of CHOP+rituximab (R-CHOP) compared to CHOP (cyclophosphamide, doxorubicin, vincristine and prednisone) alone in elderly DLBCL patients was reported, where the combination therapy resulted in higher rates of complete remission and survival (Coiffier, B. et al., Anticancer Drugs, 2:S43-50 (2002)) and also increases overall survival in aggressive Bcl-2-positive and -negative patients (Mounier, N. et al., Haematologica, 91:715-6 (2006)). Despite its well-established clinical efficacy, a sub-population of patients, via an elusive mechanism, does not respond to rituximab and/or acquires resistance upon long-term rituximab therapy (Maloney, D., J Clin Oncol., 23:6421-8 (2005); Smith, M., Oncogene, 22:7359-68 (2003)). Based on previous reports we hypothesized that development of rituximab-resistance may be related to tumor cells' failure to respond to rituximab-mediated signaling. Further, the unresponsiveness of the cells to drug therapy (alone or combined with rituximab) may be due to hyper-activation of survival signaling pathways and up-regulation of resistant-factors. Due to challenges in obtaining patient-derived specimens for analysis, and to recapitulate various aspects of acquired rituximab-resistant situations, rituximab-refractory (RR) clones were generated (Jazirehi, A. et al., Blood, 104:3410 (2004))*. Using a battery of functional and biochemical assays, representative clones were compared to parental cells to examine alterations in rituximab-mediated effects to examine the above hypotheses. Different RR clones have also been established and analyzed (Olejniczak, S. et al., Blood, 106:4819 (2005))**. The following objectives were investigated: 1) phenotypic and functional properties of RR clones (e.g., differences regarding CD20 surface expression, proliferation, CDC, cross-linked rituximab-mediated apoptosis), 2) chemo-sensitivity of the clones and chemo-sensitization by rituximab, 3) activation status of ERK1/2 and NF-κB pathways, 4) expression of Bcl-2 family members, and 5) effects of various inhibitors of survival pathways on reversal of chemo-resistance.

Materials and Methods

Cell Lines and Clones.

C.D20⁺ human Burkitt's lymphoma B-cell lines Daudi and Ramos were obtained from ATCC (Bethesda, Md.). For the generation of RR clones wt cells were grown in the presence of step-wise increasing concentrations of rituximab (5-20 μg/ml-10 weeks). Single cells were then subjected to three consecutive rounds of limiting dilution analysis (LDA) (Jazirehi, A. et al., Blood, 104:3410 (2004)). Single cells were propagated and maintained in RPMI-1640 medium supplemented with 10% (v/v) heat-inactivated fetal bovine serum (FBS) (Jazirehi, A. et al., Mol. Cancer Therapeutics, 2:1183-93 (2003)). Clones were supplemented with rituximab (20 μg/ml) once a week and grown in rituximab-free medium at least one week prior to analysis. Cultures were incubated in controlled atmosphere incubator at 37° C. with saturated humidity at 0.5×10⁶ cells/ml.

Reagents.

Paclitaxel, CDDP, VP-16, ADR, and vincristine were purchased from Sigma (St. Louis, Mo.) and were diluted in DMSO. DMSO concentration did not exceed 0.1 % in any experiment. Mouse anti-Bcl-_{XL}, -Mcl-1,-Bcl-2 mAbs were purchased from Santa Cruz Biotechnology (Santa Cruz, Calif.) and DAKO (Carpinteria, Calif.), respectively. Mouse anti-p-IκB-α (Ser^{32/36}), -actin mAbs were obtained from Imgenex (San Diego, Calif.) and Chemicon (Temeculla, Calif.), respectively. Rabbit p-IKKα/β [Ser^{180/181}] Ab was obtained from Cell Signaling (Beverley, Mass.). Rabbit anti-p-ERK1/2 (Thr¹⁸⁵/Tyr¹⁸⁷) Ab, MAPK kinase substrate-4 (aa 172-192) and PD098059 were obtained from Biosource (Camarillo, Calif.). 2MAM-A3 was purchased from Biomol (Plymouth, Pa.). DHMEQ was provided by Dr. K. Umezawa (Tokyo, Japan). Rituximab and bortezomib were procured commercially.

Surface CD20 Expression.

Cells (2×10⁶) were washed twice with ice cold 1×PBS and stained with 1 □g mouse anti-human CD20 mAb (IDEC-2B8; IDEC Pharmaceuticals, San Diego, Calif.) or isotype control (pure IgG1) (20 minutes on ice, light protected). Then, the cells were washed twice with ice cold 1×PBS, stained with FITC-labeled secondary Ab (30 minutes on ice, light protected) and subjected to FACS analysis.

Immunoblot Analysis.

Cells (10⁷) were either grown in complete medium or complete medium supplemented with various inhibitors. Cells were lysed at 4° C. in radioimmuno-precipitation assay (RIPA) buffer [50 mM Tris-HCl (pH 7.4), 1% NP-40, 0.25% sodium deoxycholate, 150 mM NaCl] supplemented with one tablet of protease inhibitor cocktail (Complete Mini; Roche). A detergent-compatible protein (DC) assay kit (Bio-Rad, Hercules, Calif.) was used to determine protein concentration. An aliquot of total protein lysate was diluted in an equal volume of 2×SDS sample buffer, boiled for 10 min, and cell lysates were electrophoresed on 12% SDS-PAGE gels. Western blot was carried out as described (Jazirehi, A. et al., Mol. Cancer Therapeutics, 2:1183-93 (2003)). The relative intensity of bands, hence, relative alterations in protein expression, was assessed by densitometric analysis of digitized images using public domain NIH image program (http://rsb.info.nih.gov/nih-image/).

Assessment of Apoptosis. A. DNA Fragmentation Assay.

Percentage of apoptotic cells was determined by evaluation of propidium iodide (PI) stained preparations of cells using an Epic_{XL} flow-cytometer. Cellular debris was excluded from analysis by raising the forward scatter threshold, and DNA content of the intact nuclei was recorded on a logarithmic scale (Nicoletti, I. et al., J Immunol. Methods, 139:271-9 (1991)). Percent apoptosis is represented as percentage of hypodiploid cells accumulated at sub-G0 phase of cell cycle.

Evaluation of Active Caspase-3 Levels.

Levels of active caspase-3 were evaluated with FITC-labeled anti-active caspase-3 mAb (PharMingen, San Diego, Calif.) (Jazirehi, A. et al., Mol. Cancer Therapeutics, 2:1183-93 (2003)).

Assessment of Viable Cell Recovery.

This was assessed using standard XTT assay kit (Roche, Indianapolis, Ind.) that measures metabolic activity of viable cells (Scudiero, D. et al., Cancer Res., 48:4827-33 (1998)). Percent cell recovery was calculated using background-corrected reading as follows: % Cell Recovery=[(OD of sample wells/OD of untreated cells)]×100.

Electrophoretic Mobility Shift Analysis.

The DBAs were evaluated using biotin-labeled oligonucleotide AP-1 (5′-CGCTTGATGACTCAGCCGGAA-3′) (Lee, W et al., Cell, 49:741-52 (1987)) and NF-κB (5′-AGTTGAGGGGACTT TCCCAGGC-3′) probes (Harada, H. et al., Mol. Cell Biol., 14:1500-9 (1994)) using EMSA kit (Panomics, Inc., Redwood City, Calif.) according to manufacturer's instructions. 10 μg of nuclear extracts were subjected to denaturing 5% PAGE and developed (Jazirehi, A. et al., Cancer Research, 64:7117-26 (2004); Jazirehi, A. et al., Cancer Research, 65:264-76 (2005)).

Immune-complex Kinase Assay.

The kinase activity of IKK and MEK1/2 was assessed by their ability to phosphorylate IκB-α (Ser^{32/36}) and MAPK kinase substrate 4 (Thr¹⁸⁵/Tyr187) using a slightly modified version of previous methods (Alessi, D. et al., J. Biol. Chem., 270:27489-94 (1995); Jazirehi, A. et al., Cancer Research, 64:7117-26 (2004); Jazirehi, A. et al., Cancer Research, 65:264-76 (2005)).

Quantitative Real-time PCR (qPCR).

Samples were analyzed in triplicate with iQ SYBR Green Supermix using iCycler Sequence Detection System (BioRad). Total RNA was extracted from 10⁷ cells for each condition with 1 ml/sample of STAT-60 reagent and quantified by 3.1.2 NanoDrop ND-1000 spectrophotometer. 3 μg of total RNA was reversed to first-stranded cDNA for 1 h at 42° C. with 200 units SuperScript II RT and 20 μM random hexamer primers. Amplification of 2.5 μl of cDNAs was performed using gene-specific primers. Internal control for equal cDNA loading in each reaction was assessed using G-3-PDH primers. Amplicons were resolved by 2% gels for confirmation and were of expected size. Percentages of expression of each molecule were calculated with the assumption that control samples were considered as 100%.

Statistical Analysis.

Assays were set up in triplicates and results were expressed as mean±standard deviation (STD). Statistical analysis and P values were calculated by two-tailed paired t test with a confidence interval (CI) of 95% for determination of significance of differences between treatment groups. (P<0.05: significant). ANOVA was used to test significance among the groups using InStat 2.01 software.

### Example 9A

### Phenotypic and Functional Properties of the RR Clones

(i) Diminished Surface CD20 Expression and Failure to Respond to Rituximab-Mediated Inhibition of Cell Growth and Apoptosis Following Cross-linking.

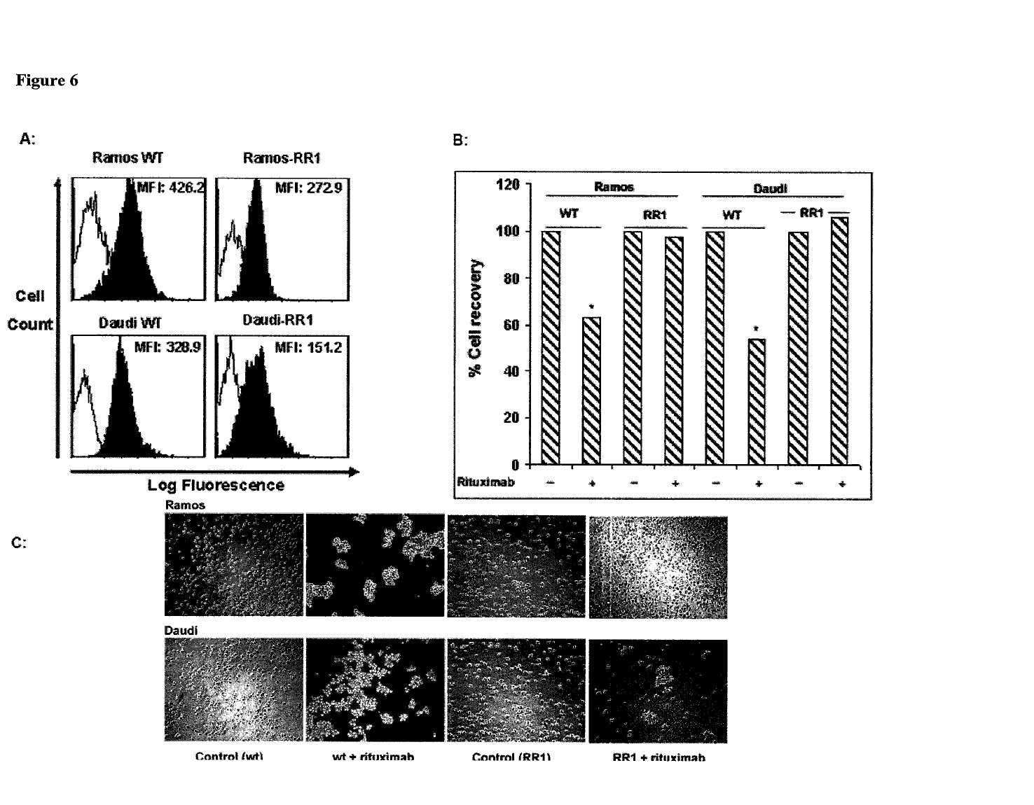

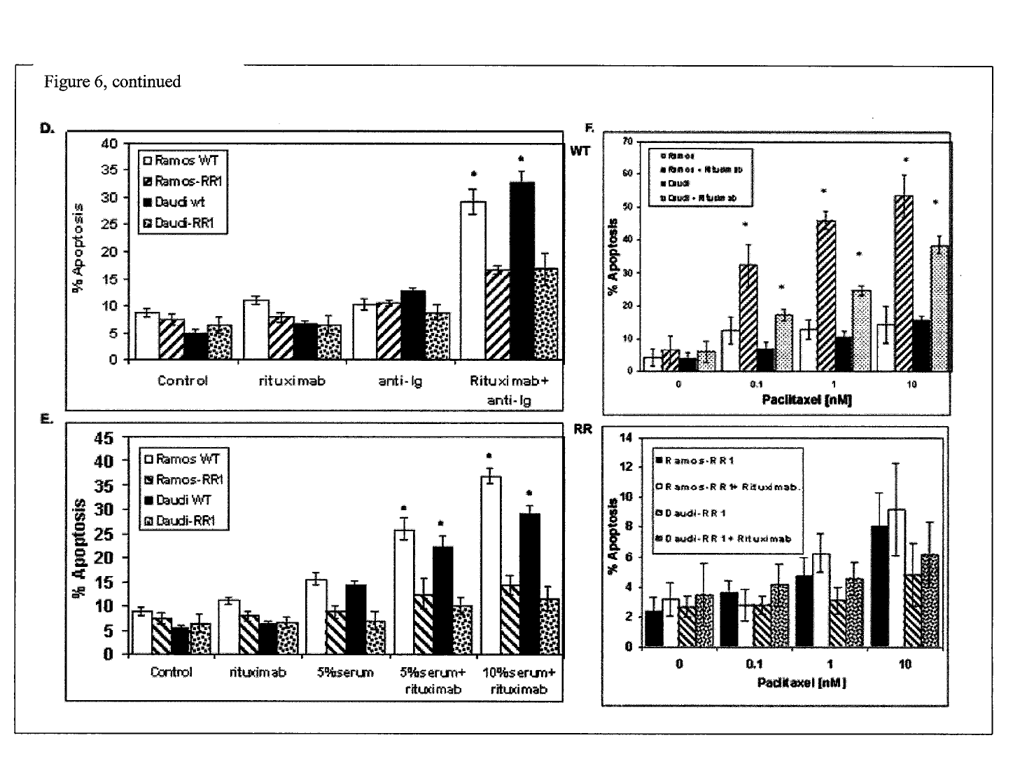

Wild type (wt) cells and Ramos-RR1 and Daudi-RR1 clones were stained with isotype control (pure IgG1; gray lines) or FITC-labeled anti-CD20 mAb (IgG1 subtype; solid black lines) and subjected to FACS analysis. As measured by mean fluorescence intensity (MFI), wt cells show significant CD20 surface expression while the clones exhibit ˜40-50% reduction in surface CD20 (Ramos: 468.4±14.0 vs. 264±9.6, Daudi: 346±11.4 vs. 156.4±10.2) (FIG. 6A). Similar results were obtained in multiple independent experiments with other Ramos-RR and DLBCL-RR clones (data not shown) suggesting that continuous rituximab treatment results in diminished, but not complete loss of, surface CD20 expression on RR clones.

To assess the ability of RR clones to respond to growth inhibitory effects of rituximab, both the wt and the clones were left either untreated or treated with a predetermined concentration of rituximab for wt cells (20 μg/ml-24 h) (34). An aliquot (10⁴) of cells was used in a standard XTT viability assay. Rituximab exerts an inhibitory effect on the wt cells (Ramos: 27%, Daudi: 46%) while it fails to reduce the growth of RR clones (Ramos-RR1: 98%, Daudi-RR1: 106%) (FIG. 6B). Higher concentrations (50, 100 μg/ml) or longer exposure time (up to 48 h) of rituximab did not alter the phenotype of the clones (data not shown); thus, 20 μg/ml rituximab (24 h) was used in subsequent studies. Results of the XTT assay were confirmed by trypan blue dye exclusion and FACS (data not shown), which essentially showed that rituximab fails to induce apoptosis or inhibit cell growth in RR clones. Untreated RR clones exhibited homotypic aggregation that remained unaffected by rituximab. In contrast, wt cells grew as suspended non-aggregated and rituximab caused them to form clumps (FIG. 6C). These results indicate that while rituximab efficiently lowers the growth rate of the wt cells, it fails to reduce the growth of RR clones.

Next, we assessed the ability of cross-linked rituximab to induce apoptosis in RR clones. The wt cells and the clones were pre-treated with optimal concentration of cross-linked rituximab (50 μg/ml anti-human immunoglobulin (hIg)+20 μg/ml rituximab-24 h) (Shan, D. et al., Blood, 91:1644-52 (1998)) and subjected to apoptosis assay. DNA fragmentation assay for apoptosis detection in subsequent experiments was confirmed by measuring active caspase-3 levels (28, data not shown). Neither rituximab (Ramos-RR1: 11.0±0.8%, Daudi-RR1: 6.7±0.6%) nor the anti-hIg (Ramos-RR1: 10.2±1.1%, Daudi-RR1: 12.8±0.5%) alone efficiently killed the cells. However, combination of the two agents (cross-linked rituximab) induced significant levels of apoptosis in Ramos (29.2±2.4%) and Daudi (32.8±2.1%) wt cells, while cross-linked rituximab moderately killed RR clones (FIG. 6D) suggesting that clones have developed higher threshold (Ramos-RR1: 2.86, Daudi-RR1: 2.56 folds) and, unlike the wt cells, do not efficiently respond to cross-linked rituximab-mediated apoptosis.

(ii) Failure to Respond to CDC

The ability of rituximab to mediate CDC in RR clones compared to the wt cells was assessed by analyzing the percentage of dead cells that were treated with human AB serum as source of complement (5, 10%-24 h). As shown in FIG. 6E, wt cells exhibited modest sensitivity to the cytotoxic effects of AB serum (as a function of serum concentration) (Ramos: 15.5±1.3%, Daudi: 14.3±0.9%); an effect that was significantly augmented in the presence of rituximab (Ramos: 36.9±1.5% vs. 11.0±0.8%, Daudi: 29.2±1.6% vs. 6.2±0.6%). There was augmentation of cytotoxicity with 10% serum than with 5%. However, compared to wt cells, the clones were less sensitive to human AB serum, and rituximab failed to enhance their sensitivity (Ramos-RR1: 14.2±1.6% vs. 7.8±0.6%, Daudi-RR1: 11.6±2.4% vs. 6.6±1.1%). Increasing serum concentration (15%) neither enhanced their sensitivity nor augmented rituximab-mediated CDC of the clones (data not shown). These results show that wt cells are sensitive to CDC which is enhanced by both serum levels and rituximab treatment, while long-term rituximab exposure is accompanied by higher CDC-resistance in the clones (Ramos-RR1: 2.6 fold, Daudi-RR1: 2.5 folds) and rituximab fails to augment CDC.

(iii) Failure of Rituximab to Chemo-sensitize the RR Clones

Augmentation of the cytotoxic effects of drugs is an established property of rituximab (Jazirehi, A. et al., Oncogene, 24:2121-43 (2005)). To assess the ability of rituximab to chemo-sensitize RR clones, wt cells and the clones were pretreated with rituximab, subsequently treated with various concentrations of paclitaxel (0.1-10 nM) and subjected to apoptosis assay. Paclitaxel was used as a representative drug; similar results were obtained with etoposide (VP-1 6) and cis-platinum (CDDP) (data not shown). Rituximab significantly augmented the apoptotic effect of paclitaxel in wt Ramos and Daudi cells in a concentration-dependent manner (range 45-58% apoptosis) (FIG. 6F). However, RR clones had higher resistance to paclitaxel, and rituximab was incapable of augmenting paclitaxel-induced apoptosis (FIG. 6F). 2.5-5 folds higher concentrations of rituximab failed to sensitize the clones (data not shown). These results suggest that while rituximab efficiently chemo-sensitizes wt cells it is incapable of chemo-sensitizing the clones suggestive of higher resistance of RR clones to rituximab-mediated chemo-sensitization.

### Example 9B

### Development of Higher Drug-resistance in RR Clones

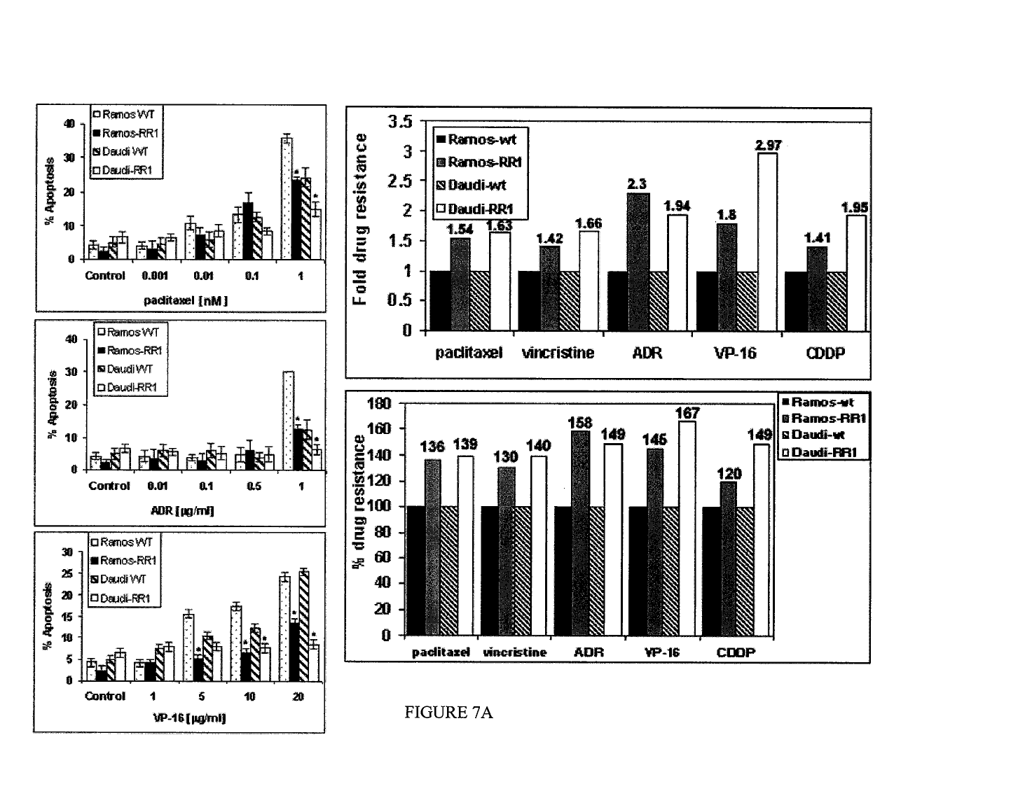

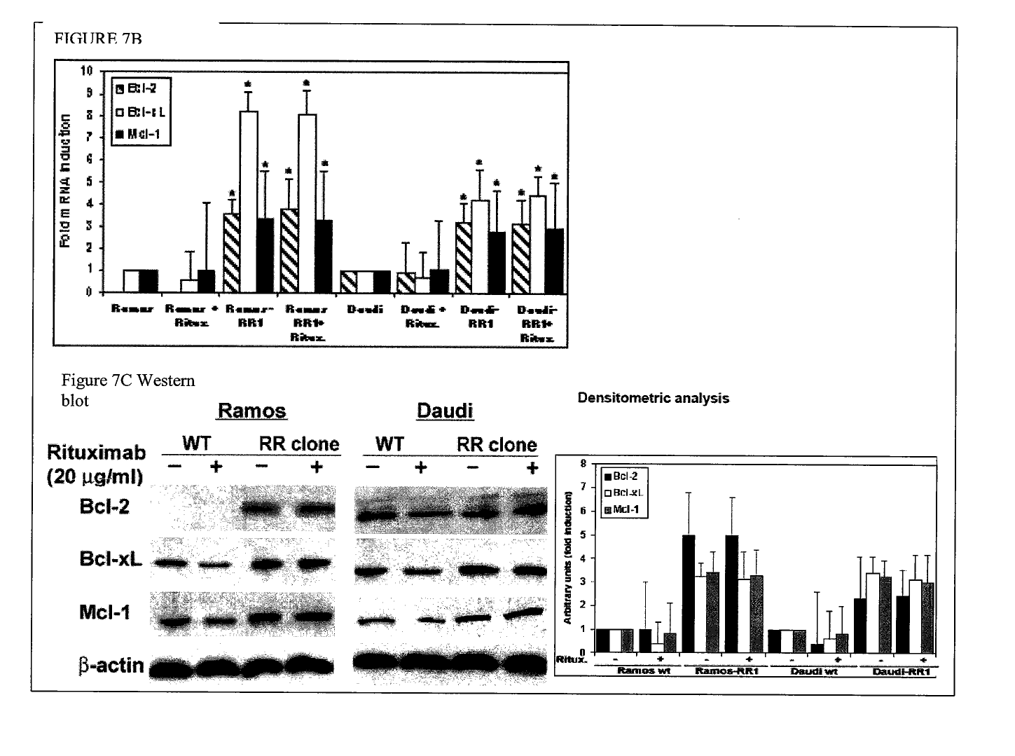

Since RR clones were not chemo-sensitized by rituximab, their sensitivity against a battery of drugs including paclitaxel, vincristine, VP-16, adriamycin (ADR), and CDDP was examined. Compared to wt cells, which exhibit moderate sensitivity to these drugs in a concentration-dependent manner, clones exhibited higher apoptosis threshold to these drugs, albeit to varying degrees. As such, Ramos-RR1 showed 1.54 (136%), 1.42 (130%), 2.3 (158%), 1.8 (145%), 1.41 (120%) folds and Daudi-RR1 showed 1.63 (139%), 1.66 (140%), 1.94 (149%), 2.97 (167%), 1.95 (149%) folds resistance to paclitaxel, vincristine, ADR, VP-16, and CDDP, respectively, compared with their respective wt cells (FIG. 7A). Prolonged incubation time (48 h) did not significantly augment drug cytotoxic in the clones (data not shown) suggesting that compared to wt cells, RR clones exhibit higher (1.41-2.97 fold) drug-resistance. Functional analysis of the multi-drug resistance (MDR) pump showed the existence of functional MDR pump in both wt cells and the clones. Further, RR clones exhibited no functional impairment of MDR pump (±rituximab, data not shown) suggesting that higher drug-resistance in the clones is independent of the MDR pump.

### Example 9C

### Over-expression of Bcl-2, Bcl-_{XL} and Mcl-1 in RR clones.

RR clones did not respond to rituximab-mediated chemo-sensitization and exhibited higher drug-resistance. Previous findings have established Bcl-_{XL} as an important resistant-factor (Jazirehi, A. et al., Cancer Research, 64:7117-26 (2004); Jazirehi, A. et al., Cancer Research, 65:264-76 (2005)); thus, we evaluated Bcl-_{XL} levels and other anti-apoptotic Bcl-2 family members in the clones. Total RNA was extracted and converted to first stranded cDNA which was subjected to real-time quantitative-PCR (qPCR) analysis. RR clones exhibited increased expression of Bcl-2 (Ramos-RR1: 3.6, Daudi-RR1: 3.2 fold), Bcl-_{XL} (Ramos-RR1: 8.2, Daudi-RR1: 4.2 fold), Mcl-1 (Ramos-RR1: 3.4, Daudi-RR1: 2.8 fold) at the transcription level (FIG. 7B). Immunoblot showed that clones exhibit higher Bcl-2, Bcl-_{XL}, Mcl-1 protein levels (˜2.3-5.0 fold) compared to wt cells. Interestingly, expression levels of these proteins remained unaffected by rituximab treatment of the clones, while Bcl-_{XL} levels in wt cells were reduced (FIG. 7C). Notably, expression levels of other pro- and anti-apoptotic factors (Bcl-_{xS}, Bfl-1, /A1, Bad, Bax, Bid, Bak, c-IAP-1,-2, survivin, XIAP) were similar in wt cells and RR clones (±rituximab) (data not shown). These results show that clones express higher levels of protective factors which may explain their unresponsiveness to rituximab-mediated chemo-sensitization and higher drug-resistance. Also rituximab was unable to reduce the levels of resistant-factors suggesting that signaling pathways in the clones are no longer responsive to rituximab.

### Example 9D

### Hyper-activation of the ERK1/2 and NF-□B Signaling Pathways in the RR Clones

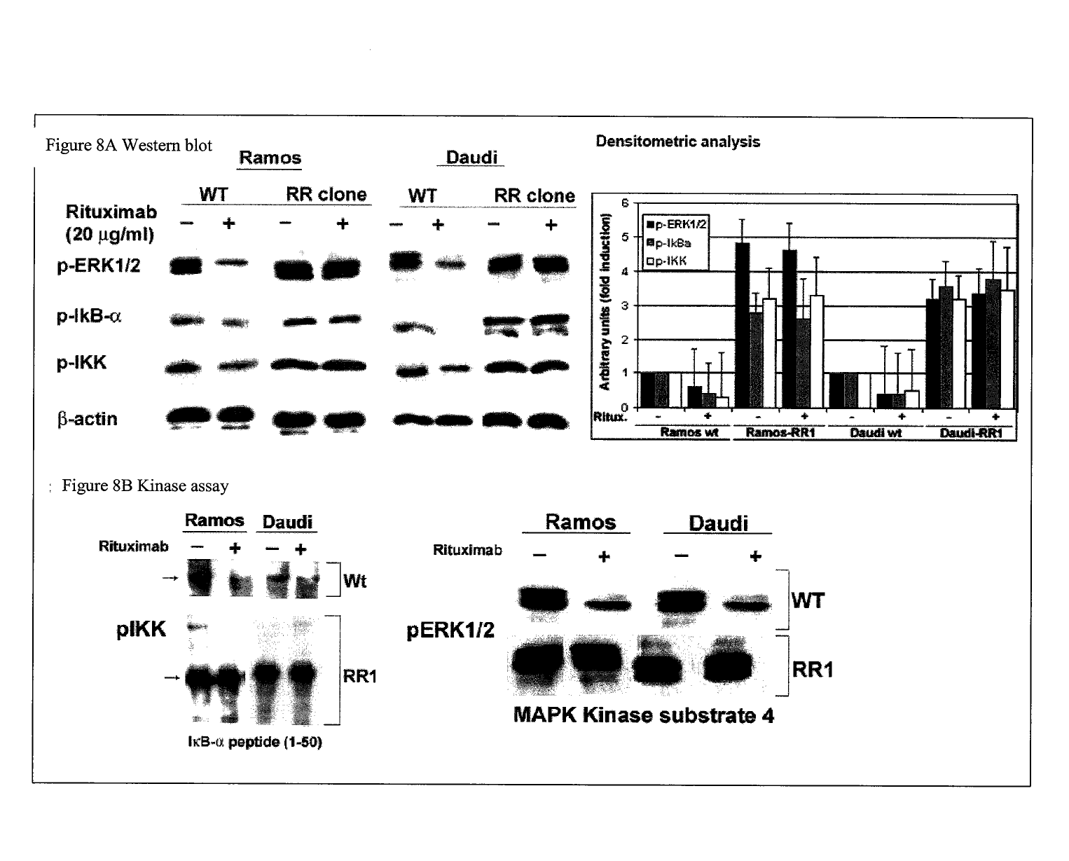

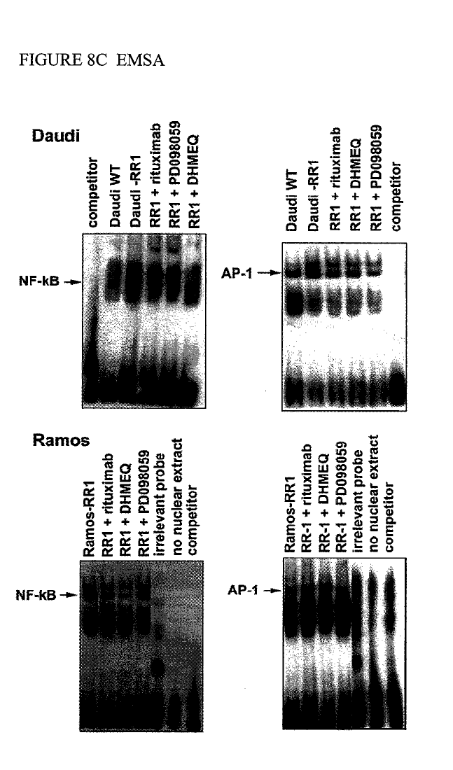

The above findings suggest that the dynamics of the cellular signaling pathways are altered in the clones. Rituximab inhibits ERK1/2 and NF-κB pathways leading to reduced DBA of AP-1 and NF-κB transcription factors in wt cells (Jazirehi, A. et al., Cancer Research, 64:7117-26 (2004); Jazirehi, A. et al., Cancer Research, 65:264-76 (2005)). Thus, we examined the activation status of these pathways in the clones. Whole cell extracts of wt cells and the clones (±rituximab) were subjected to immunoblot for components of NF-κB and ERK1/2 pathways. The phosphorylation-dependent state of IKK, IκB-α and ERK1/2 was higher in the clones (˜3.2-4.8 folds) than wt cells. Basal levels of these signaling molecules remained unaffected in wt and RR clones (±rituximab, data not shown). Rituximab significantly reduces the phosphorylation of these molecules in wt cells (Jazirehi, A. et al., Cancer Research, 64:7117-26 (2004); Jazirehi, A. et al., Cancer Research, 65:264-76 (2005)), an effect that is not observed in clones (FIG. 8A) suggesting that molecular switches responsible for rituximab-mediated de-phosphorylation of these molecules are no longer operative in clones.

To ascertain the observed hyper-phosphorylation results in increased activity of NF-κB and ERK1/2 pathways, immune complex kinase assays were performed to assess IKK and MEK1/2 kinase activities of the clones (±rituximab) using IκB-α peptide (IKK substrate) and MAPK kinase substrate-4 (ERK1/2 substrate). Untreated clones showed significantly increased kinase activities as shown by increased ability of the lysates to phosphorylate their specific substrates, whereby rituximab did not reduce the IKK and MEK1/2 kinase activities. This phenomenon was not observed by IκB-α peptide S32/36A (data not shown). In contrast, in wt cells rituximab diminishes the kinase activity of IKK and MEK1/2 (FIG. 8B), thus, increased phosphorylation of signaling molecules culminates in higher kinase activity of these pathways in the clones. Next, alterations in the DBA of NF-κB and AP-1 in the clones were examined. Biotin-labeled oligonucleotides probes comprising the NF-κB (Harada, H. et al., Mol. Cell Biol., 14:1500-9 (1994)) and AP-1 (Lee, W et al., Cell, 49:741-52 (1987)) consensus binding sites were used in EMSAs which reveal that compared to wt cells NF-κB and AP-1 DBAs are increased in the clones and rituximab fails to reduce their DBA. Rituximab-induced decrease in NF-κB and AP-1 DBA was only observed in wt cells. Specificity of EMSA was corroborated using appropriate controls (FIG. 8C). Specific inhibitors (DHMEQ, and PD098059) preferentially reduced NF-κB and AP-1 DBA. Collectively, these results show that NF-κB and ERK1/2 pathways are constitutively hyper-activated in RR clones and denote the inability of rituximab to negatively regulate the activities of these pathways in the clones unlike wt cells. Hyper-activation of these pathways will lead to enhanced transcription of their respective anti-apoptotic target genes leading to higher drug-resistance of the clones.

### Example 9E

### Chemo-sensitization of RR Clones by Pharmacological Inhibitors of ERK1/2 and NF-κB Pathways

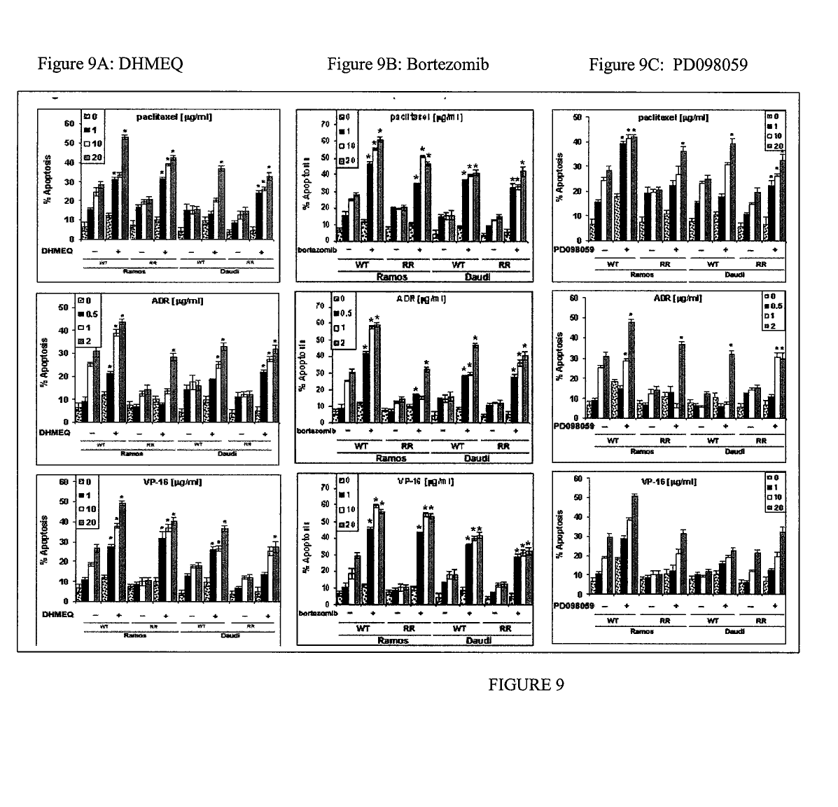

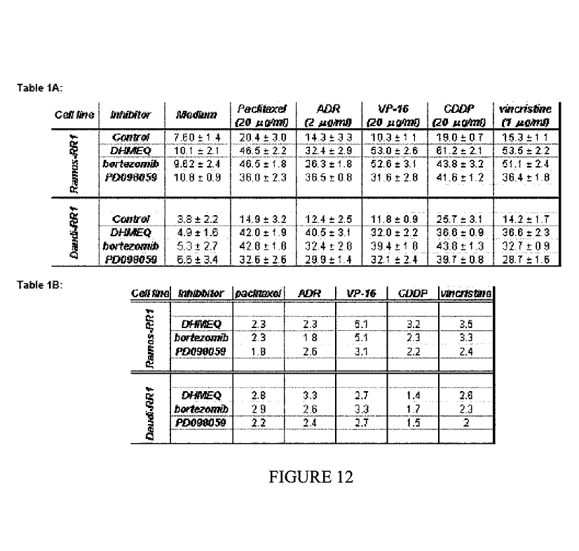

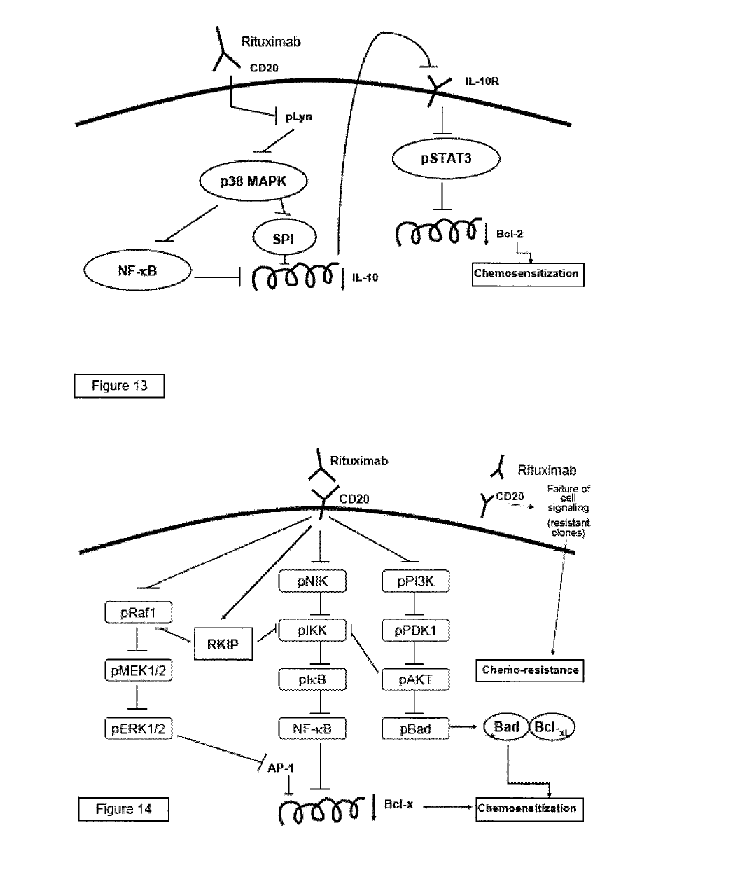

The NF-κB and ERK1/2 pathways are hyper-activated in RR clones leading to over-expression of Bcl-2, Bcl-_{XL}, Mcl-1 all of which are unaffected by rituximab, prompting us to investigate whether inhibition of these pathways or Bcl-2 members can reverse chemo-resistance. Since these pathways have higher activities in RR clones, higher concentrations of inhibitors were required for chemo-sensitization of the clones than those used for wt cells; non-toxic effective concentrations of which were determined by pilot studies (data not shown). Cells were left either untreated or pre-treated with DHMEQ (36), bortezomib (Goy, A. et al., Clin. Lymphoma, 4:230-7 (2004)) and PD098059 (Alessi, D. et al., J Biol. Chem., 270:27489-94 (1995)). Escalating concentrations of various drugs were then added and the percentage of apoptosis was measured. In Ramos-RR1, while DHMEQ induces 10.1±2.1% apoptosis, it significantly augments the apoptotic effects of drugs (paclitaxel: 20.4±3.0%→46.5±2.3%, ADR: 14.3±3.3%→32.4±2.9%, VP-16: 10.3±1.1%→53.0±2.6%, CDDP: 19.0±0.7%→61.2±2.1%, vincristine: 15.3±1.1%→53.5±2.2%). In Daudi-RR1 DHMEQ induces 4.9±1.6% apoptosis and augments drug efficacy (paclitaxel: 14.9±3.2%→42.0±1.9%, ADR: 12.4±2.5%→40.5±3.1%, VP-16: 11.8±0.9%→32.0±2.2%, CDDP: 25.7±3.1%→36.6±0.9%, vincristine: 14.2±1.7%→36.6±2.3%) (FIG. 9A-C). Similar patterns of significant dose-dependent chemo-sensitization of RR clones were observed, though for simplicity only the values pertaining to the highest drug concentration are presented (FIG. 12, Table 1A). Enhanced cytotoxicity by DHMEQ was 2.3-5.1 and 1.4-3.3 folds, by bortezomib was 1.8-5.1 and 1.7-3.3 folds and by PD098059 was 1.8-3.1 and 1.5-2.7 folds in Ramos-RR1 and Daudi-RR1, respectively (FIG. 12, Table 1B). The ability of inhibitors to significantly chemo-sensitize the RR clones (1.5-5.1 folds), indicates that inhibition of NF-κB and ERK1/2 pathways can avert chemo-/rituximab-resistance in clones to sub-toxic drug concentrations.

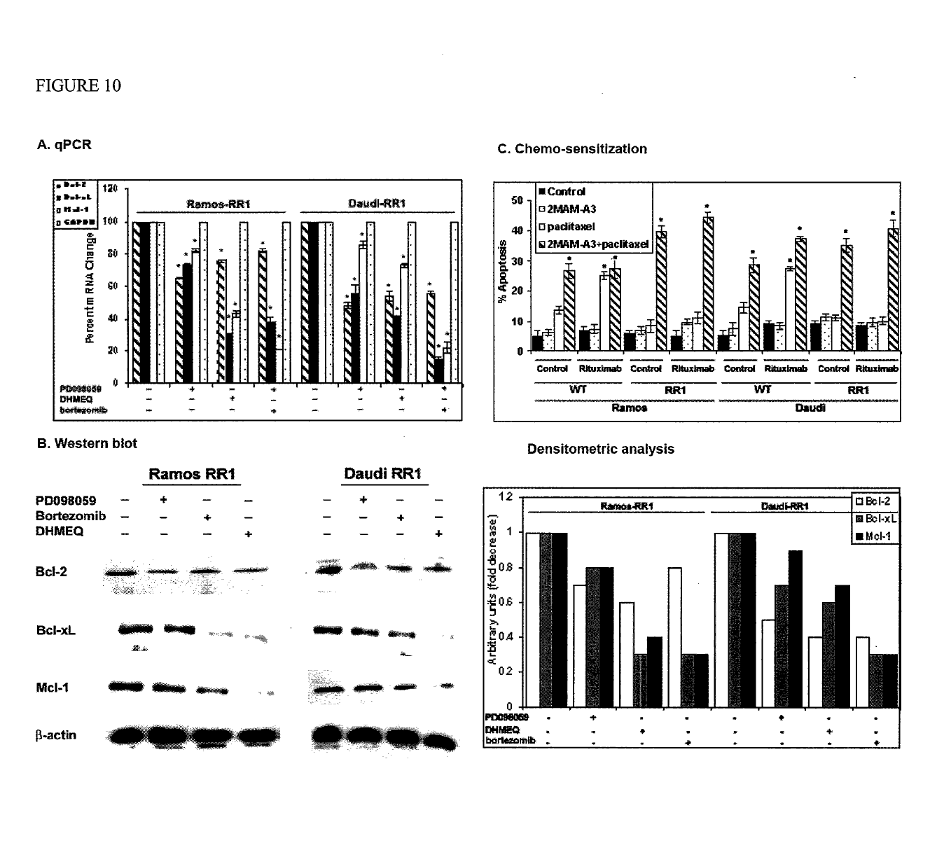

Since inhibitors efficiently chemo-sensitized the clones, we assessed their effect on expression of the resistant-factors. As depicted by qPCR inhibitors reduced mRNA levels of Bcl-2, Bcl-_{XL} and Mcl-1 by 1.2-4.8 folds and 1.2-6.6 folds (FIG. 10A), and immunoblot showed 1.25-3.3 folds and 1.1-3.3 folds decrease in their protein levels in Ramos-RR1 and Daudi-RR1, respectively (FIG. 10B) further showing the involvement of NF-κB and ERK1/2 pathways in the expression of resistant-factors.

The chemo-protective role of the over-expressed Bcl-2, Bcl-_{XL} and Mcl-1 in clones was further confirmed by pre-treatment with 2MAM-A3 (Tzung, S-P et al., Nature Cell Biology, 3:183-91 (2001)) which chemo-sensitized them at levels comparable with those achieved by rituximab. In wt Ramos 2MAM-A3 augmented paclitaxel cytotoxicity by 1.96 folds (13.6±1.3%→26.7±2.2%), which was 4.71 folds (8.42±2.1%→39.7±2.4%) in Ramos-RR1. Similar pattern was observed in Daudi (wt: 1.98 fold, RR1: 3.2 folds) (FIG. 10C). These findings support our contention that over-expression of anti-apoptotic Bcl-2 members upon prolonged rituximab treatment protects the cells against drug-induced apoptosis and their functional impairment is critical for chemo-sensitization.

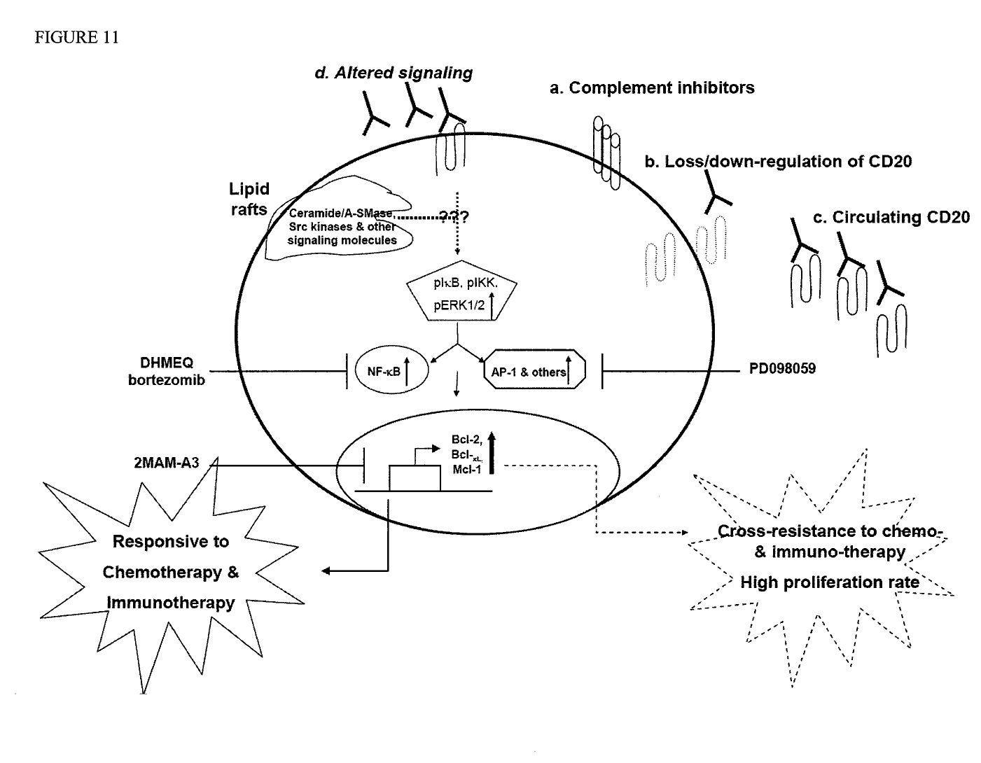

These B-NHL RR clones which exhibit a different phenotypic profile compared to wt cells. Using various biochemical and functional assays, compared to wt cells, RR clones express lower levels of surface CD20, do not respond to either growth inhibition by rituximab, rituximab-mediated CDC or cross-linked rituximab-induced apoptosis. Further, RR clones are not chemo-sensitized by rituximab and exhibit higher drug-resistance (1.41-2.97 fold). Two major survival pathways (NF-κB and ERK1/2) are constitutively hyper-activated in the clones leading to over-expression of resistant-factors (Bcl-2, Bcl-_{XL} , Mcl-1) (˜2.3-5.0 fold). Pharmacological inhibition of Bcl-2 family members (by 2MAM-A3), NF-KB (by DHMEQ, bortezomib), and ERK1/2 pathways (by PD098059) averts the drug-resistant phenotype and the clones undergo apoptosis in response to low concentrations of various drugs (FIG. 11).

Reducing the proliferation rate of tumor cells is postulated as one of rituximab's potential modes of action (Maloney, D., J Clin Oncol., 23:6421-8 (2005); Smith, M., Oncogene, 22:7359-68 (2003)). Rituximab treatment of wt cells reduces their growth rate (Jazirehi, A. et al., Mol. Cancer Therapeutics, 2:1183-93 (2003)), though, the clones grew at similar rates as wt cells and rituximab was incapable of inhibiting their growth (FIG. 6B) consistent with the higher growth rate and progressive nature of relapsed lymphomas, suggesting that RR clones have lost the ability to undergo rituximab-mediated growth-reduction possibly through a defective ceramide (CER)- acid sphingomyelinase (A-SMase) pathway (Bezombes, C. et al., Blood, 104:1166-73 (2004)). Compared to wt cells, RR clones exhibit higher resistance to CDC. Rituximab pretreatment significantly enhanced CDC in wt cells; an effect that was not noticed in the clones. Increasing serum concentrations enhanced rituximab-mediated CDC in wt cells but not in clones (FIG. 6D) consistent with our preliminary findings showing increased expression of complement inhibitors on the clones (data not shown). Further studies are warranted to delineate the role of complement inhibitors in CDC-resistance of the RR clones. Cross-linked rituximab induced significant apoptosis (FIG. 6E) and cytostasis (data not shown) on wt cells. Neither induction of apoptosis nor growth inhibition was observed on treatment of the clones with cross-linked rituximab, suggesting that RR clones have developed higher threshold in response to biological effects of cross-linked rituximab. The above effects were independent of rituximab concentration as higher (2.5-5 fold) concentrations of rituximab failed to reverse the phenotype of the clones (data not shown).

Rituximab binds to B-cell restricted cell surface CD20, thus, exerting its effects. Various mechanisms are postulated for rituximab-resistance including transient CD20 down-regulation (Kennedy, A. et al., J Immunol., 172:3280-8 (2004)), loss of CD20 (Haidar, J. et al., Eur J Haematol, 70:330-2 (2003)) and circulating CD20 (Manshouri, T. et al., Blood, 101:2507-13 (2003)). Substantial CD20 surface expression was detected on wt cells, still, RR clones exhibited about 50% reduction in surface CD20 expression (FIG. 6A). The significance of reduced CD20 expression in clones requires further investigation as several lines of evidence suggest that the biological effects of rituximab therapy is autonomous of the intensity of surface CD20 even in vivo (Kennedy, A. et al., J immunol., 172:3280-8 (2004)). Thus, failure of rituximab to exert its biological effects on RR clones may be independent of diminished CD20. It may be due to the activation status of the clones and/or aberrant cellular signaling as deregulation of the signaling pathways or aberrant expression of signaling molecules contribute to acquired chemo-resistance (Manshouri, T. et al., Blood, 101:2507-13 (2003); Pommier, Y. et al., Oncogene, 23:2934-49 (2004)). Analysis of the signaling pathways in the clones revealed hyper-activation of ERK1/2 and NF-κB pathways leading to over-expression of their down-stream resistant- factors Bcl-2, Bcl-_{XL} and Mcl-1, and the exhibition of higher drug-resistance (1.41-2.97 fold) concordant with the protective role of Bcl-2 family members (Wada, T. et al., Oncogene, 23:2838-49 (2004); Minn, A. et al., Blood, 86:1903-10 (1995)) suggesting that the selective pressure applied by prolonged rituximab treatment has co-selected for cells that express higher levels of anti-apoptotic proteins, which have lost the capacity to undergo apoptosis in response to various stimuli. The possibility of the pre-existence of resistant cells in the native culture is not ruled out. In fact, by no criteria thus far rituximab has altered the biological properties of 100% of the cells. However, the resistant sub-clones in native culture will dominate the sensitive population on long-term rituximab treatment as the sensitive cells will be eliminated over time. However, there is no unequivocal evidence, as yet, that this is the dominant mechanism in vivo. Since drugs utilize apoptosis as a mean of exerting their effects, thus, drug-resistant tumors develop cross-resistance to apoptosis induced by structurally and functionally distinct stimuli including immunotherapy and vs. Thus, as NHL cells develop resistance to rituximab, they may also develop cross-resistance to drugs and immune system, consistent with our observation that drug-resistant RR clones also exhibit higher resistance to tumor necrosis factor (TNF)-related apoptosis-inducing ligand (TRAIL) and anti-Fas agonistic Ab (Jazirehi, A. et al., Blood, 106:1514 (2005)). Rituximab pre-treatment of wt cells, not the clones, efficiently sensitized them to drugs and TRAIL (Jazirehi, A. et al., Blood, 106:1514 (2005)).

Unlike wt cells (Jazirehi, A. et al., Cancer Research, 64:7117-26 (2004); Jazirehi, A. et al., Cancer Research, 65:264-76 (2005)), rituximab was incapable of inhibiting hyper-activated NF-κB and ERK1/2 pathways in the clones. Thus, constitutive hyper-activation of these pathways appear to confer higher drug-resistance (Ghosh, S. et al., Cell, 109:S81-S96 (2002); Dent, P. et al., Clin Cancer Res, 7:775-83 (2001)), and their inhibition could potentially avert the chemo-resistance; prompting us to evaluate the chemo-sensitizing effects of specific inhibitors of NF-κB and ERK1/2 pathways as well as bortezomib (Velcade) (Goy, A. et al., Clin. Lymphoma, 4:230-7 (2004)). DHMEQ is a unique inhibitor of NF-κB acting at the level of nuclear translocation, completely inhibits NF-κB DBA, inhibits the growth of human hormone-refractory prostate and bladder cancer cells and at high concentrations induces apoptosis (Ariga, A. et al., J. Biol. Chem., 277:24626-30 (2002); Kikuchi, E. et al., Cancer Res., 63:107-10 (2003)). PD098059 exerts its effects by specifically binding to inactive form of MEK1/2 and prevents its activation by Raf-1, thus, inhibiting ERK1/2 activation (Alessi, D. et al., J. Biol. Chem., 270:27489-94 (1995)). Bortezomib is approved for the treatment of multiple myeloma and has significant single-agent activity against certain subtypes of NHL (O'Connor, O. et al., J Clin Oncol., 23:676-84 (2005)). These inhibitors efficiently sensitized the RR clones to structurally and functionally distinct drugs including topoisomerase II inhibitor, DNA alkylating agents, and microtubule poisons, albeit to varying degrees (FIG. 12, Table 1). The inhibitors also reduced Bcl-2, Bcl-_{XL}, MCl-1 levels further suggesting that deregulated signaling culminates in over-expression of anti-apoptotic proteins in RR clones leading to higher drug-resistance. The chemo-protective role of Bcl-2 family members was confirmed by using 2MAM-A3, a specific inhibitor that binds to the hydrophobic groove formed by the highly conserved BH1, BH2 and BH3 domains, thus, impairing the function of Bcl-2, Bcl-_{XL} and Mcl-1 (Tzung, S-P et al., Nature Cell Biology, 3:183-91 (2001)). 2MAM-A3 efficiently chemo-sensitized the clones, further attesting that higher expression of resistant-factors protects RR clones from drug-induced apoptosis. Hence, aberrations in the normal dynamics of cellular survival pathways upon continuous rituximab exposure contribute to acquired rituximab- and/or drug-resistance while interruption of these pathways leads to chemo-sensitization of RR clones. Thus, functional impairment of anti-apoptotic proteins is critical for reversion of chemo-resistance.

The nature of the molecular cues that trigger the aberrant activation of survival pathways in the clones, hence their altered phenotype, are unclear at present. In wt Daudi rituximab induces a rapid and transient increase in A-SMase activity parallel with cellular CER generation in lipid rafts. These cells externalize both CER and A-SMase which co-localize with CD20. Also, rituximab-induced growth inhibition may be mediated through a CER-dependent pathway (Bezombes, C. et al., Blood, 104:1166-73 (2004)). Preliminary observations suggest rituximab-induced A-SMase translocation and CER generation at cell surface is reduced in clones (Jazirehi, A. et al., Proceedings of AACR, 47 (2006)). Since micro-domains serve as signaling platforms, these data suggest that rituximab-resistance in clones is, partly, due to faulty mobilization of the signalling molecules to lipid rafts and a crippled CER/A-SMase pathway.

The present findings are the first report on the establishment of an in vitro model of RR NHL clones which shows repeated rituximab exposure results in loss of rituximab's ability to regulate molecular switches leading to constitutive hyper-activation of survival pathways, over-expression of resistant factors and increased apoptosis threshold. Accordingly, rituximab fails to exert anti-lymphoma effects and RR clones develop higher drug-resistance, which can account for the treatment-refractory and the aggressive nature of clinical rituximab-/drug-resistant NHL. FIG. 6 illustrates potential mechanisms of RR. The observed drug- and rituximab-resistance of RR clones mimic those observed previously (Olejniczak, S. et al., Blood, 106:4819 (2005)). Though, RR clones are still amenable to chemotherapy using specific molecular targeting of the components of deregulated pathways. Our studies identify several such targets for potential molecular intervention in the treatment of rituximab-/drug-resistant NHL. Analysis of dynamics of survival pathways in patient-derived specimens may also serve as biomarkers in choosing treatment options. Such patients may be suitable candidates for alternative (e.g., targeted therapy) over conventional regimens.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of diagnosing a cancer or providing a prognosis for patient having a cancer that has altered expression of molecular signaling pathways triggered by rituximab, the method comprising the steps of:
(a) contacting a tissue sample of the cancer with an antibody that specifically binds to protein that is part of a molecular signaling pathway triggered by rituximab; and
(b) determining whether or not expression of the protein is altered in the sample, thereby diagnosing or providing the prognosis for the cancer.

2. The method of claim 1, wherein the cancer is a CD20 expressing cancer.

3. The method of claim 1, wherein the cancer is lymphoma.

4. The method of claim 1, wherein the molecular signaling pathway is functional or activated AKT or NFκB.

5. The method of claim 1, wherein the protein is PTEN, AKT, Fas, YY1, NFκB, NIK, IKK, or IKB.

6. The method of claim 1, wherein the protein is Bcl-2, Bcl-_{XL}, AP-1 or STAT3.

7. The method of claim 1, wherein the pathway is selected from a p38 MapK/ Stat 3, Raf 1/MEK 1/2/ ERK 1/2, NfκB or Akt pathway.

8. The method of claim 4, wherein the tissue sample is at least one of fixed or embedded in paraffin.

9. The method of claim 1, wherein the antibody is a monoclonal antibody.

10. The method of claim 1, wherein the tissue sample is a metastatic cancer tissue sample.

11. The method of claim 1, wherein the tissue sample is from blood, bone marrow, prostate, ovary, bone, lymph node, liver, kidney, or sites of metastases.

12. A method of diagnosing a cancer or providing a prognosis for a patient having a cancer that that has altered expression of molecular signaling pathways triggered by rituximab, the method comprising the steps of:
(a) contacting a tissue sample of the cancer with a primer set of a first oligonucleotide and a second oligonucleotide that each specifically hybridize to a nucleic acid encoding a protein that is part of a molecular signaling pathway triggered by rituximab; and
(b) determining whether or not expression of the nucleic acid is altered in the sample; thereby diagnosing the cancer.

13. The method of claim 12, wherein amplifying YY1 nucleic acid is amplified in the sample; and the expression of YY1 nucleic acid is determined, wherein it is determined whether or not the cancer overexpresses YY1.

14. A method of localizing a cancer that in vivo, the cancer having altered expression of molecular signaling pathways triggered by rituximab, the method comprising the step of imaging in a subject a cell a polypeptide member of the molecular signaling pathways triggered by rituximab, thereby localizing the cancer in vivo.

15. A method of identifying a compound that inhibits a cancer that has an altered molecular signaling pathways triggered by rituximab, the method comprising the steps of:
(a) contacting a cell expressing a polypeptide member of the molecular signaling pathways triggered by rituximab with a compound; and
(b) determining the effect of the compound on the polypeptide; thereby identifying a compound that inhibits the cancer.

16. A method of identifying a compound that inhibits a therapy resistant cancer, the method comprising the steps of:
(a) contacting a cell expressing a polypeptide member of a molecular signaling pathways triggered by rituximab with a compound; and
(b) determining the effect of the compound on the polypeptide; thereby identifying a compound that inhibits the therapy resistant cancer.

17. A method of treating or inhibiting a cancer in a subject that that has an altered molecular signaling pathway triggered by rituximab comprising administering to the subject a therapeutically effective amount of one or more inhibitors that modulates a polypeptide member of a molecular signaling pathway triggered by rituximab.

18. The method of claim 17, wherein the inhibitor is a small organic molecule or a chemical inhibitor.

19. The method of claim 17, wherein the inhibitor is an NO donor.

20. The method of claim 18, wherein the NO donor is selected from the group consisting of L-arginine, amyl nitrite, isoamyl nitrite, nitroglycerin, isosorbide dinitrate, isosorbide-2-mononitrate, isosorbide-5-mononitrate, erythrityl tetranitrate, pentaerythritol tetranitrate, sodium nitroprusside, 3-morpholinosydnonimine, molsidomine, N-hydroxyl-L-arginine, S,S-dinitrosodthiol, ethylene glycol dinitrate, isopropyl nitrate, glyceryl-1-mononitrate, glyceryl-1,2-dinitrate, glyceryl-1,3-dinitrate, glyceryl trinitrate, butane-1,2,4-triol trinitrate, N,O-diacetyl-N-hydroxy-4-chlorobenzenesulfonamide, N^{G}-hydroxy-L-arginine, hydroxyguanidine sulfate, (±)-S-nitroso-N-acetylpenicillamine, S-nitrosoglutathione, (±)-(E)-ethyl-2-[(E)-hydroxyimino]-5-nitro-3-hexeneamide (FK409), (±)-N-[(E)-4-ethyl-3-[(Z)-hydroxyimino]-5-nitro-3-hexen-1-yl]-3-pyridinecarboxamide (FR144420), 4-hydroxymethyl-3-furoxancarboxamide, (Z)-1-[2-(2-Aminoethyl)-N-(2-ammonioethyl)amino]diazen-1-ium-1,2-diolate; NOC-18; 3,3-bis(aminoethyl)-1-hydroxy-2-oxo-* 1-triazene (DETA/NONOate), NO gas, and mixtures thereof.

21. The method of claim 17, wherein the inhibitor is an siRNA.

22. The method of claim 17, wherein the inhibitor is an antimitotic drug.

23. The method of claim 22, wherein the antimitotic drug is selected from the group consisting of vinca alkaloids and taxanes.

24. A method of treating or inhibiting a therapy resistant cancer in a subject comprising administering to the subject a therapeutically effective amount of one or more inhibitors of a polypeptide member of a molecular signaling pathway triggered by rituximab.

25. The method of claim 24, wherein the inhibitor is a small organic molecule or a chemical inhibitor.

26. The method of claim 24, wherein the therapy resistant cancer has altered expression of molecular signaling pathways triggered by rituximab, and the therapy-resistant cancer was diagnosed by:
(a) contacting a tissue sample of the cancer with an antibody that specifically binds to protein that is part of a molecular signaling pathway triggered by rituximab; and
(b) determining whether or not expression of the protein is altered in the sample, thereby diagnosing or providing the prognosis for the cancer.

27. The method of claim 24, wherein the one or more inhibitors are administered concurrently with another cancer therapy.

28. A method of treating a patient having a CD-20 expressing cancer, said method comprising administering to the patient a modulator of a p38 MapK/ Stat 3, Raf 1/MEK 1/2/ERK 1/2, Nf-kappa B or Akt pathway.

29. The method of claim 28, wherein the CD-20 expressing cancer is selected from the group consisting of lymphoma, B-acute lymphoblastic lymphoma, non-Hodgkin's lymphoma, Burkitt's small cell, and large cell lymphomas, chronic lymphocytic leukemia, Hodgkin's lymphoma, leukemia, acute myelogenous leukemia, acute lymphoblastic leukemia, chronic modulator myelogenous leukemia, and multiple myeloma.

30. The method of claim 28, wherein said modulator is a pro-apoptosis modulator of a p38 MapK/ Stat 3, Raf 1/MEK 1/2/ ERK 1/2, Nf-kappa B or Akt pathway.

31. The method of claim 28, wherein said modulator binds PTEN, AKT, Fas, YY1, NFκB, NIK, IKK, Bcl-2, Bcl-_{XL}, AP-1, STAT3 or IKB.

32. The method of claim 28, wherein the CD-20 expressing cancer was identified to have a hyperactive p38 MapK/ Stat 3, Raf 1/MEK 1/2/ ERK 1/2, Nf-kappa B or Akt pathway.

33. The method of claim 32, wherein the CD-20 expressing cancer was identified to have a hyperactive p38 MapK/ Stat 3, Raf 1/MEK 1/2/ ERK 1/2, Nf-kappa B or Akt pathway by determining whether or not expression or amounts of a protein of the pathway is altered.

34. The method of claim 33, wherein the protein selected from the group consisting of PTEN, AKT, Fas, YY1, NFκB, NIK, IKK, Bcl-2, Bcl-_{XL}, AP-1, STAT3, and IKB.
